SEQUENCE LISTING (73 sequences)

SEQ ID NO: 1 - DNA, 513 bp, Brassica napus
atggtgagaaaatgcagaaaaactaaagggacggtgggagcttcgtctacgtatatgcagcttcgcagccggagaatcgtttacagatcggaaaaagctagctcgtcgtcgtcgtcttgttgcgcgagtaacaacaatggagttatagatcttgaggaggaaagagatggtgagactgaaacgtcgtcgtgtcgacggagtagtaagaggaagctatttgaaaaccttagagaaaaagaatctatggagaattcacagcaaatcgtagctggttttgattccgccgtgaaagaatcatcggattgttgttgcagccggagaacatctttgtcaacgacggaggagaaggggaaatcagcgacggagcaaccaccaacggcagtggagattgaagattttttcgtggaagctgagaaacagctccatgataatttcaagaagaagtataactttgatttcgaaaaggagaagccattagaaggacgctacgagtgggttaaattatcagagtaa

SEQ ID NO: 2 - protein, 170 aa, Brassica napus
MVRKCRKTKGTVGASSTYMQLRSRRIVYRSEKASSSSSSCCASNNNGVIDLEEERDGETETSSCRRSSKRKLFENLREKESMENSQQIVAGFDSAVKESSDCCCSRRTSLSTTEEKGKSATEQPPTAVEIEDFFVEAEKQLHDNFKKKYNFDFEKEKPLEGRYEWVKLSE

SEQ ID NO: 3 - protein, 170 aa, Artificial Sequence
  Brassica napus
MVRKCRKTKGTVGASSTYMQLRSRRIVYRSEKASSSSSSCCASNNNGVIDLEEERDGETETSSCRRSSKRKLFENLREKESMENSQQIVAGFDSAVKESSDCCCSRRTSLSTTEEKGKSATEQPPTAVEIEDFFVEAEKQLHDNFKKKYNADAEKEKPLEGRYEWVKLSE

SEQ ID NO: 4 - protein, 170 aa, Artificial Sequence
  Brassica napus
MVRKCRKTKGTVGASSTYMQLRSRRIVYRSEKASSSSSSCCASNNNGVIDLEEERDGETETSSCRRSSKRKLFENLREKESMENSQQIVAGFDSAVKESSDCCCSRRTSLSTTEEKGKSATEQPPTAVEIEDFFVEAEKQLHDNFKKKANADAEKEKPLEGRYEWVKLSE

SEQ ID NO: 5 - protein, 191 aa, Arabidopsis thaliana
MVRKYRKAKGIVEAGVSSTYMQLRSRRIVYVRSEKSSSVSVVGDNGVSSSCSGSNEYKKKELIHLEEEDKDGDTETSTYRRGTKRKLFENLREEEKEELSKSMENYSSEFESAVKESLDCCCSGRKTMEETVTAEEEEKAKLMTEMPTESEIEDFFVEAEKQLKEKFKKKYNFDFEKEKPLEGRYEWVKLE

SEQ ID NO: 6 - DNA, 579 bp, Zea mays
atgggcaagtacatgcgcaaggccaaggcttccagcgaggttgtcatcatggatgtcgccgccgctccgctcggagtccgcacccgagcgcgcgccctcgcgctgcagcgtctgcaggagcagcagacgcagtgggaggaaggtgctggcggcgagtacctggagctaaggaaccggaggctggagaagctgccgccgccgccggcgaccacgaggaggtcgggcgggaggaaagcggcagccgaggccgccgcaactaaggaggctgaggcgtcgtacggggagaacatgctagagttggaggccatggagaggattaccagggagacgacgccttgcagcttgattaacacccagatgactagcactcctgggtccacgagatccagccactcttgccaccgcagggtgaacgctcctccggtgcacgccgtcccaagttcgagggagatgaatgagtacttcgctgccgaacagcgacgccaacaacaggatttcattgacaagtacaacttcgatcctgcaaacgactgccctctcccaggcaggtttgagtgggtgaagctagactgactcgag

SEQ ID NO: 7 - protein, 190 aa, Zea mays
MGKYMRKAKASSEVVIMDVAAAPLGVRTRARALALQRLQEQQTQWEEGAGGEYLELRNRRLEKLPPPPATTRRSGGRKAAAEAAATKEAEASYGENMLELEAMERITRETTPCSLINTQMTSTPGSTRSSHSCHRRVNAPPVHAVPSSREMNEYFAAEQRRQQQDFIDKYNFDPANDCPLPGRFEWVKLD

SEQ ID NO: 8 - protein, 190 aa, Artificial Sequence; identical to SEQ ID NO: 7
  Zea mays

SEQ ID NO: 9 - protein, 190 aa, Artificial Sequence
  Zea mays
MGKYMRKAKASSEVVIMDVAAAPLGVRTRARALALQRLQEQQTQWEEGAGGEYLELRNRRLEKLPPPPATTRRSGGRKAAAEAAATKEAEASYGENMLELEAMERITRETTPCSLINTQMTSTPGSTRSSHSCHRRVNAPPVHAVPSSREMNEYFAAEQRRQQQDFIDKANADAANDCPLPGRFEWVKLD

SEQ ID NO: 10 - DNA, 777 bp, Zea mays
atggggaagtacatgcgcaagtgcaggggcgccgcaggcgcggaggtcgccgccgtcgaggttacgcaggtcgtcggcgtccggacgaggtccaggtccgcggcggcgaccggcggtgtcgcgaaggtcgccccgaggaggaagagggcgccggcgggggagcctgctgccgccgtgagcgctggtggggacggcggaagctgctacatccacctgcgtagccgcatgctgttcatggcaccgcctcagccgcagccgtcggttgactcggttccgaccccggtggaggctgctgatggcgctgcaggacagcagggcgcggcgctcgcggccgggctctcgcgttgctccagcacggcgtcgtcggtgaacttgggcttggggggtcagcgcgggagccacacctgccgctcctacgacgctgcagaggctggcggggatcacgtcctggtggatgtctcggcggcgagcaactccgggagcggcccagaccgcgagaggcgagagacgacgccatcgagccgggcgcacggcgagctgagcgatctggagtcggatctggcggggcacaagactggcccgtcgctaccggcggcaacgccggctgcggagctgatcgtgccgccagcacacgagatccaggagttcttcgccgccgccgaggcggcccaggccaagcgctttgcttccaagtacaacttcgacttcgtccgcggcgtgcccctcgacgccggcggccggttcgagtgggcgccggtggtcagcatctgactcgag

SEQ ID NO: 11 - protein, 256 aa, Zea mays
MGKYMRKCRGAAGAEVAAVEVTQVVGVRTRSRSAAATGGVAKVAPRRKRAPAGEPAAAVSAGGDGGSCYIHLRSRMLFMAPPQPQPSVDSVPTPVEAADGAAGQQGAALAAGLSRCSSTASSVNLGLGGQRGSHTCRSYDAAEAGGDHVLVDVSAASNSGSGPDRERRETTPSSRAHGELSDLESDLAGHKTGPSLPAATPAAELIVPPAHEIQEFFAAAEAAQAKRFASKYNFDFVRGVPLDAGGRFEWAPVVSI

SEQ ID NO: 12 - protein, 256 aa, Artificial Sequence
  Zea mays
MGKYMRKCRGAAGAEVAAVEVTQVVGVRTRSRSAAATGGVAKVAPRRKRAPAGEPAAAVSAGGDGGSCYIHLRSRMLFMAPPQPQPSVDSVPTPVEAADGAAGQQGAALAAGLSRCSSTASSVNLGLGGQRGSHTCRSYDAAEAGGDHVLVDVSAASNSGSGPDRERRETTPSSRAHGELSDLESDLAGHKTGPSLPAATPAAELIVPPAHEIQEFFAAAEAAQAKRFASKYNADAVRGVPLDAGGRFEWAPVVSI

SEQ ID NO: 13 - protein, 256 aa, Artificial Sequence
  Zea mays
MGKYMRKCRGAAGAEVAAVEVTQVVGVRTRSRSAAATGGVAKVAPRRKRAPAGEPAAAVSAGGDGGSCYIHLRSRMLFMAPPQPQPSVDSVPTPVEAADGAAGQQGAALAAGLSRCSSTASSVNLGLGGQRGSHTCRSYDAAEAGGDHVLVDVSAASNSGSGPDRERRETTPSSRAHGELSDLESDLAGHKTGPSLPAATPAAELIVPPAHEIQEFFAAAEAAQAKRFASKANADAVRGVPLDAGGRFEWAPVVSI

SEQ ID NO: 14 - DNA, 648 bp, Zea mays
atggggaagtacatgcgcaagggcaaggtgtccggggaggtcgccgtcatggaggtacccggcggcgcgctgctcggcgtccgcacccgctcccgcacgctcgcgctgcagcgcgcgcagaggccgctcgacaagggcgacgcggaggacgccgccgcggagtacctcgaactcaggagccggaggctggagaagccgcacaaggagcatccgtcgccgcccgcgaccgcgaccaagaggggcgccgggaggaaggccgccgccgccgccgcggtgcagcacgtgctgatgcaggacgaggtcgaggtcgaggtctcgttcggggacaacgtgcttgacttggacaccatggaaaggagtaccagagagacaacaccgtgcagcctgattaggaacccagagatgataagcaccccagggtccacaactaaaagcaaaaccagcagcaactcgacgacttcccgccgcagaacggaggaaaccccgagctgccggttcataccgagttcgctcgaaatggaggagttcttctcggcggccgagcaacaggagcagcatagcttcagggagaagtacaacttctgtcccgtgaacgactgtcctctccctggccggtacgaatgggcgaggctagactgctagctcgag

SEQ ID NO: 15 - protein, 213 aa, Zea mays
MGKYMRKGKVSGEVAVMEVPGGALLGVRTRSRTLALQRAQRPLDKGDAEDAAAEYLELRSRRLEKPHKEHPSPPATATKRGAGRKAAAAAAVQHVLMQDEVEVEVSFGDNVLDLDTMERSTRETTPCSLIRNPEMISTPGSTTKSKTSSNSTTSRRRTEETPSCRFIPSSLEMEEFFSAAEQQEQHSFREKYNFCPVNDCPLPGRYEWARLDC

SEQ ID NO: 16 - protein, 213 aa, Artificial Sequence
  Zea mays
MGKYMRKGKVSGEVAVMEVPGGALLGVRTRSRTLALQRAQRPLDKGDAEDAAAEYLELRSRRLEKPHKEHPSPPATATKRGAGRKAAAAAAVQHVLMQDEVEVEVSFGDNVLDLDTMERSTRETTPCSLIRNPEMISTPGSTTKSKTSSNSTTSRRRTEETPSCRFIPSSLEMEEFFSAAEQQEQHSFREKYNACAVNDCPLPGRYEWARLDC

SEQ ID NO: 17 - protein, 213 aa, Artificial Sequence
  Zea mays
MGKYMRKGKVSGEVAVMEVPGGALLGVRTRSRTLALQRAQRPLDKGDAEDAAAEYLELRSRRLEKPHKEHPSPPATATKRGAGRKAAAAAAVQHVLMQDEVEVEVSFGDNVLDLDTMERSTRETTPCSLIRNPEMISTPGSTTKSKTSSNSTTSRRRTEETPSCRFIPSSLEMEEFFSAAEQQEQHSFREKANACAVNDCPLPGRYEWARLDC

SEQ ID NO: 18 - DNA, 45 bp, Artificial Sequence
  primer
cattgacaagtacaacgccgatgccgcaaacgactgccctctccc

SEQ ID NO: 19 - DNA, 45 bp, Artificial Sequence
  primer
gggagagggcagtcgtttgcggcatcggcgttgtacttgtcaatg

SEQ ID NO: 20 - DNA, 39 bp, Artificial Sequence
  primer
gcttccaagtacaacgccgacgccgtccgcggcgtgccc

SEQ ID NO: 21 - DNA, 39 bp, Artificial Sequence
  primer
gggcacgccgcggacggcgtcggcgttgtacttggaagc

SEQ ID NO: 22 - DNA, 43 bp, Artificial Sequence
  Zea mays
cagggagaagtacaacgcctctgccgtgaacgactgtcctctc

SEQ ID NO: 23 - DNA, 43 bp, Artificial Sequence
  primer
gagaggacagtcgttcacggcagaggcgttgtacttctccctg

SEQ ID NO: 24 - DNA, 45 bp, Artificial Sequence
  primer
ggatttcattgacaaggccaacgccgatgccgcaaacgactgccc

SEQ ID NO: 25 - DNA, 45 bp, Artificial Sequence
  primer
gggcagtcgtttgcggcatcggcgttggccttgtcaatgaaatcc

SEQ ID NO: 26 - DNA, 45 bp, Artificial Sequence
  primer
gcgctttgcttccaaggccaacgccgacgccgtccgcggcgtgcc

SEQ ID NO: 27 - DNA, 45 bp, Artificial Sequence
  primer
ggcacgccgcggacggcgtcggcgttggccttggaagcaaagcgc

SEQ ID NO: 28 - DNA, 45 bp, Artificial Sequence
  primer
gcttcagggagaaggccaacgcctctgccgtgaacgactgtcctc

SEQ ID NO: 29 - DNA, 45 bp, Artificial Sequence
  primer
gaggacagtcgttcacggcagaggcgttggccttctccctgaagc

SEQ ID NO: 30 - protein, 253 aa, Sorghum bicolor
MGKYMRSKCRGAAGEEVAAVEVTQVVGVRTRSRSAAATGGVAKVAPRRKKALTPAANVPAGEPAAVGAGGDGGSCYIHLRSRMLFMAPPQPQPSVPTPAEAAGAGQQGAALVAGLSRCSSTASSVDVGGQNKSHACRSGAAEVGGDHAPDVSASNSGNGPDRERREMTPSSRAHGELSDLESDLAGHKTGRSLPAATSAPVPIVPPAQEIEEFFAAAEAAQAKRFASKYNFDFVRGVPLEAGGRFEWTPVVSI

SEQ ID NO: 31 - protein, 262 aa, Oryza sativa
MGKYMRKFRGATGEELAAMEVTQVVGVRTRSRSAAAAGATTTKVKAASAASTRRRKALLPTAVVGTTRRDGGSCYLQLRSRMLFMAPPRPAPAARAPVVAEAAGSGNGAAAHAAAGLSRCSSTASSVDAAAQDRSLACRSDVAEAGSEHVPEGSASDSASGRDRERRETTPSSFLPGEVSDLESDLAGGQKRSRPLPSAATASAQQATRPKIPPAAEIEAFFAAAEEAEAKRFAAKYNFDVVRGVPLDAGRFEWTPVVSSRS

SEQ ID NO: 32 - DNA, 961 bp, Zea mays
gatccgattgactatctcattcctccaaacccaaacacctcaaatatatctgctatcgggattggcattcctgtatccctacgcccgtgtaccccctgtttagagaacctcccaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacccaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggaagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccttaa

SEQ ID NO: 33 - DNA, 992 bp, Hordeum vulgare
aattcaggagtcagcttttttttatttttcattacgaaaaaaatatgaaatcaaaaatggtttacatatttaaaggaaaaaatgttcatcgatttaaaaaaaattcatacctttcaaaaacgttcatcaattttttttaaaagttcactaaaaatgaaaaaaagttcatccaatttaaaaaaagttcattaattttttataaatttcaataaatttgaaaaaacttcataaaaattcaacaaaagttcattgaagtgaaaaaagctcataaatctttaaaaagtgcatccattttcaaaaagatgttcatcaaaattcaatatagttcaccaatattcaaaaaagttcattaatcttaaaaaatattcgctaaaatttaaaaaatgtttatcaatatttaaacggcgtctagatgagccggtctatttacaaacaccataggcgccaattaacaaaaatgcacgttagatcacgtctacggcgtcaaataggaaatgcccatcggccttactattaagagttgttttggttatcctttaggatttatgctgtgggctggacttaacacaaaacccacagccatggtaggccggaatctattattcagctcacaaacgatgttctactcaaaagaagaaaaaaatctgttgtcagaaaaggagaacaaaaaaggctcacgaacatgcggcggctcgcacaggtggccgtgagcttctgaatgacttggccacccggcatgtccactgcccccctagacggtgtgggtgggtggacaggtcaagcgcattgaacaaggtcaccctgcgttctgccacgaggccaactgcgtggccctcatgcaacgcgccttgctgccacttctacacacgccctcgccggccgaccgctgctataaaagcagctccccgttgcgtcctcgacggcatccatcgagagacgttcgcagcagcaaagagcacgcagcaactagctttcagttgtttcaacccctg

SEQ ID NO: 34 - DNA, 1224 bp, Zea mays
tactatagggcacgcgtggtcgacggcccgggctggtaaaaagtaattgaacccaaaatatcatggtatgtttggtgaagacagtgatcagtgatttttttatatctatatatatatcaaagatacttgattttctagaaggttctttttgttgttttcccttatgtttttacgcatgatgcaattctttttgagaggtttccgatgcattgatgttattgtattatctcctatatataggtcgacgtacattatgtattgcaataaccagttaactggatccagcttcgcttagtttttagtttttggcagaaaaaatgatcaatgtttcacaaaccaaatatttttataacttttgatgaaagaagatcaccacggtcatatctaggggtggtaacaaattgcgatctaaatgtttcttcataaaaaataaggcttcttaataaattttagttcaaaataaatacgaataaagtctgattctaatctgattcgatccttaaattttataatgcaaaatttagagctcattaccacctctagtcatatgtctagtctgaggtatatccaaaaagccctttctctaaattccacacccaactcagatgtttgcaaataaatactccgactccaaaatgtaggtgaagtgcaactttctccattttatatcaacatttgttattttttgtttaacatttcacactcaaaactaattaataaaatacgtggttgttgaacgtgcgcacatgtctcccttacattatgtttttttatttatgtattattgttgttttcctccgaacaacttgtcaacatatcatcattggtctttaatatttatgaatatggaagcctagttatttacacttggctacacactagttgtagttttgccacttgtctaacatgcaactctagtagttttgccacttgcctggcacgcgactctagtattgacacttgtatagcaaataatgccaatacgacacctggccttacatgaaacattatttttgacacttgtataccatgcaacattaccattgacatttgtccatacacattatatcaaatatattgagcgcatgtcacaaactcgatacaaagctggatgaccctccctcaccacatctataaaaacccgagcgctactgtaaatcactcacaacacaacacatatcttttagtaacctttcaataggcgtcccccaagaactagtaaac

SEQ ID NO: 35 - DNA, 1505 bp, Zea mays
ggtcgcggatcggggggttgtggtaggtgcgaagccgcacggttgccgttccctcacatcgcgccgggggatctggggcggtggcgggtgcggatgcggatctggagccgctcggtcatgactcccgagcatcacgccgaggccggggaggccaggggggttgcgacgccgatgacgggggcgggggaggcgctgaggacggggaggacgcgaggatgggcggcagcgacgttcgcggtaggggcgagtggggcggggatggtgaacttgacgggcggcatgtgggggcgcgcggcgccgccgcgtcagtctcctcctggcgggggcgcatggtagcagtcgtggcaactatggaggggggcaccgacgatgcgcggggatcgatgtggggcgccgacgacgaacggacgcggcagatgtggatcctcatcggatgcgccgatggaagggggtcgatgcagagcgtggatgggggcacggcgcgtggatggaagggcgggcgacgacgacgagggggagaggatcacggggcgaggcataggcgagggtacggcgggggctggccgagattggcggtcgtggccagcgcgggatccgcggcgggggcgagatccggggcgggggcgatggggggacggtgcctgtgcggtcgtggggggaggggcacggcaggggctggcgggctgcttccttccgcgcgcgtgggagggtgcgggggcaggcgggggcaaccaggtgtggacgctgacggtactattttatagagtagtatagatgtgtaggatgcaaaggttcttcgaaggaactttattattgaactgttaagttacgaggaaaattcgtgcagatatgtcatcccgacaaatatacaacagagagttaacaatattattaaaaaagattagattagtgtacaaggtgtcaacatatttatttatgattaagcattatatatttactctttataaactatttgtgtgattccatttcaatctatttgggtagacttatttatatcgttctatatctatgtttaacaatcacatattttgccgtcactattgtactcacgccgcacgtcgtcagggccctaagtctatgaaaaggtcgcaatttgtattccgtggcatcgcacgggcacgttcctagtactacaacaacaacagtactgcccaattcatccattttcacgttccatcttcggcgtgtgacaactcgatcggccacgcacgcagacgacgacggagcagtacttcacagaatcctccgccactcgtcacaccaacaggcgcgcgctggtgcgcatgcatcatgcatgccatcgtccgtcccttggcgtgcctcggtagacggtagctagagtagtagcctgtgcttgctacccctggtcaacacatcgtagcctcctatatttaacgtatcctcacacatcacaagaacgacacacacaaaccagtagccactactccatccatccaccacgagcgcgcgataaccctagctag

SEQ ID NO: 36 - DNA, 921 bp, Zea mays
gcttggtcgccgtagcagcacttgcagaaagaccaagggagcataaggaggcaaaatattttggctgccattgttggtacactattgtgctttctatgttgctagttggtctcaataatatgtgcgatatattagctactaatttaagagatatttatacacatctcagtccttgtgcttgtgcaaggttaattttggattttgcaaaaagaatattagtaagataaggtagagatgacatggtatctagttggttttgacatttagtgattactttgttgggttataatttatacgtaagcaagacaaacttgataaaatacaataaataagtggtttttcctctcggtattgtccacatacatgggtgacaatggtgtgatgcaatacctttacacatgtgtaacgatagcccacatatgtgttgcttggtctggctattttccactagaaaaaaaatcatggcaccattcaccattgttaattttagtgtggttaactttgtgttaataatcggtttaaagaatcactcgatccccaccgatagaaatttaggtacttttagtttcattttaatctataaagttgatactgattttttgatgccaacataaaggcgcctatgtgatttaaacataatgaatatgagcaaaataaatagtaacatataaggaagacattgctagttgatatagtcgtactggttgtaatagtgcagtgttgttgctatgtcgactcaatcacaacaaaaaacactaggaagaagcggaggaagtaaggagttgtgtcgctgatatttccctagaacacgattgtcgtccttcatctgagtagatattcatttcggaggcctgctcagtgctgtaatggagtaccagcccgggccgtcgaccacgcgtgccctatagtgagtcgtattac

SEQ ID NO: 37 - DNA, 960 bp, Zea mays
tcaaaccggtcatcgtttgtatcatccactgcttgacttgggaagaagttaagaacttggtaagacagctgtgagggtgtgacccaactaacccataaatacattttctccaattagtaaattagtttttttttcttggcttgattgatgaatcattcaagttggcatgataagattttgttcagttattcgtgtgtcttatgtatgaaaagtgattgaaaaaaattatggatagttttgacttgctatggatttaattacacctaatcgcctccaatccatatggattggagggaaccaaacaagctctaaggttgatatccgcttctatatatgctgcatgagcagtttactgctttatttttctacagatgggtcagtgatgaggattggtgaatgcatcaggtcattcaaataaatttttttaacgacagggttatgtaggtgatgacacaccatatattccctaactgcctgtctagtgtctactaattactaacgggaaaattgcgtatgctcattgacgtctcagctgtgcagaagaatctcggaacatttaattcacatatattgatactacgtgctagctggtgccatcttcctagctggatactacttattgcatcaattaatttctttttttgttttctttcaattgcttccaaggtcaaactgaatgcaaaccattacttgttacaacggtcctctccatcctacgctacgcctgatgtgatgtaatgtaatcgaagcaagagccttattattgtatatttctgttcctaccagggcttgcatggaaaactgccagcctctcattatattataaatatacgtatactgatacacatacatgcacaccaaaagtactcaggactgtcatctctcagttgcaattgcaaaaaaaatacagagagagagagagagagagagagagatccctaccctgcaaagatatcgacc

SEQ ID NO: 38 - DNA, 401 bp, Zea mays
ggggagacccattaccaatgccaagaacaagaaaccagagaggacagccgcgaatacaatccgaatcccctccaaaaacccaccataccgtctcccccatctgcaagaacaagatcaggagagcatcctagggggaagaaacaggagggggaaaaagaagcccagaaagaccaactgaccaaacaaaaaggtggcatctttgtgtcaccgaggcaccgccgatcacagaccaaagcccgctcctctggtccatcccaagcgtacagcagccgagatttcttcgatctgcgcttgcgtttttttttcttgtaaagattttagttttccccccttcaactgttgctagccttgagtcgctgagaagtttcgagaagggtggtgtgagtgtcggatagctcaag

SEQ ID NO: 39 - DNA, 896 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 40 - DNA, 809 bp, Arabidopsis thaliana
ggttgcatctttgaatacctttttctcatttaggcataacaatataataatttgttttttgttttcattttcttttggtgtcatcttcaaaaatctgtaaacccaaaagtttgtataacttgtttattaagatatttttaattaaatttttttttttgacatttttaaaaaattataaagtgttttatgaatttaaggagtaaataatatttatttagaacactataaattagttttacaagttcttagaaatgtatctgtaaatttcaaaaaggaaaaatatagcatttaattttgaagatttttttctacattatatatatgataaaaatattgtattttgtactttgtagttacaaaaagtcattatatcaacaaatctaaatataaaatatttttctatatattactccaaattaactgtcagaataaaaaagaagaataattattacagaatctgaacattaaaatcgtccctccatatgtggtctctgtctagtccaaaagcaatttacacatcccaagccgaaactatattaaataaacatttttttttctttaactaaaacatttataacatttaacaataaaagttaaaaatcgaacacgtataacgtatttttttacgtatacgtcttgttggcatatatgcttaaaaacttcattacatacatatacaagtatgtctatatatatgatattatgcaaacacaaatctgttgactataattagacttcttcatttactctctctctgacttaaaacatttattttatcttcttcttgttctctctttctctttctctca

SEQ ID NO: 41 - DNA, 2170 bp, Lesquerella fendleri
tcaggaagattaagtctttgcttgttgtctgattttctttaaatacattaagaaatcggttatgaagcttcgttttttgtgttttgggattatgaagctgtctttggatattagttgcggttattagcatgcttctcttttgtgttttggggatgatgaagcagggtctctctatgtaatgcattttgtttgaaaactcagctaatgctaatgcaatttcttttgaaacctttgttatgttttcaaaaatattgaataggttctgttatggatttatttgcaaaagccattgattaaatcaaaccattacataagaacaacattcattattaactaattagagatgcaaaacacaacattacatacaacatcagtgactaattattgagacaaaacaacatcacagacacaaacattcatctcatacatcacttagagagacacaaaaagcaaccaaacacaactattccgccaacaacaattagcttcatacgttttgcttctcctttcaagccttcaatcatcttctcacagccacgaatctgagccttcaataataacatttcttcatcgtgacacttctcacggttatgaatgcaagcctttatgtcctctacttcttctactaaagacacatcggtccacttccaggtgtggaatcctcctcttttgaaatttttctcacaggtatggaataatctacctgggttttttggagttcttgaggttctgatcacaacacggcatccacatcgacaggtcttaggaaaaccacgaaggttatgatcttcaagctcactgtcaaaagataaaaacgagtttgaagaagaagaaggcattatcaatttcagagaattttggagaattttgagagattgagaattgggaaataagaaccctaatccccaatttatgagattgaaaatatatccgttagagaagaaacataatgttgtgcgttttaattagaaaaaatagagatgggctttatcttttgttaagagttttgggcttgggcttgggtttttgataaaaaaattaattaaaccaaaacgacgtcgtttggtttaattgttgttaaaaaaaaattaaaacaccaaaacgacgtcgttttggtgttattaacggccttaaaacggattaaatccataatccgtcagtcaactagggttacggatggtcaacggcgtttttgcataacggaggcacagttcaggcttaacggagtggacggaatggctttttaggaagtttgtaaccggggtcttttgtttatgatgtatttgtccccgtcggctattgttcaggccgtttaggcctttttcctatatactggaaataactattgtccagacgagttacttctccaacatatcaagaagtgttacaaagatgtgttacgaagccataaaactcaaaaccctaagcctaaaccctagaactttctagcacgtttataccttctcctttctttagtttcctttaaaggccttcgtatcataagttttatttttgcttaatactaacactagaaaaaaacaataatcaacataaactaggttaagtcgtggatctaattttattgtgaaaatgtaattgcttctcttaagaaaagattcatagcaaaatattcgcatctttcttgtgaatcatcttttgtttttggggctattaaagaaaaattgaactcatgaaatggtgacaactttattctagaggtaacagaacaaaaatataggaacaacacgtgttgttcataaactacacgtataatactcaagaagatgaatctttataagaatttagttttctcatgaaaacataaaaagttttgtcaattgaaagtgacagttgaagcaaaggaacaaaaggatggttggtgatgatgctgaaatgaaaatgtgtcattcatcaaatactaaatactacattacttgtcactgcctacttctcctctttcctccgccacccattttggacccacgagccttccatttaaaccctctctcgtgctattcaccagaatagaagccaagagagagagagagattgtgctgaggatcattgtcttcttcatcgttattaacgtaagtttttttttgaccacttatatctaaaatctagtacatgcaatagattaatgactgttccttcttttgatattttcagc

SEQ ID NO: 42 - DNA, 1069 bp, Zea mays
gagtcaggtcaacttggccaaacttaaactgtctcggctcgatatattaaggaaccgaataagagaaccaactcgtctcatttgtgagttcgagctctcccgagctaaaaaaacaatatacacatatataaaatagtataaccaattattagttaattctagacctatttaacactaaaaaagagtaacaatactcacactttcacatatcatgtcaatataacaccaaattaacaaatcacttattaattcatccaacacaagtgcgagatttgtttttctgacaaatggttgctcattcaagctaaagagctgactcgaacacagctcgagctggcttgttaacaaatcttgctgagatactagctcagctcgtgacaaaatcaaaatgagctgagctgaattgagtcgagctaaccatgaaccgagcgatctcacgagccacgagtattttgtctagtcctaccaaaaagaccggtccattcttctagtactagtccgaaccccgaaaactttatgatttccatagcatttgtcaaggctgcctcattaatcattttgttgacgatctagagtactctagcgaaaacatgcaagcaaccaaaccgtagagaagtgtagtaggcaaggctggtcgctaatgcgtgcacctggacagtcgtaatcggactgtgcgtgaacgaactaaaaaggcgcaacaaactgttggagtcgctagtcagtacaaaactgaagcggcctttgcccttttgtgactgtcagcacaagcaacaagtccaaactgttgagcacacgatccatccaagtcgacatcctactgctgatcgatcgcgagcttgtcaggttcttcccatccaacgtgcacagctcctatcacggcaaagcaaagcaccagcagcgtacgaggacaaggcctgaatttgttcccaggtgcaacaaacactttttgttctttttagctttgcatccttctcgttccacttacttaatggcacaccatcagcaatgcacaccacggcaacagcattcactgccaagagagtgagcgagcgagcagaggcagcgctgc

SEQ ID NO: 43 - DNA, 2168 bp, Zea mays
ctgcagcctcctctgatatgcatgcctagttactagttgggctaaagtaacccaatttggttatggttacctagctatatagttagttagttagctatcaagtagttccaacacttgcatacaaaaaatagccctcctctagtttaaatgccccaaagctaatttaggttagttttgagctacctagcaattagtacgatccaaacaggctctaagcctattattgatatatatacacataattaagatgggctattggcagtgcaggggatctaggcttgattggcaggtggtgaatgaggtctaggagagtattaagcaatgcaagtgtctctatatatgcatgggggcagctcgcgtacttggtggtagagagggcaccgaccgtcagaagaagaacctaagggtcaaccacagacgacgagagtttatatcgtcattaggttttacaatgtcgtttgcagcttcagcgcgccttggaaaactcggcaagcgataatttcgttgcaacttgataaaaaaatgctatggaagttcatcggatgccttattagcttgagaaaactatttgctatactatactatttctgtttaaattcttagcttacaaatttaattaatgcactaccggaaacgagtgctttgccgagtgtccgaagtactcggcaaagccttaaaaacactcgacaaaggttttaccgagtgtcacactcggcaaagaaggctcggcaaacagtgcatcgtcaaagccttttttgccgagtactttttctcgggcactcggcaaagctttctcgggcactcggcaaacaatacatgtttgccgagtgtttgacactcggcaaacatgtctttgccgattgtggtcatgtgctgagtgtcctgcgctcggtaaatcagctcgttatcgagagccgtactttgccgagtgcggctatcggcaaagccttctttgccgagtgcccgacaaaaggcactcgacaaagagtccgacactcggcaaagcctcggatttcgatagtaatggtgaaattataaatagaatgttataggattcacttaaagaacaaaaaaagtatcttcattatttctttatgtagtaatccgtatacttgctttgcctataaattagagtgtattctcgcttcctgataagttcaaatatttttttgacattgaccaaacatatacaaaacattattaatatttctgataaataataagtatcgttgcattaatagttacttatttgaatatgtaaacattaccaatatttttttgaacttcaccaaacaagttacagccaccaaataagttgctttgtaccaaaaaagttgcagccgccagacttaacactgcacaccatcaaaatcaacgtttcagaccgttagataaccaatatcctttactcattcaaattcaatagacaatactatttggattatctctctccctcacgtcctcggcgcccctctccctcactcgcaccaccgttgtcgccgtcggcaccaccgtcaaccccttccctcgcaccgccaccggtattgtcgacctcttccctcatctctacgggatcatagcgttagcacaaactatatgctagtagatttaatgatctagtgtagtcaaattaaaaaaaattagtgagttactttggtaaccaatagcctccttttctagtatctgtacttccctagagcctagacaaggtgcaagaggaaaccaataaacgactcccaaatttttccaaaaggtttgcacagctcccaagtaaaagctcagttttagtacccgtgcaagttccacgtaggaatttaacagcaaatttttttttgattctttaacaacaaataaataataaaattagtccatctagaccctgtacagttcccacacgtttcaaacggacgaaattaataattacctaaaaatgtgcaggtcgtagcaaagtataattaaattaatacgaaagaataggcacggaaatgccggtaccaaagatataaaaaagggtagaggggaatggaagagtccaggagcaagaaacactcagaagctgcccagagctaccacccttcttatccccacccctcctcctcctaccttttctccttcagacctcaaaatctgtgtgtctcctgccgcggctagctgataggaacaagagc

SEQ ID NO: 44 - DNA, 463 bp, Zea mays
aaggtgcaaggattgctggagcgtcaaggatcattggtgtcgacctgaaccccagcagattcgaagaaggtacagtacacacacatgtatatatgtatgatgtatcccttcgatcgaaggcatgccttggtataatcactgagtagtcattttattactttgttttgacaagtcagtagttcatccatttgtcccattttttcagcttggaagtttggttgcactggccttggtctaataactgagtagtcattttattacgttgtttcgacaagtcagtagctcatccatctgtcccattttttcagctaggaagtttggttgcactggccttggactaataactgattagtcattttattacattgtttcgacaagtcagtagctcatccatctgtcccatttttcagctaggaagttcggttgcactgaatttgtgaacccaaaagaccacaacaagccg

SEQ ID NO: 45 - protein, 9 aa, Artificial Sequence
  FLAG epitope
DYKDDDDKG

SEQ ID NO: 46 - protein, 13 aa, Artificial Sequence
  Thrombin cleavage linker site
LQLVPRGSSAGGG

SEQ ID NO: 47 - protein, 9 aa, Artificial Sequence
  hemagglutinin (HA) epitope
YPYDVPDYA

SEQ ID NO: 48 - protein, 11 aa, Artificial Sequence
  poly histidine tag
SAHHHHHHHHH

SEQ ID NO: 49 - DNA, 882 bp, Zea mays
atggaccagtacgagaaggtggagaagatcggggagggcacgtacggggtggtgtacaagggcaaggaccgccacaccaacgagacgatcgcgctcaagaagatccgcctcgagcaggaggacgagggcgtcccctccaccgccatccgcgagatctccctcctcaaggagatgcagcaccgcaacatcgtcaggctgcaggacgtcgtgcacaacgacaagtgcatctacctcgtcttcgagtacctcgacctcgacctcaagaagcacatggactcctccacggacttcaagaaccaccgcatagtcaaatccttcctctaccagattctgcggggcatcgcctactgccactcgcaccgcgtgctccaccgcgacctgaagccgcagaacctgctgattgaccgccgcaacaacctcttgaagctcgcggactttggactggcgagggcgttcggcatccctgtccggacgttcactcatgaggtggtgacgctttggtatagagcgcccgaaatccttctcggtgcaaggcattattccacccctgttgatgtgtggtcagttggttgcattttcgctgaaatggtgaaccagaaggcgctttttcctggcgactctgagatcgatgagctgtttaagattttcagaattttgggcactccaactaaagaaacatggccaggcgttgcttcgttgcccgattacaagtcaactttcccaaagtggccacctgtggatcttgcaacggtggtcccgacactcgaaccgtcgggaatcgatctcctatctaagatgctgcgtcttgatcccagcaagaggatcaccgcgcgcgccgccctcgagcacgactacttcagggacctcgagcacgcctctaga

SEQ ID NO: 50 - DNA, 1179 bp, Artificial Sequence
  ZmCyclin D4- codon optimized
atggctccatcctgctacgatgcagccgcctcgatgctgctctgcgccgaagaacactccagcatactgtggtatgatgaagaggaggaagagctggaggctgtgggccgccgacgtggccgatcgcccggatatggtgacgatttcggagctgatttgttcccaccacagagtgaggaatgcgtggctggtctcgtagagcgcgagcgagaccacatgcccggtccctgttatggcgaccgtctgcgaggcggaggcggatgcttgtgtgtgcgccgcgaggccgtggattggatctggaaggcgtatacacaccataggtttcggcccctgaccgcctacttggcagtaaattacttggaccgcttcctcagcctgagcgaggtcccggacggcaaggattggatgactcagctcttggcggttgcctgcgtctccctggccgcgaagatggaggaaacggcagttccacaatgccttgacctgcaagtcggagacgcgcggtacgtgtttgaagcaaaaaccgtgcagcgtatggagcttctggtcctcaccaccctcaattggcgtatgcatgccgtcacaccctttagttacgtggattacttccttaacaagctctcgaacggcggtagcaccgctccccgatcgtgctggcttctgcagtcggccgaattgattctgcgcgccgccaggggtacaggctgtgtgggtttccgcccttccgaaattgccgccgcagtggccgctgcggttgccggcgatgtcgacgatgcagacggagtcgagaacgcctgttgcgcgcacgtagataaggaacgagttctgcggtgtcaggaggccatcggcagtatggcaagcagtgctgccatcgacggcgacgccacggtgccacctaaatccgcccggcgtaggtcgagtcctgtgccggttccggtgccggtcccacaaagcccggtgggcgttctggatgccgcggcgtgcctgagctaccgctccgaggaggccgcaactgcaactgccacatccgctgcctcgcacggtcccccgggcagcagcagtagcagctccacgagcccagttacgtcgaagcgccgcaagctggcttcccgttgtgatggatcctgcagcgaccgcagtaagcgcgcgcccgctcagtggaccaaggagtaactcgag

SEQ ID NO: 51 - DNA, 1206 bp, Artificial Sequence
  Zea mays
atggactataaagatgacgacgataaaggagctccatcctgctacgatgcagccgcctcgatgctgctctgcgccgaagaacactccagcatactgtggtatgatgaagaggaggaagagctggaggctgtgggccgccgacgtggccgatcgcccggatatggtgacgatttcggagctgatttgttcccaccacagagtgaggaatgcgtggctggtctcgtagagcgcgagcgagaccacatgcccggtccctgttatggcgaccgtctgcgaggcggaggcggatgcttgtgtgtgcgccgcgaggccgtggattggatctggaaggcgtatacacaccataggtttcggcccctgaccgcctacttggcagtaaattacttggaccgcttcctcagcctgagcgaggtcccggacggcaaggattggatgactcagctcttggcggttgcctgcgtctccctggccgcgaagatggaggaaacggcagttccacaatgccttgacctgcaagtcggagacgcgcggtacgtgtttgaagcaaaaaccgtgcagcgtatggagcttctggtcctcaccaccctcaattggcgtatgcatgccgtcacaccctttagttacgtggattacttccttaacaagctctcgaacggcggtagcaccgctccccgatcgtgctggcttctgcagtcggccgaattgattctgcgcgccgccaggggtacaggctgtgtgggtttccgcccttccgaaattgccgccgcagtggccgctgcggttgccggcgatgtcgacgatgcagacggagtcgagaacgcctgttgcgcgcacgtagataaggaacgagttctgcggtgtcaggaggccatcggcagtatggcaagcagtgctgccatcgacggcgacgccacggtgccacctaaatccgcccggcgtaggtcgagtcctgtgccggttccggtgccggtcccacaaagcccggtgggcgttctggatgccgcggcgtgcctgagctaccgctccgaggaggccgcaactgcaactgccacatccgctgcctcgcacggtcccccgggcagcagcagtagcagctccacgagcccagttacgtcgaagcgccgcaagctggcttcccgttgtgatggatcctgcagcgaccgcagtaagcgcgcgcccgctcagtggaccaaggagtaactcgag

SEQ ID NO: 52 - DNA, 915 bp, Artificial Sequence
  Zea mays
atggaacagtacgagaaggtggagaagataggagagggtacctacggtgtggtttacaaagccctggacaaggccacgaatgaaacaatcgcgcttaagaaaattcggttggagcaggaagatgagggcgtgcctagtactgccattcgtgagatctccctcctgaaagaaatgaatcacggcaatattgtacgattgcacgatgtggtgcacagcgagaaaaggatctacttggtgtttgaatacctcgatcttgatctgaagaaattcatggattcctgccctgagtttgcaaagaaccccacccttattaagtcctacctctaccaaattttgcacggcgtcgcgtactgtcattcccatcgcgttctgcatcgtgatctcaaaccgcagaatctccttatagaccgacgcactaacgcccttaagttggccgatttcggtctggctcgcgcttttggcatccccgtgcgaacctttacacatgaggtcgtcacgctgtggtatcgcgcccccgaaatcttgctcggagcccgccagtatagtactcccgttgatgtgtggagcgtcggctgcatattcgcagaaatggtgaatcaaaagccactgttcccgggagatagcgagatcgacgaactgttcaagattttccggatcctgggaacgccaaacgagcagtcgtggcccggcgtatcctgcctgccagacttcaagacagccttccctcggtggcaagctcaggacctggcgaccgtggtaccgaacctggacccggccggcctggacctcctgtcgaagatgttgcgctatgagccaagcaagaggataaccgcgcgccaagcgctggagcacgagtatttcaaggatctggaggtcgttcagagcgctggattggtcccccgtggctccctcgag

SEQ ID NO: 53 - protein, 305 aa, Zea mays
MEQYEKVEKIGEGTYGVVYKALDKATNETIALKKIRLEQEDEGVPSTAIREISLLKEMNHGNIVRLHDVVHSEKRIYLVFEYLDLDLKKFMDSCPEFAKNPTLIKSYLYQILHGVAYCHSHRVLHRDLKPQNLLIDRRTNALKLADFGLARAFGIPVRTFTHEVVTLWYRAPEILLGARQYSTPVDVWSVGCIFAEMVNQKPLFPGDSEIDELFKIFRILGTPNEQSWPGVSCLPDFKTAFPRWQAQDLATVVPNLDPAGLDLLSKMLRYEPSKRITARQALEHEYFKDLEVVQSAGLVPRGSLE

SEQ ID NO: 54 - DNA, 888 bp, Zea mays
ggatccatggaccagtacgagaaggtggagaagatcggggagggcacgtacggggtggtgtacaagggcaaggaccgccacaccaacgagacgatcgcgctcaagaagatccgcctcgagcaggaggacgagggcgtcccctccaccgccatccgcgagatctccctcctcaaggagatgcagcaccgcaacatcgtcaggctgcaggacgtcgtgcacaacgacaagtgcatctacctcgtcttcgagtacctcgacctcgacctcaagaagcacatggactcctccacggacttcaagaaccaccgcatagtcaaatccttcctctaccagattctgcggggcatcgcctactgccactcgcaccgcgtgctccaccgcgacctgaagccgcagaacctgctgattgaccgccgcaacaacctcttgaagctcgcggactttggactggcgagggcgttcggcatccctgtccggacgttcactcatgaggtggtgacgctttggtatagagcgcccgaaatccttctcggtgcaaggcattattccacccctgttgatgtgtggtcagttggttgcattttcgctgaaatggtgaaccagaaggcgctttttcctggcgactctgagatcgatgagctgtttaagattttcagaattttgggcactccaactaaagaaacatggccaggcgttgcttcgttgcccgattacaagtcaactttcccaaagtggccacctgtggatcttgcaacggtggtcccgacactcgaaccgtcgggaatcgatctcctatctaagatgctgcgtcttgatcccagcaagaggatcaccgcgcgcgccgccctcgagcacgactacttcagggacctcgagcacgcctctaga

SEQ ID NO: 55 - protein, 296 aa, Zea mays
GSMDQYEKVEKIGEGTYGVVYKGKDRHTNETIALKKIRLEQEDEGVPSTAIREISLLKEMQHRNIVRLQDVVHNDKCIYLVFEYLDLDLKKHMDSSTDFKNHRIVKSFLYQILRGIAYCHSHRVLHRDLKPQNLLIDRRNNLLKLADFGLARAFGIPVRTFTHEVVTLWYRAPEILLGARHYSTPVDVWSVGCIFAEMVNQKALFPGDSEIDELFKIFRILGTPTKETWPGVASLPDYKSTFPKWPPVDLATVVPTLEPSGIDLLSKMLRLDPSKRITARAALEHDYFRDLEHASR

SEQ ID NO: 56 - protein, 191 aa, Artificial Sequence
  Arabidopsis thaliana
MVRKYRKAKGIVEAGVSSTYMQLRSRRIVYVRSEKSSSVSVVGDNGVSSSCSGSNEYKKKELIHLEEEDKDGDTETSTYRRGTKRKLFENLREEEKEELSKSMENYSSEFESAVKESLDCCCSGRKTMEETVTAEEEEKAKLMTEMPTESEIEDFFVEAEKQLKEKFKKKYNADAEKEKPLEGRYEWVKLE

SEQ ID NO: 57 - protein, 190 aa, Zea mays; identical to SEQ ID NO: 7

SEQ ID NO: 58 - protein, 361 aa, Arabidopsis thaliana
MAENLACGETSESWIIDNDDDDINYGGGFTNEIDYNHQLFAKDDNFGGNGSIPMMGSSSSSLSEDRIKEMLVREIEFCPGTDYVKRLLSGDLDLSVRNQALDWILKVCAHYHFGHLCICLSMNYLDRFLTSYELPKDKDWAAQLLAVSCLSLASKMEETDVPHIVDLQVEDPKFVFEAKTIKRMELLVVTTLNWRLQALTPFSFIDYFVDKISGHVSENLIYRSSRFILNTTKAIEFLDFRPSEIAAAAAVSVSISGETECIDEEKALSSLIYVKQERVKRCLNLMRSLTGEENVRGTSLSQEQARVAVRAVPASPVGVLEATCLSYRSEERTVESCTNSSQSSPDNNNNNNNSNKRRRKQ

SEQ ID NO: 59 - DNA, 885 bp, Arabidopsis thaliana
atggatcagtacgagaaagttgagaagattggtgaaggaacttacggtgtggtttataaggcacgtgacaaagtgactaatgagacaattgctttgaagaagatcaggctagagcaggaggatgaaggtgttcctagcacagcaatcagagaaatctccctcttgaaagaaatgcagcacagcaacattgtcaaattgcaggatgtggtgcacagcgagaaacgtttgtatctggtttttgagtatcttgacttggatctcaaaaagcacatggattctactcctgatttctccaaggatctacatatgatcaaaacatatctttaccagattctccgtggaattgcgtattgccactctcatagggttctccatcgtgatctgaagccacagaatttgttgattgatcgccgcacaaactcactgaagcttgctgattttggactggccagagcattcggtatccctgtcaggacatttactcatgaggttgttactctctggtaccgagcaccagagatactcctaggatctcatcattactctacacctgttgatatttggtctgtggggtgcatatttgctgagatgatcagccaaaagcccttatttcctggagactccgagattgatcaactcttcaagattttcagaatcatgggaactccgtacgaggatacatggcgtggggtaacttctctaccggattataaatctgctttccctaaatggaaaccaacggacctagaaacttttgtccccaatctagatcccgatggagtcgatctcctttctaaaatgctgttaatggatccgaccaaaagaatcaacgcaagagccgccctggagcatgaatacttcaaggatcttggaggcatgccttag

SEQ ID NO: 60 - protein, 294 aa, Arabidopsis thaliana [sequence omitted]

SEQ ID NO: 61 - DNA, 1760 bp, Arabidopsis thaliana
acctcacatattcatcaccatcgtatccacaaacattatcgagacataaataataatttatagatatatagcaacaaattggaattaaattaatcaaacaatgtcatttagaattggggaaatcctaaccgacgttatatgattaattttaattccaatttatgtatgtgtatatatctataaattattatttatatattgatgatgttcgtgcatgatggtaatggtaacctttaaaacttttgaatctcaaaacgtaaaacaacgtcatttggtttgaagaacatgccatctcgacgtacaagtgttagctgattcaacaaccgttactgttaattaccataactaacgttgactcataattatgtacaaaagtaagagttgaggtagataatttgaattttttaaatgttaaagtatgaacattactaattgtttgttcagatttataaaacacagtacctattttgatgtgttaaaatgtaatcgggaaaataggaattttcccaagaaaaaaaaatacactaaaattttgatgtgttaaaatgtaataaatacaagtacctatttctttctacaacaggtatcacatgatctgctcaatctcattttgaaggcaaggtcgaaccctgtttgtaactaaatattttacaaatacactaaaagttttgttttgttttcctcatgcagaaaaaagaaaagaaagaagaagaaaaaaaactcacaaaaacatacattgttctacaatatgttaaagtaattgttctagggtaataaaatccgtgtctgcctcttccacttcaataggaaagtgtaagtttttcgaatctttttggccagataatttaaaaccgaccgaggaaacttacttttgtaaaagtgaccatttgcacataggaagtagaaagttgataattggaaaatttataatattttatacaaaagaagtgtttaagaaccaccgcttggtctcaccactcaaattcgaacgtatggctcgtgggcacaaactcctgtaccaacctttagatactagcggcacgaggagacttctattttctctttgtttttaagtttttttatcgacttaaatatggctttttgcaccatttaatttcaaaaccaatgtctttctctcgaggaactttgtttttatttcttagaagatgaggggagatttactatctaaataaaattttaaatgtttgtaagtattatgagctcaacaattttgtcaatagtgccacaaatttaaacgtttgctttttgtcttctttgaaaaatcaaatgctgaaaaactgttacatctctttttcttaaaaactcttgtctctctccttttccttctgctgaggtaattgaatgctgcaaagagaaagagaataacttaaacccaaaattacacttaccgccagaaaaaaaaaagagttcagtttaatctaacatattttatacaatacaattgaattatattagtaaaaaaaaaacttccatataaatcatggaacaaactggaacacatgggctctcttattttaatttattttcttttttgagggatttaaccatgtttattatatagttttataaatatatatataccatctctccataatttataaaatgaaagaaagagagagtcttcttgttggagtaaacccaaacggttttagattacttattagctgttcatcagttcttcctctctaaaagagtaaaacctaaacatctctctctgttctattagaaccaaagaccaa

SEQ ID NO: 62 - protein, 325 aa, Zea mays
MPGDAYDLLCAEDDAAGSALLCTTDDDDGCCSAVEDDSAAAAAASIAELIGGEAQYSPRPDYPDRLRSRSIDPAARAESVAWILKVQEYYGFLPLTAYLAVNYMDRFLSLHRLPQEDGWAMQLLAVTCLSLAAKMEETLVPSLLDLQVEGTSRYDCFEPGTVGQMELLVLMALNWRLRSVTPFTFVDFFACKVDPGGRHTRCLIARATQVILAAMHDVEFLDHCPSSMAAAAVLCAIGETPSLESVSPGAAVSWCIGLAEEGISSCYRLMQRLVIGNVRTRVASTTTITMAAVNLCCSDEALSSHSSATSSPPPAKRRKRSPTAT

SEQ ID NO: 63 - protein, 324 aa, Zea mays
MPGDAYDLLCAEDDAAGSALLCTTDDDDGCCSAVEDDSAAAAAASIAELIGGEAQYSPRPDYPDRLRSRSIDPAARAESVAWILKVQEYYGFLPLTAYLAVNYMDRFLSLHRLPEDGWAMQLLAVTCLSLAAKMEETLVPSLLDLQVEGTSRYDCFEPGTVGQMELLVLMALNWRLRSVTPFTFVDFFACKVDPGGRHTRCLIARATQVILAAMHDVEFLDHCPSSMAAAAVLCAIGETPSLESVSPGAAVSWCIGLAEEGISSCYRLMQRLVIGNVRTRVASTTTITMAAVNLCCSDEALSSHSSATSSPPPAKRRKRSPTAT

SEQ ID NO: 64 - protein, 177 aa, Zea mays
MKADERRLVVVDCTALQVEGTSRYDCFEPGTVGQMELLVLMALNWRLRSVTPFTFVDFFACKVDPGGRHTRCLIARATQVILAAMHGNIIMHCLCLGLGAAGVNLVLRCSACSSNLQCTNCGTATDVEFLDHCPSSMAAAAVLCAIGETPSLESVSPGAAVSWCIGLAEVYTPHKNY

SEQ ID NO: 65 - protein, 331 aa, Zea mays
MVPGYDCAASVLLCAEDNAAILGLDDDGEESSWAAAATPPRDTVAAAAAAATGVAVDGILTEFPLLSDDCVATLVEKEVEHMPAEGYLQKLQRRHGDLDLAAVRKDAIDWIWKEGRAWMTQLLAVACLSLASKIEETFVPLPLDLQVAEAKFVFEGRTIKRMELLVLSTLKWRMHAVTACSFVEYFLHKLSDHGAPSLLARSRSSDLVLSTAKGAEFVVFRPSEIAASVALAAIGECRSSVIERAASSCKYLDKERVLRCHEMIQEKITAGSIVLKSAGSSISSVPQSPIGVLDAAACLSQQSDDATVGSPAVCYHSSSTSKRRRITRRLL

SEQ ID NO: 66 - protein, 360 aa, Zea mays
MVPGYDCAASVLLCAEDNAAILGLDDDGEESSWAAAATPPRDTVAAAAAAATGVAVDGILTEFPLLSDDCVATLVEKEVEHMPAEGYLQKLQRRHGDLDLAAVRKDAIDWIWKVIEHYNFAPLTAVLSVNYLDRFLSTYEFPEGRAWMTQLLAVACLSLASKIEETFVPLPLDLQVAEAKFVFEGRTIKRMELLVLSTLKWRMHAVTACSFVEYFLHKLSDHGAPSLLARSRSSDLVLSTAKGAEFVVFRPSEIAASVALAAIGECRSSVIERAASSCKYLDKERVLRCHEMIQEKITAGSIVLKSAGSSISSVPQSPIGVLDAAACLSQQSDDATVGSPAVCYHSSSTSKRRRITRRLL

SEQ ID NO: 67 - protein, 345 aa, Zea mays
MGILCLGASSTLLCGEDRNNVLGLGCGNELVEVGSGHDGLDSVVGAVFPVDTDEAVRALLEKETDHKPQDGYAERLERGGLEYSWRRDAMDWICKVHSYYRFGPLSLYLAVNYLDRFLSSYDLPHDKPWMRQLLSVACLALAVKMEETVLPLPVDLQVCDVKFEFEARTIGRMELLVLATLKWRMQAVTPFTFISYFLDKFNGGKPPSLALASRCTDIIIGTLKGSTFLSFRPSEIAAASALAAVSENQVVGSSSALSASEVPINKVMIARCYELLQEQALVRKTGHVNGSPSVPQSPIGVLDATCFSFRSEDARLVSSQSNNISSSSSNDNQVSKRRRLSISPI

SEQ ID NO: 68 - protein, 387 aa, Zea mays
MAAFAALFDPLYCPEEHLDLYHEGPVEVVDEQWQDQRGQQQPAALDDELPALFEALRDKEGVVLAGDGEEDGYGGSAGREAAVGWASRAAARLGFSALTSALSAAYLDRCFLPGGALRLGDQPWMSRLAAVACVALAAKVEETRVPLLLDLQLCAAASSDADAADADVFEAKTVRRMELLVLSALGWRMHPVTPFSYLQPVLADAAMRLRNCEAVLLAVMADWRWPRHRPSAWAAAALLTTAGGGDDDSELLALINAPEDETAECAKIISEVTGMSFLVCDVGGMIAGNKRKHAAARMYSPPLSPSGVIGALSCFSCESSLSATADSRTLATTAAGVGPWAPSAPVSVSSSPEPPGRAPKRAAAAGVPHPLPPDEESRDAWPSTCAA

SEQ ID NO: 69 - protein, 270 aa, Zea mays
MAAFAALFDPLYCPEEHLDLYRDEPGEGADEQWPGQHGQQEPAVLDDELPALFEAHRAKEGVVLAEDGGYGGAAGREAAVGWVSRAAARLGFSALTAALAAAYLDRCFLPGGALRLGDQPWMARLAAVTCFALAAKVEETRVPPLLDLQLYAAADAADPYVFEAKTVRRMELLVLSALGWRMHPVTPFSYLQPVLADAATRLRSCEGVLLAVMADWRWPRHRPSAWAAAALLITAAAGDGGDGDGDTELLALINAPEVSAAQCHCISTIL

SEQ ID NO: 70 - protein, 391 aa, Zea mays
MAAFAALFDPLYCPEEHLDLYRDEPGEGADEQWPGQHGQQEPAVLDDELPALFEAHRAKEGVVLAEDGGYGGAAGREAAVGWVSRAAARLGFSALTAALAAAYLDRCFLPGGALRLGDQPWMARLAAVTCFALAAKVEETRVPPLLDLQLYAAADAADPYVFEAKTVRRMELLVLSALGWRMHPVTPFSYLQPVLADAATRLRSCEGVLLAVMADWRWPRHRPSAWAAAALLITAAAGDGGDGDGDTELLALINAPEDKTAECAKIISEVTGMSFLACDVGVSAGNKRKHAAAQLYSPPPSPSGVIGALSCFSCESSTSATAMAAAVGPWAPSASVSVSSSPEPPGRAPKRAAAASASASASAGVAPPVQVPHQLPPDEESRDAWPSTCAA

SEQ ID NO: 71 - protein, 361 aa, Zea mays
MAPSSYEMAASTLLCGEDSSSILDLEAGGQEEEEEVLLARSRTRGEPSVVFPVPSEECVAGFVEAEAAHMPREDYAERLRGGGTDLRVRTDAIDWIWKVHAYYGFGPLTACLAVNYLDRFLSLYQLPEGKSWTTQLLSVACLSLAAKMEETYVPPSLDLQVGDARYVFEAKTVQRMELLVLSTLRWRMRAVTPFSYIDYFLHRLKDGGAPSRRAVLRSAELILRVARGTCCLGFRPSEVAAAVAAAVAGEEHAVDIDKACTHRVHEERVSRCLEAIQATVALLAPAQPLKAEGPSSGRSRASSSSATVPRSPTGVLDAGCLSYRSDDAAAAAAASHASSAWRDEDGDCSPAVCSKRRKTSR

SEQ ID NO: 72 - protein, 388 aa, Zea mays
MAPSCYDAAASMLLCAEEHSSILWYDEEEEELEAVGRRRGRSPGYGDDFGADLFPPQSEECVAGLVERERDHMPGPCYGDRLRGGGGCLCVRREAVDWIWKAYTHHRFRPLTAYLAVNYLDRFLSLSEVPDCKDWMTQLLAVACVSLAAKMEETAVPQCLDLQEVGDARYVFEAKTVQRMELLVLTTLNWRMHAVTPFSYVDYFLNKLNNGGSTAPRSCWLLQSAELILRAARGTGCVGFRPSEIAAAVAAAVAGDVDDADGVENACCAHVDKERVLRCQEAIGSMASSAAIDDATVPPKSARRRSSPVPVPQSPVGVLDAAPCLSYRSEEAATATATATSAASHGAPGSSSSSSTSPVTSKRRKLASRCDGSCSDRSKRAPAQWTKE

SEQ ID NO: 73 - protein, 355 aa, Zea mays
MAPSSYEVAASILLCAEDSSSILDLEAEAEEEALLARSGEPGGGAEFPVPSEECVAGFLESEAAHMPREDYAERLRSGGMDLRVRTDAIDWIWKVHTCYGFGPLTACLAVNYLDRFLSLYQLPEGKAWMTQLLSVACLSLAAKMEETYVPSSLDLQAGDARYVFEAKTIQRMELLVLSTLKWRMQAVTPLSYVDYFLHRLRGGAAPSRRAVLRSAELILCIARGTHCLDFRPSEIALAVAATVAGEERAVDIDRAFTHRVHKERVSRCLEAIQQATATMALPQPLKSEGPSSSGRRASSSSATVPRSPTGVLDAGCLSYRRAAAAAASHASSSWRDEDDDDCPPAVCSKRRKISR

(19) United States
(12) Patent Application Publication
(10) Pub. No.: US 2012/0131698 A1
(43) Pub. Date: May 24, 2012

(54) Dominant Negative Mutant Kip-Related Proteins (KRP) in Zea Mays and Methods of Their Use

(75) Inventors: Jean Paul Olivier, Seattle, WA (US); James Michael Roberts, Seattle, WA (US)

(73) Assignee: TARGETD GROWTH, INC., Seattle, WA (US)

(21) Appl. No.: 13/295,809

(22) Filed: Nov. 14, 2011

Related U.S. Application Data
(60) Provisional application No. 61/413,004, filed on Nov. 12, 2010.

(51) Int. Cl. C12N 15/82; C12N 5/10; A01H 5/08; A01H 5/00; A01H 5/10

(52) U.S. Cl. 800/290; 435/320.1; 800/278; 800/298; 800/320; 800/320.1; 800/320.2; 800/320.3; 435/419

(57) ABSTRACT

The present invention provides expression vectors comprising polynucleotides encoding mutant Zea mays KRP dominant negative proteins, and methods of using the same. In addition, transgenic plants expressing said KRP dominant negative proteins are provided. Furthermore, methods of increasing average seed weight, seed size, seed number and/or yield of a plant by using said KRP dominant negative proteins are provided.

### CROSS-REFERENCE TO RELATED APPLICATIONS

This application claims the benefit of U.S. Provisional Patent Application Ser. No. 61/413,004, filed Nov. 12, 2011, which is hereby incorporated by reference in its entirety for all purposes.

### TECHNICAL FIELD

The invention generally relates to methods for increasing crop yield. More specifically, the present invention relates to methods and compositions for increasing plant seed weight, seed size, seed number and/or yield by expressing one or more dominant negative Kinase Inhibitor Protein (KIP) Related Proteins (KRP) in the plant.

### BACKGROUND

The most important trait as a target for crop improvement is yield. Efforts to improve crop yields by developing new plant varieties can be divided into two approaches. One is to reduce crop yield losses by breeding or engineering crop varieties with increased resistance to abiotic stress conditions such as drought, cold, or salt or to biotic stress conditions resulting from pests or disease-causing pathogens. While this approach has value, it does not provide fundamentally improved crop yield in the absence of stress conditions and in fact, such resistance may direct plant resources that otherwise would be available for increased yield in the plant. The second approach is to breed or engineer new crop varieties in which the basic yield capacity is increased.

Classical breeding programs have initially produced substantial gains in improved yield in a variety of crops. A commonly experienced pattern though has been substantial gains in yield initially followed by incremental further improvements that become smaller and more difficult to obtain. More recently developed approaches based on molecular biology technologies have in principle offered the potential to achieve substantial improvement in crop yield by altering the timing, location, or level of expression of plant genes or heterologous genes that play a role in plant growth and/or development. Substantial progress has been made over the past twenty years in identifying plant genes and or heterologous genes that have a role in plant growth and/or development. Because of the complexity of plant growth regulation and how it relates in the end to yield traits, it is still not obvious which, if any, of these genes would be a clear candidate to improve crop yield.

Previously dominant negative mutant Kinase Inhibitor Protein (KIP) Related Protein (KRP) derived from Brassica napus (BnKRP1) and Arabidopsis thaliana (AtKRP1) were described, see, International Patent Application Publication No. WO2007016319, which is incorporated by reference in its entirety for all purposes. It was shown in in vitro assays that dominant negative (DN) mutant KRP proteins could be engineered to compete with wild-type KRP proteins to effectively bind to Cyclin/CDK complexes. The binding of the mutant KRP DN proteins, in contrast to the wild-type KRP proteins, allows the kinase complex to maintain its enzymatic phosphorylation activity. This protection of Cyclin/CDK kinase activity by mutant KRP DN proteins allows progression of the cell cycle for cell division.

The in vitro assays in the KRP DN of WO2007016319 used Arabidopsis cyclin D2;1 and Arabidopsis CDKA. The tested KRP DN proteins were from Arabidopsis thaliana and Brassica napus. Two BnKRP1 DN mutants (BnKRP1 DN#2 [F151A;F153A], SEQ ID NO: 3 and BnKRP1 DN#3 [Y149A;F151A;F153A], SEQ ID NO: 4, both of which are based on wild-type BnKRP1, SEQ ID NO: 2) exhibited strong dominant negative activity in these assays. The mutations equivalent to the ones in BnKRP1 DN#2 were introduced into AtKRP1, and the AtKRP1 DN#2 (F173A;F175A), driven by an embryo-specific promoter (LFAH12) was introduced into Brassica napus (canola) plants. Field trials indicated that canola plants homozygous for the LFAH12-AtKRP1 DN#2 transgene had seed yield increases compared to the null sibling plants that did not contain the transgene.

The inventors of the present invention have unexpectedly discovered that the Arabidopsis thaliana and Brassica napus KRP dominant negative mutants taught in the prior art are not useful in protecting the Cyclin/CDK complex from inhibition by a wild-type KRP protein in Zea mays (aka maize or corn), a monocotyledenous plant. Furthermore, the present invention demonstrates that not all Zea mays KRP DN (ZmKRP DN) mutants work in maize, but that specific ones are useful in protecting specific Cyclin/CDK complexes from inhibition by a wild-type KRP protein in Zea mays.

### SUMMARY

The present invention provides compositions and methods for interfering with wild-type Zea mays KRP activity using levels of Dominant Negative KRP (“DN KRP”) expression that are physiologically achievable in maize.

The present invention provides recombinant polynucleotides having a nucleic acid sequence encoding a mutant KRP, wherein the mutant KRP comprises amino acid sequence having at least one modification relative to a wild-type KRP, biologically active variant, or fragment thereof, said wild-type KRP polypeptide comprises (a) a cyclin binding region conferring binding affinity for a cyclin and (b) a cyclin-dependent kinase (CDK) binding region conferring binding affinity for a CDK. In some embodiments, the cyclin and the CDK can form a complex. In some embodiments, the wild-type KRP has at least 47% identity to Zea mays KRP1 (ZmKRP1) or KRP2 (ZmKRP2). In some embodiments, the mutant KRP polypeptide does not inhibit, or does not substantially inhibit kinase activity of the Cyclin/CDK complex. In some other embodiments, the mutant KRP polypeptide is capable of increasing seed size, seed weight, and or yield when expressed in a plant, for example, in a Zea mays plant. In some further embodiments, the mutant KRP can compete with one or more wild-type Zea mays KRPs for binding to the CDK binding region. In some embodiments, optionally, the polynucleotide is operably-linked to a plant promoter. In some embodiments, the nucleic acid sequence when incorporated into a plant leads to increased seed number, seed size, and/or yield of the plant. For example, in some embodiments, the expression vector comprises a polynucleotide having a nucleic acid sequence encoding a mutant KRP, wherein the mutant KRP comprises an amino acid sequence having at least one modification relative to a wild-type KRP, biologically active variant, or fragment thereof, said wild-type KRP polypeptide comprising (a) a cyclin binding region conferring binding affinity for a cyclin, and (b) a cyclin-dependent kinase (CDK) binding region conferring binding affinity for a CDK, wherein the cyclin and the CDK can form a complex; wherein the wild-type KRP has at least 47% identity to Zea mays KRP1 (ZmKRP1) or KRP2 (ZmKRP2); and wherein the mutant KRP polypeptide is capable of increasing seed size, seed weight, and or yield when expressed in a Zea mays plant. In some further embodiments, the mutant KRP polypeptide can compete with one or more wild-type Zea mays KRPs for binding to the CDK binding region; and optionally, the polynucleotide is operably-linked to a plant promoter.

In some embodiments, the mutant KRP is derived from a wild-type KRP in Zea mays (ZmKRP), or biologically active variant, or fragment thereof. In some embodiments, said Zea mays KRP (ZmKRP) is Zea mays KRP1, SEQ ID NO: 7, or ZmKRP2, SEQ ID NO: 11, biologically active variant, or fragment thereof. In other embodiments, said mutant KRP is derived from a wild-type KRP from species other than Zea mays, wherein the wild-type KRP shares at least 47% identity to ZmKRP1 or ZmKRP2.

In some embodiments, the mutant KRP protects one or more Zea mays Cyclin/CDK complexes from one or more wild-type Zea mays KRPs. In some other embodiments, the mutant KRP protects one or more Cyclin/CDK complexes from one or more wild-type KRPs, wherein the Cyclin/CDK complexes and the wild-type KRPs are derived from a species other than Zea mays. In some embodiments, said mutant KRP proteins when expressed in a plant increase seed weight, see size, and/or yield of that plant. In some embodiments, said plant is monocotyledonous, for example, Zea mays.

In some embodiments, the mutant KRP is derived from ZmKRP1, or biologically active variant, or fragment thereof, wherein the mutant KRP has at least two modifications at the positions relative to amino acid position 234 and position 236 of the wild-type Zea mays KRP2, e.g., at position 172 and position 174 of ZmKRP1 (SEQ ID NO: 7). In some embodiments, the two modifications are F172A and P174A relative to the wild-type ZmKRP1 (SEQ ID NO: 8). In some other embodiments, the two modifications are F172Xaa₁ and F174Xaa₂, wherein Xaa₁ is any amino acid other than phenylalanine (Phe or F), and Xaa₂ is any amino acid other than proline (Pro or P).

In some embodiments, the mutant KRP is derived from ZmKRP2, or biologically active variant, or fragment thereof, wherein the mutant KRP has at least two modifications relative to the wild-type Zea mays KRP2 at amino acid position 234 and position 236. In some embodiments, the two modifications are F234A and F236A relative to the wild-type ZmKRP2 (SEQ ID NO: 12). In some other embodiments, the two modifications are F234Xaa₁ and F236Xaa₂, wherein Xaa₁ and Xaa₂ are any amino acids other than phenylalanine (Phe or F).

In some embodiments, the Cyclin/CDK complexes comprise a CDK protein selected from the group consisting of Zea mays CDK A;1 (ZmCDKA;1, SEQ ID NO: 53), Zea mays CDK A;2 (ZmCDKA;2, SEQ ID NO: 55), and a cyclin protein selected from the group consisting of Zea mays Cyclin D1, D2, D3, D4, D5, D6, D7, and combinations thereof, and the wild-type Zea mays KRPs are selected from the group consisting of ZmKRP1, ZmKRP2, ZmKRP3, ZmKRP4, ZmKRP5, ZmKRP6, ZmKRP7, ZmKRP8, and combinations thereof. For example, the wild-type Zea mays KRP is ZmKRP1, ZmKRP2, or ZmKRP5.

The present invention also provides expression vectors comprising the recombinant polynucleotides of the present invention. In some embodiments, the polynucleotide sequence is codon-optimized for expression in certain cell types, for example, expression in bacteria cell, insect cell, or plant cell.

In some embodiments, the expression vectors comprise a promoter. In some embodiments, the polynucleotide encoding the mutant KRP is operably-linked to a promoter. In some embodiments, the promoter is a plant promoter. In some further embodiments, the plant promoter is a constitutive promoter, a non-constitutive promoter, an inducible promoter, or a tissue or organ specific promoter. In some embodiments, the plant promoter is an embryo-specific promoter, an endosperm-specific promoter, or an ear-specific promoter. In some embodiments, the plant promoter is a promoter selected from the group consisting of promoters associated with ZmOleosin gene, Hordeum vulgare PER1 (HvPER1) gene, END2 gene (e.g., U.S. Pat. No. 6,528,704), LEC1 gene (e.g., U.S. Pat. No. 7,166,765), zein genes (e.g., CZ19B1 gene), EEP1 gene (e.g., U.S. Pat. No. 7,803,990), PP1A gene (e.g., Smith et al. 1991, Plant Physiol. (1991) 97, 677-683.), ABI3 gene (e.g., Arabidopsis ABI3 gene or maize VP1 gene) and Ubiquitin gene (e.g., U.S. Pat. No. 5,510,474). In some embodiments, the promoters mentioned above are associated with ZmEND2, ZmLEC1, ZmZein, ZmEEP1, ZmPP1A, ZmVP1, or ZmUbiquitin genes. In some other embodiments, the promoters mentioned above are associated with END2, LEC1, Zein, EEP1, PPM, ABI3, or Ubiquitin genes of a plant species other than Zea mays, for example, a monocot or a dicot plant other than Zea mays.

In some embodiments, the expression vectors further comprise an enhancer sequence. In some embodiments, the enhancer sequence is an intron-mediated enhancement (IME) element, and wherein the IME element is between the plant promoter and the polynucleotide encoding the mutant KRP. In some further embodiments, the IME element is the first intron of maize ADH1 gene, or functional variants or fragments thereof.

The present invention also provides methods for increasing average seed size, seed weight, and/or yield in a plant. In some embodiments, the methods comprise incorporating into the plant the recombinant polynucleotides of the present invention. In some embodiments, the recombinant polynucleotides are nucleic acid sequences encoding a mutant KRP comprising amino acid sequence having at least one modification relative to a wild-type KRP, biologically active variant, or fragment thereof, said wild-type KRP polypeptide comprises (a) a cyclin binding region conferring binding affinity for a cyclin and (b) a cyclin-dependent kinase (CDK) binding region conferring binding affinity for a CDK, wherein the wild-type KRP has at least 47% identity to Zea mays KRP1 (ZmKRP1) or KRP2 (ZmKRP2); wherein the cyclin and the CDK form a complex; wherein the mutant KRP polypeptide does not inhibit kinase activity of the Cyclin/CDK complex; wherein the mutant KRP polypeptide can compete with one or more wild-type Zea mays KRPs for binding to the CDK binding region; and optionally, the polynucleotide is operably-linked to a plant promoter. In some embodiments, the Cyclin/CDK complexes comprise a CDK protein selected from the group consisting of Zea mays CDK A;1 (ZmCDKA;1, SEQ ID NO: 53), Zea mays CDK A;2 (ZmCDKA;2, SEQ ID NO: 55), and a cyclin protein selected from the group consisting of Zea mays Cyclin D1, D2, D3, D4, D5, D6, D7, and combinations thereof, and the wild-type Zea mays KRPs are selected from the group consisting of ZmKRP1, ZmKRP2, ZmKRP3, ZmKRP4, ZmKRP5, ZmKRP6, ZmKRP7, ZmKRP8, and combinations thereof. In some embodiments, the wild-type KRP is ZmKRP1 or ZmKRP2. In some further embodiments, the mutant KRP comprises at least two modifications relative to the wild-type Zea mays KRP2 at amino acid position 234 and position 236. In some embodiments, the two modifications are F234A and F236A relative to the wild-type ZmKRP2 (SEQ ID NO: 12). In some other embodiments, the two modifications are F234Xaa₁ and F236Xaa₂, wherein Xaa₁ and Xaa₂ are any amino acids other than phenylalanine (Phe or F). In some further embodiments, the mutant KRP comprises at least two modifications relative to the wild-type Zea mays KRP1 at amino acid position 172 and position 174. In some other embodiments, the two modifications are F172Xaa₁ and F174Xaa₂, wherein Xaa₁ is any amino acid other than phenylalanine (Phe or F), and Xaa₂ is any amino acid other than proline (Pro or P).

In some embodiments, the methods increase the seed size, seed weight, and/or yield of the plant by at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2.0%, at least 2.1%, at least 2.2%, at least 2.3%, at least 2.4%, at least 2.5%, at least 2.6%, at least 2.7%, at least 2.8%, at least 2.9%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 120%, at least 140%, at least 160%, at least 180%, at least 200%, or more compared to a wild-type or control plant not expressing the mutant KRP.

The present invention further provides transgenic plants expressing the polynucleotides of the present invention as described herein. In some embodiments, the transgenic plant is a dicotyledonous plant or a monocotyledonous plant. For example, the transgenic plant can be a monocotyledon plant selected from the group consisting of corn, rice, wheat, barley, sorghum, millets, oats, ryes, triticales, buckwheats, fonio, quinoa and oil palm.

The present invention further provides a seed, a fruit, a plant cell or a plant part of the transgenic plants as described herein. For example, the present invention provides a pollen of the transgenic plant, an ovule of the transgenic plant, a genetically related plant population comprising the transgenic plant, a tissue culture of regenerable cells of the transgenic plant. In some embodiments, the regenerable cells are derived from embryos, protoplasts, meristematic cells, callus, pollen, leaves, anthers, stems, petioles, roots, root tips, fruits, seeds, flowers, cotyledons, and/or hypocotyls.

### BRIEF DESCRIPTION OF THE DRAWINGS

FIG. 1 depicts percent recovery of AtCyclin D2;1/AtCDKA kinase function in presence of the wild-type Zm KRP4 alone or with BnKRP1 DN#2 or BnKRP1 DN#3.

FIG. 2 depicts autoradiograph of kinase assays using ZmCyclinD4/CDKA;1 kinase complex, wild-type Zm KRPs and indicated Brassica napus (Bn) or Zea mays (Zm) dominant negative (DN) KRPs. Histone H1 (HH1) was used as the substrate for phosphorylation. Lanes 1, 5, 9, 13, 17, 22, 26 and 30 contain just the kinase complex without any wild-type or dominant negative KRPs. Lane 18 contains just the kinase complex in buffer. Lanes 2, 6, 10, 14, 19, 23, 27 and 31 contain the kinase complex, wild-type ZmKRP1, and the indicated Zm or Bn KRP DN (above the lanes). Lanes 3, 7, 11, 15, 20, 24, 28 and 32 contain the kinase complex, wild-type ZmKRP2, and the indicated Zm or Bn KRP DN. Lanes 4, 8, 12, 16, 21, 25, 29 and 33 contain the kinase complex, wild-type ZmKRP5, and the indicated Zm or Bn KRP DN.

FIG. 3 depicts percent recovery of ZmCyclinD4/ZmCDKA;1 kinase function in presence of the indicated wild-type Zm KRP alone or with BnKRP1DN#2 or BnKRP1DN#3.

FIG. 4 depicts percent recovery of ZmCyclinD4/ZmCDKA;1 kinase function in presence of the indicated wild-type Zm KRP alone or with ZmKRP2DN#2. This experiment quantifies the ability of ZmKRP2 DN#2 to compete with wild-type ZmKRPs 1, 2 or 5 to protect corn-specific ZmCyclinD4/ZmCDKA;1 kinase complex.

FIG. 5 depicts percent recovery of ZmCyclinD4/ZmCDKA;2 kinase function in presence of the indicated wild-type Zm KRP alone or with ZmKRP2DN#2. This experiment quantifies the ability of ZmKRP2 DN#2 to compete with wild-type ZmKRPs 1, 2 or 5 to protect another corn-specific kinase complex, ZmCyclinD4/ZmCDKA;2.

FIG. 6 depicts protein sequences alignment of BnKRP1, AtKRP1, ZmKRP1, ZmKRP2, and ZmKRP5.

FIG. 7A and FIG. 7B depict protein sequences alignment of BnKRP1, BnKRP3, BnKRP4, BnKRP5, BnKRP6, ZmKRP1, ZmKRP2, ZmKRP3, ZmKRP4, ZmKRP5, ZmKRP6, ZmKRP7, and ZmKRP8.

### DESCRIPTION OF THE TEXT FILE SUBMITTED ELECTRONICALLY

The contents of the text file submitted electronically are incorporated herein by reference in their entirety: A computer readable format copy of the Sequence Listing (filename: TARG-013-01US_ST25.txt, date recorded: Nov. 14, 2011, file size 114 kilobytes).

### DETAILED DESCRIPTION

All publications, patents and patent applications, including any drawings and appendices, and all nucleic acid sequences and polypeptide sequences identified by GenBank Accession numbers, herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

### DEFINITIONS

As used herein, the verb “comprise” as is used in this description and in the claims and its conjugations are used in its non-limiting sense to mean that items following the word are included, but items not specifically mentioned are not excluded.

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots. Examples of particular plants include but are not limited to corn, potatoes, roses, apple trees, sunflowers, wheat, rice, bananas, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g. broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g. garlic, leek, onion (dry bulb, green, and Welch), shallot, and other bulb vegetable crops), citrus fruits (e.g. grapefruit, lemon, lime, orange, tangerine, citrus hybrids, pummelo, and other citrus fruit crops), cucurbit vegetables (e.g. cucumber, citron melon, edible gourds, gherkin, muskmelons (including hybrids and/or cultivars of cucumis melons), water-melon, cantaloupe, and other cucurbit vegetable crops), fruiting vegetables (including eggplant, ground cherry, pepino, pepper, tomato, tomatillo, and other fruiting vegetable crops), grape, leafy vegetables (e.g. romaine), root/tuber and corm vegetables (e.g. potato), and tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, lackberries, cloudberries, loganberries, raspberries, salmonberries, thimbleberries, and wineberries), cereal crops (e.g., corn, rice, wheat, barley, sorghum, millets, oats, ryes, triticales, buckwheats, fonio, quinoa, oil palm), pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fiber crops (e.g. hemp, cotton), ornamentals, and the like.

As used herein, the term “plant part” refers to any part of a plant including but not limited to the shoot, root, stem, seeds, stipules, leaves, petals, flowers, ovules, bracts, branches, petioles, internodes, bark, pubescence, tillers, rhizomes, fronds, blades, pollen, stamen, scion, rootstock, and the like. The two main parts of plants grown in some sort of media, such as soil, are often referred to as the “above-ground” part, also often referred to as the “shoots”, and the “below-ground” part, also often referred to as the “roots”.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “chimeric protein” or “recombinant protein” refers to a construct that links at least two heterologous proteins into a single macromolecule (fusion protein).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the terms “polypeptide,” “peptide,” and “protein” are used interchangeably herein to refer to polymers of amino acids of any length. These terms also include proteins that are post-translationally modified through reactions that include glycosylation, acetylation and phosphorylation.

As used herein, the term “homologous” or “homologue” or “ortholog” is known in the art and refers to related sequences that share a common ancestor or family member and are determined based on the degree of sequence identity. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “at least a portion” or “fragment” of a nucleic acid or polypeptide means a portion having the minimal size characteristics of such sequences, or any larger fragment of the full length molecule, up to and including the full length molecule. For example, a portion of a nucleic acid may be 12 nucleotides, 13 nucleotides, 14 nucleotides, 15 nucleotides, 16 nucleotides, 17 nucleotides, 18 nucleotides, 19 nucleotides, 20 nucleotides, 22 nucleotides, 24 nucleotides, 26 nucleotides, 28 nucleotides, 30 nucleotides, 32 nucleotides, 34 nucleotides, 36 nucleotides, 38 nucleotides, 40 nucleotides, 45 nucleotides, 50 nucleotides, 55 nucleotides, and so on, going up to the full length nucleic acid. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as hybridization probe may be as short as 12 nucleotides; in one embodiment, it is 20 nucleotides. A portion of a polypeptide useful as an epitope may be as short as 4 amino acids. A portion of a polypeptide that performs the function of the full-length polypeptide would generally be longer than 4 amino acids.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

As used herein, the term “substantially complementary” means that two nucleic acid sequences have at least about 65%, preferably about 70% or 75%, more preferably about 80% or 85%, even more preferably 90% or 95%, and most preferably about 98% or 99%, sequence complementarities to each other. This means that primers and probes must exhibit sufficient complementarity to their template and target nucleic acid, respectively, to hybridize under stringent conditions. Therefore, the primer and probe sequences need not reflect the exact complementary sequence of the binding region on the template and degenerate primers can be used. For example, a non-complementary nucleotide fragment may be attached to the 5′-end of the primer, with the remainder of the primer sequence being complementary to the strand. Alternatively, non-complementary bases or longer sequences can be interspersed into the primer, provided that the primer has sufficient complementarity with the sequence of one of the strands to be amplified to hybridize therewith, and to thereby form a duplex structure which can be extended by polymerizing means. The non-complementary nucleotide sequences of the primers may include restriction enzyme sites. Appending a restriction enzyme site to the end(s) of the target sequence would be particularly helpful for cloning of the target sequence. A substantially complementary primer sequence is one that has sufficient sequence complementarity to the amplification template to result in primer binding and second-strand synthesis. The skilled person is familiar with the requirements of primers to have sufficient sequence complementarity to the amplification template.

As used herein, the terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the phrase “a biologically active variant” or “functional variant” with respect to a protein refers to an amino acid sequence that is altered by one or more amino acids with respect to a reference sequence, while still maintains substantial biological activity of the reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

The Tm is the temperature (under defined ionic strength and pH) at which 50% of a complementary target sequence hybridizes to a perfectly matched probe or primer. Typically, stringent conditions will be those in which the salt concentration is less than about 1.0 M Na⁺ ion, typically about 0.01 to 1.0 M Na+ ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001.

As used herein, “coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence.

As used herein, “regulatory sequences” may include, but are not limited to, promoters, translation leader sequences, introns, and polyadenylation recognition sequences.

As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, a “plant promoter” is a promoter capable of initiating transcription in plant cells whether or not its origin is a plant cell, e.g. it is well known that Agrobactenum promoters are functional in plant cells. Thus, plant promoters include promoter DNA obtained from plants, plant viruses and bacteria such as Agrobacterium and Bradyrhizobium bacteria. A plant promoter can be a constitutive promoter or a non-constitutive promoter.

As used herein, a “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. Non-limiting exemplary constitutive promoters include, CaMV 35S promoter, opine promoters, ubiquitin promoter, actin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues, such as stems, leaves, roots, or seeds.

As used herein, “inducible” or “repressible” promoter is a promoter which is under chemical or environmental factors control. Examples of environmental conditions that may effect transcription by inducible promoters include anaerobic conditions, or certain chemicals, or the presence of light.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large amount of tissue-specific promoters isolated from particular plants and tissues found in both scientific and patent literature. Non-limiting tissue specific promoters include, beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression), and embryo specific promoters, e.g., a promoter associated with an amino acid permease gene (AAP1), an oleate 12-hydroxylase:desaturase gene from Lesquerella fendleri (LFAH12), an 2S2 albumin gene (2S2), a fatty acid elongase gene (FAE1), or a leafy cotyledon gene (LEC2).

As used herein, a “tissue preferred” promoter is a promoter that initiates transcription mostly, but not necessarily entirely or solely in certain tissues.

As used herein, a “cell type specific” promoter is a promoter that primarily drives expression in certain cell types in one or more organs, for example, vascular cells in roots, leaves, stalk cells, and stem cells.

As used herein, a “cell type preferred” promoter is a promoter that primarily drives expression mostly, but not necessarily entirely or solely in certain cell types in one or more organs, for example, vascular cells in roots, leaves, stalk cells, and stem cells.

As used herein, the “3′ non-coding sequences” or “3′ untranslated regions” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

As used herein, “RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

As used herein, the term “recombinant” refers to an artificial combination of two otherwise separated segments of sequence, e.g., by chemical synthesis or by the manipulation of isolated segments of nucleic acids by genetic engineering techniques.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

The term “expression”, as used herein, refers to the production of a functional end-product e.g., an mRNA or a protein (precursor or mature).

As used herein, the phrase “plant selectable or screenable marker” refers to a genetic marker functional in a plant cell. A selectable marker allows cells containing and expressing that marker to grow under conditions unfavorable to growth of cells not expressing that marker. A screenable marker facilitates identification of cells which express that marker.

As used herein, the term “inbred”, “inbred plant” is used in the context of the present invention. This also includes any single gene conversions of that inbred. The term single allele converted plant as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein, the term “sample” includes a sample from a plant, a plant part, a plant cell, or from a transmission vector, or a soil, water or air sample.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the terms “dicotyledon” and “dicot” refer to a flowering plant having an embryo containing two seed halves or cotyledons. Dicotyledon plants at least include the Eudicot, Magnoliid, Amborella, Nymphaeales, Austrobaileyales, Chloranthales, and Ceratophyllum groups. Eudicots include these clades: Ranunculales, sabiales, Proteales, Trochodendrales, Buxales, and Core Eudicots (e.g., Berberidopsidales, Dilleniales, Gunnerales, Caryophyllales, Santalales, Saxifragales, Vitales, Rosids and Asterids). Non-limiting examples of dicotyledon plants include tobacco, tomato, pea, alfalfa, clover, bean, soybean, peanut, members of the Brassicaceae family (e.g., camelina, Canola, oilseed rape, etc.), amaranth, sunflower, sugarbeet, cotton, oaks, maples, roses, mints, squashes, daisies, nuts; cacti, violets and buttercups.

As used herein, the term “monocotyledon” or “monocot” refer to any of a subclass (Monocotyledoneae) of flowering plants having an embryo containing only one seed leaf and usually having parallel-veined leaves, flower parts in multiples of three, and no secondary growth in stems and roots. Non-limiting examples of monocotyledon plants include lilies, orchids, corn, rice, wheat, barley, sorghum, millets, oats, ryes, triticales, buckwheats, fonio, quinoa, grasses, such as tall fescue, goat grass, and Kentucky bluegrass; grains, such as wheat, oats and barley, irises, onions, palms.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, organism (e.g., a plant), or group of organisms.

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the terms “mutant” or “mutation” refer to a gene, cell, or organism with an abnormal genetic constitution that may result in a variant phenotype. As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein when discussing plants, the term “ovule” refers to the female gametophyte, whereas the term “pollen” means the male gametophyte.

As used herein, the term “phenotype” refers to the observable characters of an individual cell, cell culture, organism (e.g., a plant), or group of organisms which results from the interaction between that individual's genetic makeup (i.e., genotype) and the environment.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “self-crossing”, “self pollinated” or “self-pollination” means the pollen of one flower on one plant is applied (artificially or naturally) to the ovule (stigma) of the same or a different flower on the same plant.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation. The original transformant is designated as “T0” or “T₀.” Selfing the T0 produces a first transformed generation designated as “T1” or “T₁.”

As used herein, the term “transgene” refers to a nucleic acid that is inserted into an organism, host cell or vector in a manner that ensures its function.

As used herein, the term “transgenic” refers to cells, cell cultures, organisms (e.g., plants), and progeny which have received a foreign or modified gene by one of the various methods of transformation, wherein the foreign or modified gene is from the same or different species than the species of the organism receiving the foreign or modified gene.

As used herein, the term “transposition event” refers to the movement of a transposon from a donor site to a target site.

As used herein, the term “variety” refers to a subdivision of a species, consisting of a group of individuals within the species that are distinct in form or function from other similar arrays of individuals.

As used herein, the term “vector”, “plasmid”, or “construct” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746).

As used herein, the phrase “seed size” refers to the volume of the seed material itself, which is the space occupied by the constituents of the seed.

As used herein, the phrase “seed number” refers to the average number of seeds produced from each fruit, each plant, or each predetermined area (e.g., 1 acre).

As used herein, the term “cyclin dependent kinase inhibitor” (also referred to herein as “CDK inhibitor” or “CKI”) refers to a class of proteins that negatively regulate cyclin dependent kinases (CDKs). CKIs amenable to the present invention are those having separate polypeptide regions capable of independently binding a cyclin and a CDK. Such CKIs include, for example, identified families of plant CKIs (the seven identified Arabidopsis CKIs), having homology to Kinase Inhibitor Proteins (KIPs) in animals, referred to as KIP-related proteins (KRPs) (also known as Inhibitors of “CDKs,” or “ICKs”).

The term “naturally occurring,” in the context of CKI polypeptides and nucleic acids, means a polypeptide or nucleic acid having an amino acid or nucleotide sequence that is found in nature, i.e., an amino acid or nucleotide sequence that can be isolated from a source in nature (an organism) and which has not been intentionally modified by human intervention. As used herein, laboratory strains of plants which may have been selectively bred according to classical genetics are considered naturally-occurring plants.

As used herein, “wild-type CKI gene” or “wild-type CKI nucleic acid” refers to a sequence of nucleic acid, corresponding to a CKI genetic locus in the genome of an organism, that encodes a gene product performing the normal function of the CKI protein encoded by a naturally-occurring nucleotide sequence corresponding to the genetic locus. A genetic locus can have more than one sequence or allele in a population of individuals, and the term “wild-type” encompasses all such naturally-occurring alleles that encode a gene product performing the normal function. “Wild-type” also encompasses gene sequences that are not necessarily naturally occurring, but that still encode a gene product with normal function (e.g., genes having silent mutations or encoding proteins with conservative substitutions).

As used herein, the term “wild-type CKI polypeptide” or “wild-type CKI protein” refers to a CKI polypeptide encoded by a wild-type gene. A genetic locus can have more than one sequence or allele in a population of individuals, and the term “wild-type” encompasses all such naturally-occurring alleles that encode a gene product performing the normal function.

As used herein, the phrase “dominant negative” in the context of protein mechanism of action or gene phenotype, refers to a mutant or variant protein, or the gene encoding the mutant or variant protein, that substantially or completely prevents a corresponding protein having wild-type function from performing the wild-type function. In the present invention, the ability of a mutant protein to prevent a corresponding protein having wild-type function can be evaluated in a kinase assay (the “in vitro KRP-Cyclin/CDK kinase assay”) as described herein, in which percent recovery of CDK kinase function is measured. A mutant KRP polypeptide is a dominant negative KRP if in said kinase assay the percent recovery of CDK kinase function with the presence of the mutant polypeptide and a corresponding wild-type KRP function is higher than the percent recovery of CDK kinase function with the presence of the corresponding protein wild-type KRP, but without the presence of the mutant KRP in said kinase assay. For example, the recovery of CDK kinase function with the presence of the mutant KRP and the corresponding wild-type KRP is at least 5%, at least 10%, at least 20%, at least 30%, at least 40%, at least 50%, at least 60%, at least 70%, at least 80%, at least 90%, at least 100%, at least 120%, at least 150%, at least 200%, or more compared to the percent recovery of CDK kinase function with the presence of the corresponding wild-type KRP, but without the presence of the mutant KRP.

### Kinase Inhibitor Protein (KIP) Related Protein (KRP)

Plants have cyclin dependent kinases (CDK) that regulate the transitions between different phases of the cell cycle (Verkest et al., 2005, Switching the Cell Cycle. Kip-Related Proteins in Plant Cell Cycle Control, Plant Physiology, November 2005, Vol. 139, pp. 1099-1106, incorporated by reference in its entirety herein).

In Arabidopsis (Arabidopsis thaliana), at least two classes of CDKs are involved in cell cycle regulation: the A-type CDKs that are represented by only one gene in the model species Arabidopsis (designated Arath;CDKA;1) and the B-type CDK family that has four members, grouped into the B1 (Arath;CDKB1;1 and Arath;CDKB1;2) and B2 (Arath; CDKB2;1 and Arath;CDKB2;2) subclasses (Vandepoele et al., 2002, Genome-wide analysis of core cell cycle genes in Arabidopsis. Plant Cell 14: 903-916). A-type CDKs display kinase activity from late G1 phase until the end of mitosis, suggesting a role for this particular CDK at both the G1-to-S and G2-to-M transition points (Magyar et al., 1997; Porceddu et al., 2001; Sorrell et al., 2001). A central role for CDKA;1 in controlling cell number has been demonstrated using transgenic tobacco (Nicotiana tabacum) plants with reduced A-type CDK activity (Hemerly et al., 1995). The requirement for Arath;CKDA;1 at least for entry into mitosis has been demonstrated as well by cdka;1 null mutants that fail to progress through the second mitosis during male gametophytic development (Nowack et al., 2005). The group of B-type CDKs displays a peak of activity at the G2-to-M phase transition only (Magyar et al., 1997; Porceddu et al., 2001; Sorrell et al., 2001), suggesting that they play a role at the onset of, or progression through, mitosis. Correspondingly, cells of plants with reduced B-type CDK activity arrest in the G2 phase of the cell cycle (Porceddu et al., 2001; Boudolf et al., 2004).

CDK is regulated by cyclins. Plant cyclins are very complicated. There are at least 49 different cyclins in Arabidopsis, which were classified into seven subclasses (A, B, C, D, H, P, and T) (Vandepoele et al., 2002; Wang et al., 2004). CDK are also regulated by docking of small proteins, generally known as CDK inhibitors (CKIs). CKIs have been identified in many organisms, e.g., budding yeast (Saccharomyces cerevisiae), fission yeast (Schizosaccharomyces pombe), mammals, and plants, see, Mendenhall, 1998; Kwon T. K. et al. 1998; Vlach J. et al. 1997; Russo et al., 1996; Wang et al., 1997, 1998 and 2000; Lui et al., 2000; De Veylder et al., 2001; Jasinski et al., 2002a, 2002b; Coelho et al., 2005; Jasinski S. et al., 2002, each of which is incorporated by reference in its entirety).

Plant CKIs are also known as KIP Related Proteins (KRPs). They have cyclin binding and CDK binding domains at their C-terminal, however the mechanism regulating this protein stability and function remains unknown (Zhou et al., 2003a; Weinl et al. 2005). KRP activity can be both regulated at the transcriptional level or at the posttranslational level (Wang et al., 1998; De Veylder et al., 2001; Jasinski et al., 2002b; Ormenese et al., 2004; Coqueret, 2003; Hengst, 2004; Verkest et al., 2005; Coelho et al., 2005, each of which is incorporated by reference in its entirety). KRPs in plant normally localize in nucleus (Jasinski et al., 2002b; Zhou et al., 2003a; Weinl et al., 2005).

KRP can function as an integrators of developmental signals, and control endocycle onset, in different cell cycle programs (e.g., proliferation, endoreduplication, and cell cycle exit). See Wang et al., 1998; Richard et al., 2001; Himanen et al., 2002; Grafi and Larkins, 1995; Joube's et al., 1999; Verkest et al., 2005; Weinl et al., 2005; Boudolf et al., 2004b.

### Mutant Maize KRP1 and KRP2 Proteins

The present invention is based on the discovery that KRP1 mutants derived from Brassica napus described in WO2007016319 (i.e., BnKRP1 DN#2 [F151A;F153A] (SEQ ID NO: 3) and BnKRP1 DN#3 [Y149A;F151A;F153A] (SEQ ID NO: 4) do not have dominant negative effect to prevent inhibition of maize Cyclin/CDK complex by maize KRP proteins, neither in vitro nor in vivo, even when they were codon optimized.

Without wishing to be bound by any theory, the inventors hypothesize that Brassica napus KRP mutant proteins do not act as dominant negatives against corn Cyclin/CDK complexes in corn plants. The inventors discovered that mutant Zea mays KRP1 (ZmKRP1) or KRP2 (ZmKRP2) can instead act as dominant negatives to prevent inhibition of corn Cyclin/CDK complexes by wild-type corn KRPs.

The present invention provides effective systems to test if a candidate mutant KRP protein can act as dominant negative mutant to prevent inhibition of a Cyclin/CDK complex by a wild-type KRP protein. The effective systems comprise a kinase assay (the “in vitro KRP-Cyclin-CDK kinase assay”), a non-limiting example of which is described herein.

In the assay, a candidate mutant KRP derived from a wild-type KRP of a plant species A, a wild-type cyclin protein of a plant species B, a wild-type CDK protein of the plant species B, and a wild-type KRP protein of the plant species B, are recombinantly expressed and purified. Then, the recombinant wild-type cyclin protein and the wild-type CDK protein are mixed to form a complex (alternatively, the cyclin protein and the CDK protein can be co-expressed and co-purified as a complex). In some embodiments, the recombinant proteins are expressed in insect cells. Plant species A can be the same as or different from plant species B. This kinase activity of said complex is then monitored with a standard kinase assay described below. This assay is referred as “in vitro KRP-Cyclin-CDK kinase assay” or simply as “kinase assay”. A substrate protein that can be activated (i.e., phosphorylated) by the Cyclin/CDK complex is selected, wherein such substrate protein can be, for example, Histone HI (HHI) or recombinant tobacco retinoblastoma protein (Nt Rb). Four mixtures can be made by adding recombinant proteins into a kinase buffer cocktail according to the table below:

TABLE 1
Components for Mixtures in Kinase Assay

| Compositions | Mixture I | Mixture II | Mixture III | Mixture IV |
|---|---|---|---|---|
| I. Kinase complex | at concentration | at concentration | at concentration | at concentration |
| comprising the wild-type | of C1 | of C1 | of C1 | of C1 |
| cyclin protein and the wild- | | | | |
| type CDK protein of the | | | | |
| plant species B | | | | |
| II. Wild-type KRP protein | 0 | at concentration | at concentration | 0 |
| of the plant species B | | of C2* | of C2 | |
| III. Candidate mutant KRP | 0 | 0 | at concentration | at concentration |
| derived from the wild-type | | | of C3** | of C3** |
| KRP of the plant species A | | | | |
| IV. Substrate | at concentration | at concentration | at concentration | at concentration |
| | of C4 | of C4 | of C4 | of C4 |
| Kinase Activity | 100% | X % | Y % | W % |
| | (no inhibition) | (wt inhibition) | (competition) | (mutant inhibition) |

*C2 is an amount of WT KRP that is sufficient to give between 0% and 20% kinase activity compared to mixture I.
**C3 should be no more than 50X C2

A non-limiting example of the kinase buffer cocktail comprises KAB: 50 mM Tris pH 8.0, 10 mM MgCl₂, 100 μM ATP plus 0.5 μCi/ml 32 PγATP and the substrate protein. Concentrations C1, C2, and C3 can be determined and optimized by one skilled in the art depending on experiment conditions.

To determine if a candidate mutant KRP is a DN mutant for the kinase complex, C2 should be about equimolar with C1; and, C3 should be no more than 50× of C2, or no more than 40× of C2, or no more than 30× of C2, or no more than 20× of C2, or no more than 10× of C2, or no more than 5× of C2. For example, in some instances the amount of C3 is about 1×, or about 2×, or about 3×, or about 4×, or about 5×, or about 6×, or about 7×, or about 8×, or about 9×, or about 10×, or about 11×, or about 12×, or about 13×, or about 14×, or about 15×, or about 16×, or about 17×, or about 18×, or about 19×, or about 20× of the amount of C2. In some situations, however, the amount of C3 may be about 25×, or about 30×, or about 35×, or about 40×, or about 45×, or about 50× of the amount of C2. As discussed elsewhere herein, the amount of C3 which is utilized in any particular situation must be physiologically achievable in a maize cell, tissue or whole plant in order to have a dominant negative effect on the wild-type KRP.

Composition I and/or Composition III are incubated on ice for a certain amount of time (e.g., 30 minutes). Subsequently, Composition II is then added to the mixture and incubated at 4° C. for certain amount of time (e.g., 30 mins) to allow binding to the kinase complex. The kinase reaction is then initiated by adding the buffer cocktail (KAB) and to the kinase complex mixture (I, II, III or IV) and incubated at 27° C. for a certain amount of time (e.g., 30 minutes) to allow reaction to complete. The kinase reaction in each mixture is stopped with an equal volume of 2× Laemmli buffer and boiled for 5 minutes. Next, monitor [³²P] phosphate incorporation to the substrate protein by autoradiography and/or Molecular Dynamics PhosphorImager following SDS-PAGE in each mixture. The signal strength of [³²P] phosphate incorporation in Mixture I is set as 100% percent recovery of kinase function. The strength of [³²P] phosphate incorporation in Mixture II is compared to that of Mixture I, calculated as X %; the strength of [³²P] phosphate incorporation in Mixture III is compared to that of Mixture I, calculated as Y %, the strength of [³²P] phosphate incorporation in Mixture IV is compared to that of Mixture I, calculated as W %. For example, if the signal strength is half of what is observed for Mixture I, the calculated percent recovery of kinase activity is 50%.

The X % is compared with Y %, and the dominant negative effect of the tested mutant KRP is calculated as follows: let Z %=(Y %−X %), and Zmax % is the maximum Z % within the allowable range of C2 and C3; if Zmax % is not statistically higher than 0% (i.e., Y %≦X %), the tested mutant KRP is not dominant negative against the tested wild-type KRP; if Zmax % is statistically higher that 0% (i.e., Y %>X %), but less than 30%, the tested mutant KRP is weakly dominant negative against the tested wild-type KRP; if Zmax % is higher that 30%, but less than 50%, the tested mutant KRP is substantially dominant negative against the tested wild-type KRP; if Zmax % is higher that 50%, the tested mutant KRP is strongly dominant negative against the tested wild-type KRP. A mutant KRP polypeptide is regarded as a dominant negative KRP which does not substantially inhibit kinase activity of the Cyclin/CDK complex, if W % is at least about 70%, for example, W % is at least about 70%, at least about 75%, at least about 80%, at least about 85%, at least about 90%, at least about 95%, at least about at 99%, or higher. In another words, the dominant negative KRPs of the present invention do not substantially inhibit the kinase activity of the cyclin/CDK complex, even when present in large molar excess over the cyclin/CDK complex. In some embodiments, a mutant KRP polypeptide is regarded as a dominant negative KRP which does not inhibit kinase activity of the Cyclin/CDK complex, if W % is at least about 90%, for example, the W % is at least about 91%, about 92%, about 93%, about 94%, about 95%, about 96%, about 97%, about 98%, at least about at 99%, or higher.

If the concentration of a candidate DN KRP is permitted to be arbitrarily high in in vitro assays, then many proteins might demonstrate DN-like activity in such assays. However, many or most of these candidate DN KRPs would be useless in vivo because they could not practically be expressed in sufficiently high amounts to achieve the desired DN KRP effect in maize cells, tissues and whole plants. Importantly, the present invention for the first time provides mutant KRPs that have a DN effect at expression levels that are physiologically achievable in maize.

Without wishing to be bound by any theory, a mutant KRP should have at least the following two features to be regarded as dominant negative KRP: (i) the mutant KRP polypeptide does not substantially inhibit kinase activity of the Cyclin/CDK complex; and (ii) the mutant KRP polypeptide can compete with one or more wild-type KRPs for binding to the CDK binding region. Whether a mutant KRP is a dominant negative KRP can be tested in the in vitro KRP-Cyclin-CDK kinase assay as defined herein. Therefore, as used herein, a mutant KRP is said to be able to protect a Zea mays Cyclin/CDK complex from a wild-type Zea mays KRP, or regarded as a dominant negative KRP, if in the in vitro KRP-Cyclin-CDK kinase assay as defined herein, the mutant KRP has a Z % value of at least higher than 0%. For example, the dominant negative mutant KRP has a Z % value of at least 5%, at least 10%, at least 20%, at least 30%, at least 40%, at least 50%, at least 60%, at least 70%, at least 80%, at least 90%, at least 99% or more. In some embodiments, the dominant negative KRP when expressed in a plant leads to increased seed number, seed size, and/or yield of the plant.

In some embodiments, the Cyclin/CDK complexes comprise a CDK protein selected from the group consisting of Zea mays CDK A;1 (ZmCDKA;1, SEQ ID NO: 53), Zea mays CDK A;2 (ZmCDKA;2, SEQ ID NO: 55), and a cyclin protein selected from the group consisting of Zea mays Cyclin D1, D2, D3, D4, D5, D6, D7, and combinations thereof, and the wild-type Zea mays KRPs are selected from the group consisting of ZmKRP1, ZmKRP2, ZmKRP3, ZmKRP4, ZmKRP5, ZmKRP6, ZmKRP7, ZmKRP8, and combinations thereof. For example, the wild-type Zea mays KRP is ZmKRP1, ZmKRP2, or ZmKRP5.

In some embodiments, the Zea mays cyclin is selected from the 59 cyclins described in Hu et al., 2010, which is incorporated herein by reference in its entirety. In some embodiments, Zea mays cyclin is selected from the 21 cyclin D proteins described in Hu et al., 2010. For example, the cyclin is selected from the group consisting of Zea mays cyclin D1;1, D2;1, D2;2, D3;1, D3;2, D4;1, D4;2, D4;3, D4;4, D4;5, D4;6, D4;7, D4;8, D4;9, D4;10, D5;1, D5;2, D5;3, D5;4, D6;1, D7;1, and combination thereof. In some embodiments, the cyclin is selected from the group consisting of SEQ ID NOs. 62 to 73, which are independently identified by the inventors of the present invention. It should be noted that the nomenclature in Hu et al. for certain cyclin proteins may or may not be the same as the nomenclature used for Zea mays cyclin proteins identified by the inventors.

In some embodiments, said mutant KRP is derived from Zea mays KRP1 (ZmKRP1; SEQ ID NO: 7) or KRP2 (ZmKRP2; SEQ ID NO: 11), with one or more mutations that cause the dominant negative phenotype. In some other embodiments, said mutant KRP1 or KRP2 is derived from a biologically active variant, or fragment thereof of wild-type ZmKRP1 or ZmKRP2. A candidate mutant KRP1 or KRP2 protein can be designed to add one or more modifications to the wild-type ZmKRP1 or ZmKRP2, or biologically active variant, or fragment thereof. Particularly suitable modifications include amino acid substitutions, insertions, or deletions. For example, amino acid substitutions can be generated as modifications in the CDK or the cyclin-binding region that reduce or eliminate binding. Similarly, amino acid substitutions can be generated as modifications in the CDK or the cyclin-binding region of the KRP that reduce or eliminate the inhibitory activity of the KRP towards the Cyclin/CDK complex. In typical embodiments, at least one non-conservative amino acid substitution, insertion, or deletion in the CDK binding region or the cyclin binding region is made to disrupt or modify binding of the CKI polypeptide to a CDK or cyclin protein. The substitutions may be single, where only one amino acid in the molecule has been substituted, or they may be multiple, where two or more amino acids have been substituted in the same molecule. Insertional zmKRP1 or ZmKRP2 mutants are those with one or more amino acids inserted immediately adjacent to an amino acid at a particular position in the wild-type ZmKRP1 or ZmKRP2 protein molecule, biologically active variant, or fragment thereof. The insertion can be one or more amino acids. The insertion can consist, e.g., of one or two conservative amino acids. Amino acids similar in charge and/or structure to the amino acids adjacent to the site of insertion are defined as conservative. Alternatively, mutant ZmKRP1 or ZmKRP2 protein includes the insertion of an amino acid with a charge and/or structure that is substantially different from the amino acids adjacent to the site of insertion.

Deletional ZmKRP1 or ZmKRP2 polypeptide mutants are those where one or more amino acids in the wild-type ZmKRP1 or ZmKRP2 protein molecule, biologically active variant, or fragment thereof, have been removed. In some embodiments, deletional mutants will have one, two or more amino acids deleted in a particular region of the ZmKRP1 or ZmKRP2. A candidate mutant ZmKRP1 or ZmKRP2 then can be tested in the kinase assay as described herein to decide if it is a dominant negative mutant ZmKRP1 or ZmKRP2.

General texts which describe molecular biological techniques, which are applicable to the present invention, such as cloning, mutation, cell culture and the like, include Berger and Kimmel, Guide to Molecular Cloning Techniques, Methods in Enzymology, Vol. 152 Academic Press, Inc., San Diego, Calif. (“Berger”); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating of KRP Thus, the invention also encompasses using known methods of protein engineering and recombinant DNA technology to improve or alter the characteristics of the KRP expressed in plants. Various types of mutagenesis can be used to produce and/or isolate variant nucleic acids that encode for protein molecules and/or to further modify/mutate the KRP. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

In one embodiment, biologically active variants of wild-type ZmKRP can be used. In some embodiments, the ZmKRP is ZmKRP1 or ZmKRP2. In some further embodiments, the biologically active variants share at least 47%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more amino acid sequence identity compared to ZmKRP1 or ZmKRP2. Manipulation of corresponding gene (including +/− upstream and downstream flanking regions) and ORF nucleotide sequences using standard procedures (e.g., site-directed mutagenesis or PCR) can be used to produce such variants. The simplest modifications involve the substitution of one or more amino acids for amino acids having similar biochemical properties. Said amino acid substitutions may be conservative or non-conservative.

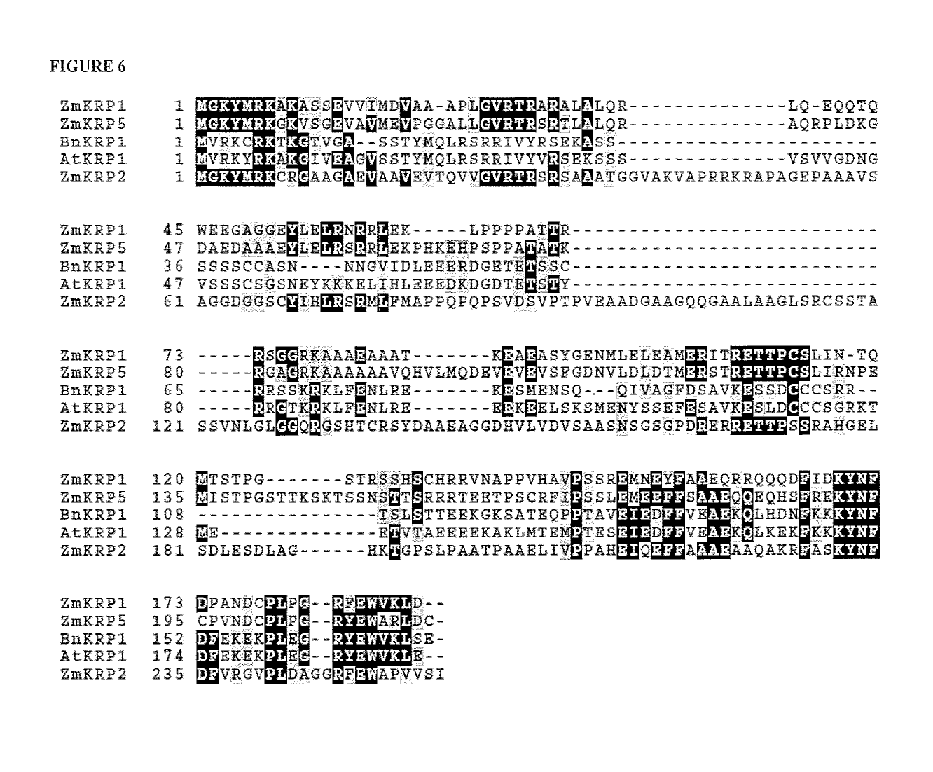

In some embodiments, a wild-type KRP protein from a species other than corn can be used to design candidate mutant KRP that can protect a corn Cyclin/CDK complex from a corn KRP. Without wishing to be bound by any theory, the reason why mutants BnKRP DN#2 and BnKRP DN#3 do not protect corn Cyclin/CDK complex may be due to their low identity to wild-type Zea mays KRPs. Sequence identity of BnKRP1, AtKRP1, ZmKRP1, ZmKRP2, and ZmKRP5 is shown in the table below, and alignment of these sequences is shown in FIG. 6.

TABLE 2
Identity between KRP Proteins

| | BnKRP1 | AtKRP1 | ZmKRP1 | ZmKRP2 | ZmKRP5 |
|---|---|---|---|---|---|
| BnKRP1 | 100% | 62% | 30% | 46% | 25% |
| AtKRP1 | 62% | 100% | 37% | 38% | 46% |
| ZmKRP1 | 30% | 37% | 100% | 26% | 51% |
| ZmKRP2 | 46% | 38% | 26% | 100% | 40% |
| ZmKRP5 | 25% | 46% | 51% | 40% | 100% |

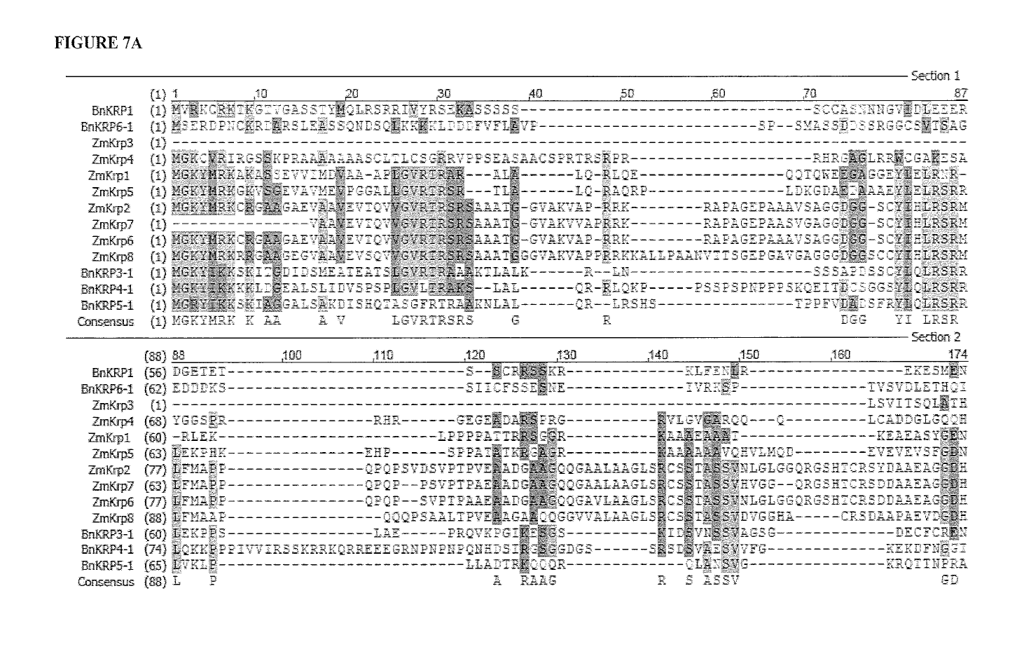

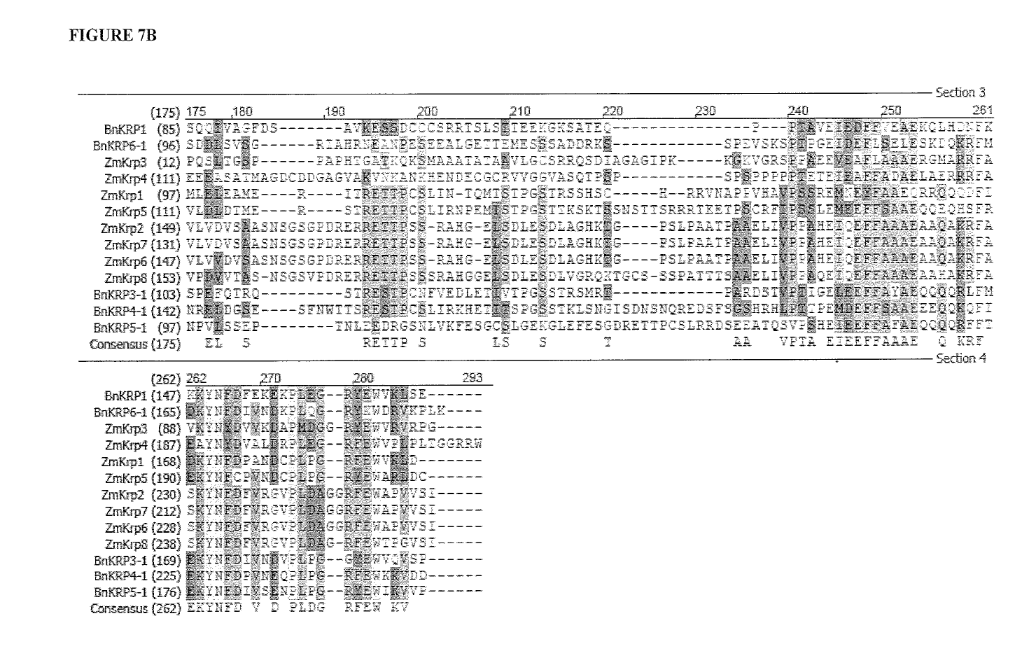

Sequence alignment of BnKRP1, BnKRP3, BnKRP4, BnKRP5, BnKRP6, ZmKRP1, ZmKRP2, ZmKRP3, ZmKRP4, ZmKRP5, ZmKRP6, ZmKRP7, and ZmKRP8 is shown in FIG. 7.

Therefore, a wild-type KRP protein from a species other than corn can be used to design candidate mutant KRP for maize, if said wild-type KRP from other species share at least 47%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more amino acid sequence identity to ZmKRP1 or ZmKRP2. For example, the wild-type ZmKRP1 or ZmKRP2 protein can be modified to create new ZmKRP1 or ZmKRP2 variants which substantially maintain the wild-type ZmKRP1 or ZmKRP2 activity, by introducing modifications inside or outside the conserved domains of the KRP protein. As used herein, the term “domain” generally refers to a portion of a protein or nucleic acid that is structurally and/or functionally distinct from another portion of the protein or nucleic acid. Amino acid substitutions outside of the conserved domains are less likely to affect protein function. Such a modified KRP protein can then be used to generate dominant negative KRPs by introducing mutations into the cyclin binding and/or CDK binding domain, for example, mutations at positions relative to amino acids 172 and 174 of ZmKRP1, or 234 and 236 of ZmKRP2.

Alternatively, variants of a dominant negative KRP of the present invention can be made. In some embodiments, amino acid substitutions are introduced in regions inside of the conserved domains of the KRP protein. For example, in some embodiments, modifications are introduced into a parent dominant negative KRP inside the cyclin binding and the CDK binding domains to create a new dominant negative KRP that is substantially bioactive as the parent mutant KRP. For example, the substitutions do not significantly reduce the value Z % of the dominant negative KRP in the kinase assay described herein. In some other embodiments, amino acid substitutions are introduced in regions outside of the conserved domains of a dominant negative KRP, wherein such amino acid substitutions do not substantially interfere with the dominant negative function of the KRP.

In another embodiment, more substantial changes in a wild-type KRP function or protein features may be obtained by selecting amino acid substitutions that are less conservative than conservative substitutions. In one specific, non-limiting, embodiment, such changes include changing residues that differ more significantly in their effect on maintaining polypeptide backbone structure (e.g., sheet or helical conformation) near the substitution, charge or hydrophobicity of the molecule at the target site, or bulk of a specific side chain. The following specific, non-limiting, examples are generally expected to produce the greatest changes in protein properties: (a) a hydrophilic residue (e.g., seryl or threonyl) is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histidyl) is substituted for (or by) an electronegative residue (e.g., glutamyl or aspartyl); or (d) a residue having a bulky side chain (e.g., phenylalanine) is substituted for (or by) one lacking a side chain (e.g., glycine). Although such a modified KRP may be less biological active compared to its wild-type, it can be still used as backbone to create a candidate dominant negative mutant KRP by introducing mutations into the cyclin binding and/or CDK binding domain, for example, mutations at positions relative to amino acids 172 or 174 of ZmKRP1, or amino acids 234 and 236 of ZmKRP2. Such a candidate can be subjected to the kinase assay as described herein to decide if it can be used as a dominant negative KRP. Alternatively, more substantial changes may be obtained and introduced into a dominant negative KRP, by selecting amino acid substitutions that are less conservative than conservative substitutions, so long as such amino acid substitutions do not completely remove dominant negative function of the mutant KRP. For example, the substitutions do not reduce the value Z % of the dominant negative KRP significantly lower than 0 in the kinase assay described herein.

Variant KRP sequences may be produced by standard DNA mutagenesis techniques. In one specific, non-limiting, embodiment, M13 primer mutagenesis is performed. Details of these techniques are provided in Sambrook et al. (In Molecular Cloning: A Laboratory Manual, Cold Spring Harbor Laboratory Press, New York, 1989), Ch. 15. By the use of such techniques, variants may be created that differ from the isomerases sequences. DNA molecules and nucleotide sequences that are derivatives of those specifically disclosed herein, and which differ from those disclosed by the deletion, addition, or substitution of nucleotides while still encoding a protein having the biological activity of the prototype enzyme. The resulting product gene can be cloned as a DNA insert into a vector. In many, but not all, common embodiments, the vectors of the present invention are plasmids or bacmids.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows non-limiting exemplary conservative amino acid substitutions.

TABLE 3
Conservation Amino Acid Substitution

| | Very Highly - | | |
|---|---|---|---|
| Original | Conserved | Highly Conserved Substitutions | Conserved Substitutions |
| Residue | Substitutions | (from the Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionally similar to a wild-type KRP.

In one embodiment, variants may differ from a KRP sequences described herein by alteration of the coding region to fit the codon usage bias of the particular organism into which the molecule is to be introduced. In other embodiments, the coding region may be altered by taking advantage of the degeneracy of the genetic code to alter the coding sequence such that, while the nucleotide sequence is substantially altered, it nevertheless encodes a protein having an amino acid sequence substantially similar to the wild-type KRP. For example, because of the degeneracy of the genetic code, four nucleotide codon triplets (GCT, GCG, GCC and GCA) code for alanine. The coding sequence of any specific alanine residue within a KRP, therefore, could be changed to any of these alternative codons without affecting the amino acid composition or characteristics of the encoded protein. Based upon the degeneracy of the genetic code, variant DNA molecules may be derived from the nucleic acid sequences disclosed herein using standard DNA mutagenesis techniques, as described herein, or by synthesis of DNA sequences.

Based on the polynucleotide sequences of a KRP gene and polypeptide sequences of a KRP protein described in the invention, one skilled in the art will be able to design variant nucleic acid sequences encoding a protein having similar function of the KRP by virtue of the degeneracy of the genetic code based on teaching of the present invention. One skilled in the art will also be able to isolate variant nucleic acid sequences encoding a protein having similar function of the KRP from a species other than those mentioned herein based on teaching of the present invention. In one embodiment, homologous genes from other species can be cloned by the classical approach, wherein it involves the purification of the target protein, obtaining amino acid sequences from peptides generated by proteolytic digestion and reverse translation of the peptides. The derived DNA sequence, which is bound to be ambiguous due to the degeneracy of the genetic code, can then be employed for the construction of probes to screen a gene library. In one embodiment, PCR methods can be used to isolate fragments of homologous genes containing at least two blocks of conserved amino acids. The amino acid sequence of a conserved region is reverse translated and a mixture of oligonucleotides is synthesized representing all possible DNA sequences coding for that particular amino acid sequence. Two such degenerate primer mixtures derived from appropriately spaced conserved blocks are employed in a PCR reaction. The PCR products are then, usually after enrichment for the expected fragment length, cloned and sequenced. In one embodiment, a homologous KRP gene can be isolated based on hybridization of two nucleic acid molecules under stringent conditions. More detailed methods of cloning homologous genes based on a known gene is described in “Gene Cloning and DNA Analysis: An Introduction”, (Publisher: John Wiley and Sons, 2010, ISBN 1405181737, 9781405181730), and “Gene cloning: principles and applications” (Publisher: Nelson Thornes, 2006).

In some embodiments, the invention provides modified dominant negative KRP genes that may comprise, mutations containing alterations that produce silent substitutions, additions, or deletions, but do not alter the properties or activities of the encoded dominant negative KRP proteins or how the proteins are made. Nucleotide variants can be produced for a variety of reasons, e.g., to optimize codon expression for a particular host (e.g., change codons in microbes to those preferred by plant cells).

In one embodiment, the invention provides chimeric proteins, wherein the chimeric proteins comprise polypeptide of a mutant KRP, or comprise variants and/or fragments of a mutant KRP, which is fused to one or more other polypeptides. Polynucleotides that encode such chimeric proteins can be cloned into an expression vector that can be expressed in a plant cell. In one embodiment, the polynucleotide encoding the KRP is operably linked to one or more DNA encoding a signal peptide which targets the fusion polypeptide produced therefrom to an organelle of the plant, wherein the seed weight, seed size, seed number and/or yield of the plant are increased.

In one embodiment, the KRP is any biologically active chimeric KRP designed in silico using gene shuffling and/or directed molecular evolution, wherein the chimeric KRP has at least 47% identity to ZmKRP1 or ZmKRP2.

Gene shuffling (a.k.a. DNA shuffling, or sexual PCR), is a way to rapidly propagate beneficial mutations in a directed evolution experiment. Gene shuffling provides new ways to improve the functionality of genes, thus improving traits and creating higher-performing products. Non-limiting exemplary methods of using gene shuffling to produce chimeric genes are described in U.S. Pat. Nos. 6,521,453, 6,423,542, 6,479,652, 6,368,861, 6,500,639, and U.S. Patent Application Publication Nos. 20060141626, 20040191772, 20040053267, 20030104417, and 20080171668, each of which is herein incorporated by reference in its entirety.

Directed evolution (DE) has in recent years emerged as an effective technique for generating and selecting proteins with a variety of uses. The starting point is usually a library containing proteins that already possess the desired function to some extent, although randomly generated proteins have also been used. Through a series of iterative steps, or ‘generations’, during each of which the proteins are diversified and then screened, the protein library is ‘evolved’ towards better performance. Several evoluted proteins have been described previously (see, Cherry and Fidantsef, 2003; Sylvestre et al., 2006; Yun et al. 2006; Chautard et al., 2007; Joyce, 1994; and Piatesi et al., 2006). Non-limiting exemplary methods of directed molecular evolution are described in Jackson et al. (Directed Evolution of Enzymes, Comprehensive Natural Products II, 2010, Chapter 9.20, Pages 723-749), Rubin-Pitel et al. (Directed Evolution Tools in Bioproduct and Bioprocess Development Bioprocessing for Value-Added Products from Renewable Resources, 2007, Pages 49-72), Reetz (Directed evolution of selective enzymes and hybrid catalysts, Tetrahedron, Volume 58, Issue 32, 5 Aug. 2002, Pages 6595-6602), Datamonitor (Datamonitor reports, Directed molecular evolution: product life cycle management for biologics, 2006, Electronic books), Brakmann and Johnsson (Directed molecular evolution of proteins: or how to improve enzymes for biocatalysis, Publisher: Wiley-VCH, 2002, ISBN 3527304231, 9783527304233), Davies (Directed molecular evolution by gene conversion, Publisher University of Bath, 2001), Arnold and Georgiou (Directed enzyme evolution: screening and selection methods, Publisher: Humana Press, 2003, ISBN 158829286X, 9781588292865), and directed evolution library creation: methods and protocols, Publisher Humana Press, 1984, ISBN 1588292851, 9781588292858), each of which is incorporated by reference in its entirety.

Computer-assistant design of directed evolution can be utilized, following the non-limiting exemplary strategies and methods as described in Wedge et al. (In silico modeling of directed evolution: Implications for experimental design and stepwise evolution, Journal of Theoretical Biology, Volume 257, Issue 1, 7 Mar. 2009, Pages 131-141), Knowles (Closed-loop evolutionary multiobjective optimization, IEEE Computational Intelligence Magazine 4 (3), art. no. 5190940, pp. 77-91), Francois and Hakim (Design of genetic networks with specified functions by evolution in silico, PNAS Jan. 13, 2004 vol. 101 no. 2 580-585), Sole et al. (Synthetic protocell biology: from reproduction to computation, Phil. Trans. R. Soc. B. 2007 362 (1486) 1727-1739), and Marguet et al. (Biology by design: reduction and synthesis of cellular components and behavior, J R Soc Interface 2007 4 (15) 607-623), each of which is incorporated by reference in its entirety.

A dominant negative KRP in the present invention can protect a corn Cyclin/CDK complex from inhibition by a wild-type corn KRP. However, it should be understood that such a dominant negative KRP can also be used to protect a Cyclin/CDK complex from inhibition by a corresponding KRP, wherein the Cyclin/CDK complex and the corresponding KRP are from a species other than corn, so long as the sequence identity of such a corresponding KRP is high enough, for example, at least 47% to ZmKRP1 or ZmKRP2. Some non-limiting examples of KRP sequences from other species sharing at least 47% to ZmKRP2 are shown in Table 4 below:

TABLE 4
Identity of Proteins in other plant species compared to ZmKRP2

| | | Identity |
|---|---|---|
| SEQ ID NO. | Plant Species/Gene ID | to ZmKRP2 |
| 30 | Sorghum bicolor (sorghum)/Sb_04g034040 | 81% |
| 31 | Oryza sativa/OsI_09039 (OsKRP1) | 58% |

Therefore, the dominant negative KRPs in the present invention can be used to protect a monocot plant Cyclin/CDK complex from inhibition by a corresponding KRP of said monocot plant. In some embodiments, said monocot plant is a corn, a sorghum plant, or a rice plant. In some embodiments, the nucleic acid sequence encoding the dominant negative KRP when incorporated into a plant leads to increased seed number, seed size, and/or yield of the plant.

### Expression Vectors

The present invention provides expression vectors comprising a polynucleotide having a nucleic acid sequence encoding a dominant negative KRP. In some embodiments, the dominant negative KRP is a mutant Zea mays KRP (ZmKRP), for example, ZmKRP1, ZmKRP2, or biologically active variant, or fragment thereof. The mutant ZmKRP1 or ZmKRP2 can protect one or more Zea mays Cyclin/CDK complex from one or more wild-type Zea mays KRPs.

In some embodiments, the backbone of the expression vectors can be any expression vectors suitable for producing transgenic plant, which are well known in the art. In one embodiment, the expression vector is suitable for expressing transgene in monocot plants, e.g., in cereal crops, such as maize, rice, wheat, barley, sorghum, millets, oats, ryes, triticales, buckwheats, fonio, quinoa and oil palm et al. In another embodiment, the expression vector is suitable for expressing transgene in dicot plants, such as beans, soybeans, peanuts, nuts, members of the Brassicaceae family (Camelina, oilseed rape, Canola, etc.), amaranth, cotton, peas, tomatoes, sugarbeet, and sunflower.

In one embodiment, the expression vector is an Agrobacterium binary vector (see, Karimi et al., Plant Physiol 145: 1183-1191; Komari et al., Methods Mol Biol 343: 15-42; Bevan M W (1984) Nucleic Acids Res 12: 1811-1821; Becker (1992), Plant Mol Biol 20: 1195-1197; Datla et al., (1992), Gene 122: 383-384; Hajdukiewicz (1994) Plant Mol Biol 25:989-994; Xiang (1999), Plant Mol Biol 40: 711-717; Chen et al., (2003) Mol Breed 11: 287-293; Weigel et al., (2000) Plant Physiol 122: 1003-1013). In another embodiment, the expression vector is a co-integrated vector (also called hybrid Ti plasmids). More expression vectors and methods of using them can be found in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830. Each of the references mentioned herein is incorporated by reference in its entirety.

In some embodiments, the nucleic acid sequence encoding mutant KRP is operably linked to a nucleic acid sequence of a plant promoter. Generally speaking, a plant promoter of the present invention can be a constitutive promoter, a non-constitutive promoter, an inducible promoter, or any other promoters, so long as the expression of the mutant KRP driven by the plant promoter can lead to increased average seed weight, seed size, seed number and/or yield.

A constitutive promoter is a promoter that is capable of directly or indirectly activating the transcription of one or more DNA sequences or genes in all tissues of a transgenic plant. Typically, a constitutive promoter such as the 35 S promoter of CaMC (Odell, Nature 313:810-812, 1985) is used. Other examples of constitutive promoters useful in plants include the opine promoter (e.g., U.S. Pat. No. 5,955,646), actin promoter (e.g., rice actin promoter, McElroy et al, Plant Cell 2:163-171, 1990; U.S. Pat. Nos. 5,641,876, 5,684,239, 6,750,378, 6,642,438, 6,462,258, 6,919,495), HE histone promoter (e.g., maize histone promoter, Lepetit et al., Mol Gen. Genet. 231:276-285, 1992), ubiquitin promoter (e.g., maize ubiquitin promoter, U.S. Pat. Nos. 5,510,474, 5,614,399, 6,020,190, 6,054,574; rice ubiquitin promoter, U.S. Pat. No. 6,528,701; sugarcane ubiquitin promoter, U.S. Pat. Nos. 6,706,948, 6,686,513 and 6,638,766), synthetic promoter (e.g., U.S. Pat. Nos. 6,072,050, 6,555,673) and the like.

An inducible promoter is a promoter that is capable of directly or indirectly activating transcription of one or more DNA sequences or genes in response to an inducer. In the absence of an inducer the DNA sequences or genes will not be transcribed. The inducer can be a chemical agent such as a protein, metabolite, a growth regulator, herbicide or a phenolic compound or a physiological stress imposed directly by heat, cold, salt, or toxic elements or indirectly through the action of a pathogen or disease agent such as a virus. A plant cell containing an inducible promoter can be exposed to an inducer by externally applying the inducer to the cell or plant such as by spraying, watering, heating or similar methods. If it is desirable to activate the expression of the target gene to a particular time during plant development, the inducer can be so applied at that time. Non-limiting examples of inducible promoter include heat shock promoters, a cold inducible promoter, such as the cold inducible promoter from B. napus (White et al., Plant Physiol. 106, 1994), the alcohol dehydrogenase promoter which is induced by ethanol (Nagao et al., Surveys of plant Molecular and Cell Biology Vol. 3, p 384-438 (B. J. Miflin ed., Oxford University Press, Oxford, 1986; U.S. Pat. Nos. 5,001,060 and 5,290,924).

In one embodiment, the promoter is a tissue specific or tissue preferred promoter. In one embodiment, the tissue specific or tissue preferred promoters of the present invention useful for expressing dominant negative KRP in plant are embryo-specific promoter, an endosperm-specific promoter, or an ear-specific promoter. In some embodiments, the promoter is a development stage-specific promoter, for example, promoter sequences that initiate expression in embryo development, such as during early phase-specific embryo development. An early phase-specific promoter is a promoter that initiates expression of a protein prior to day 7 after pollination (walking stick) in Arabidopsis or an equivalent stage in another plant species. Non-limiting examples of promoters include a promoter for the amino acid permease gene (AAP1) (e.g., the AAP1 promoter from Arabidopsis thaliana) (Hirner et al., Plant J. 14:535-544, 1998), a promoter for the oleate 12-hydroxylase:desaturase gene (e.g., the promoter designated LFAH12 from Lesquerella fendleri) (Broun et al., Plant J. 13:201-210, 1998), a promoter for the 2S2 albumin gene (e.g., the 2S2 promoter from Arabidopsis thaliana) (Guerche et al., Plant Cell 2:469-478, 1990), a fatty acid elongase gene promoter (FAE1) (e.g., the FAE1 promoter from Arabidopsis thaliana) (Rossak et al., Plant Mol. Biol. 46:717-715, 2001), and the leafy cotyledon gene promoter (LEC) (e.g., the LEC2 gene promoter from Arabidopsis thaliana, see Kroj et al., Development 130:6065-6073, 2003, or corn LEC1 gene (ZmLEC1), see Zhang, et al., Planta, 215(2):191-194). Other early embryo-specific promoters of interest include, but are not limited to, ZmLEC1 (Zhang et al., Planta 215(2): 191-194), OsASP1 (Bi et al., Plant Cell Physiol 4691): 87-98), Seedstick (Pinyopich et al., Nature 424:85-88, 2003), Fbp7 and Fbp11 (Petunia Seedstick) (Colombo et al., Plant Cell. 9:703-715, 1997), Banyuls (Devic et al., Plant J. 19:387-398, 1999), ag1-15 and ag1-18 (Lehti-Shiu et al., Plant Mol. Biol. 58:89-107, 2005), Phel (Kohler et al., Genes Develop. 17:1540-1553, 2003), Pert (Haslekas et al., Plant Mol Biol. 36:833-845, 1998; Haslekas et al., Plant Mol. Biol. 53:313-326, 2003), emb175 (Cushing et al., Planta 221:424-436, 2005), L11 (Kwong et al., Plant Cell 15:5-18, 2003), Lec1 (Lotan et al., Cell 93:1195-1205, 1998), Fusca3 (Kroj et al., Development 130:6065-6073, 2003), tt12 (Debeaujon et al., Plant Cell 13:853-871, 2001), tt16 (Nesi et al., Plant Cell 14:2463-2479, 2002), A-RZf (Zou and Taylor, Gene 196:291-295, 1997), TtG1 (Walker et al., Plant Cell 11:1337-1350, 1999; Tsuchiya et al., Plant J. 37:73-81, 2004), Tt1 (Sagasser et al., Genes Dev. 16:138-149, 2002), TT8 (Nesi et al., Plant Cell 12:1863-1878, 2000), Gea-8 (carrot) (Lin and Zimmerman, J. Exp. Botany 50:1139-1147, 1999), Knox (rice) (Postma-Haarsma et al., Plant Mol. Biol. 39:257-271, 1999), Oleosin (Plant et al., Plant Mol. Biol. 25:193-205, 1994; Keddie et al., Plant Mol. Biol. 24:327-340, 1994; Qu et al., J Biol Chem. 1990 Feb. 5; 265(4):2238-2243), ABI3 (Ng et al., Plant Mol. Biol. 54:25-38, 2004; Parcy et al., Plant Cell 6:1567-1582, 1994), and the like. All references cited herein are incorporated by reference in their entireties for all purposes. Also, any promoter homologous to (or having a high sequence identity to) any of the promoters mentioned and also exhibiting stem specific expression, as defined herein, can be used. Other plant promoters that can be used include, but are not limited to, promoters associated with Period circadian protein (PER) genes, (e.g., Hordeum vulgare PER1 (HvPER1) gene, see Stacy et al., Plant Journal, 19(1):1-8, 1999), END genes, (anther-specific gene, e.g., END2 gene), zein genes (endosperm-specific genes, e.g., CZ19B1 gene, U.S. Pat. No. 6,225,529 and Reyes et al., Plant Physiology, 153:624-631), Early Extra Petals genes (e.g., EEP1 gene), Protein phosphatase genes (e.g., PP1A gene), ABI3 gene, Ubiquitin gene, an aspartic protease 1 gene (ASP1), a legumin 1A (LEG1A) gene, an AGAMOUS gene or a CLAVATA1 gene (CLV1).

For example, the AAP1 promoter is the AAP1 promoter from Arabidopsis thaliana (SEQ ID NO: 40), or functional part thereof, the oleate 12-hydroxylase:desaturase promoter is the oleate 12-hydroxylase:desaturase gene promoter from Lesquerella fendleri (LFAH12, SEQ ID NO: 41), or functional part thereof, the 2S2 gene promoter is from Arabidopsis thaliana, the fatty acid elongase gene promoter is from Arabidopsis thaliana, the leafy cotyledon gene promoter is from Arabidopsis thaliana, or functional part thereof, the oleosin gene promoter is from Zea mays (SEQ ID NO: 32), or functional part thereof, the leafy cotyledon 1 (LEC1) gene promoter is from Zea mays (ZmLEC1) (SEQ ID NO: 35), or functional part thereof, the aspartic protease 1 (ASP1) gene promoter is from Oryza sativa or Zea mays (OsAsp1; ZmAsp1), or functional part thereof, the legumin 1A (LEG1A) gene promoter is from Zea mays (ZmLEG1A, SEQ ID NO: 42), or functional part thereof, the AGAMOUS gene promoter is from Zea mays (ZmZAG1, SEQ ID NO: 43), or functional part thereof, or the CLAVATA 1 gene promoter is from Zea mays (ZmCLV1), or functional part thereof.

Other embryo-specific promoters of interest include the promoters from the following genes: Seedstick (Pinvopich et al., Nature 424:85-88, 2003), Fbp7 and Fbp11 (Petunia Seedstick) (Colombo et al., Plant Cell. 9:703-715, 1997), Banyuls (Devic, Plant J., 19:387-398, 1999), ABI3 (Ng et al., Plant. Mol. Biol. 54:25-38, 2004), ag1-15, Agl18 (Lehti-Shiu et al., Plant Mol. Biol. 58:89-107, 2005), Phel (Kohler, Genes Develop. 17:1540-1553, 2003), emb175 (Cushing et al., Planta. 221:424-436, 2005), L11 (Kwong et al., Plant Cell 15:5-18, 2003), Lec1 (Lotan, Cell 93:1195-1205, 1998), Fusca3 (Kroj et al., Development 130:6065-6073, 2003), TT12 (Debeaujon et al., Plant Cell 13:853-871, 2001), TT16 (Nesi et al., Plant Cell 14:2463-2479, 2002), A-RZf (Zou and Taylor, Gene 196:291-295, 1997), TTG1 (Walker et al., Plant Cell 11:1337-1350, 1999), TT1 (Sagasser et al., Genes Dev. 16:138-149, 2002), TT8 (Nesi et al., Plant Cell 12:1863-1878, 2000), and Gea-8 (carrot) (Lin et al., J. Exp. Botany 50:1139-1147, 1999) promoters. Embryo specific promoters from monocots include Globulin, Knox (rice) (Postma-Haarsma, Plant Mol. Biol. 39:257-271, 1999), Oleosin (Plant, Plant Mol. Biol. 25:193-205, 1994), Keddie, Plant Mol. Biol. 24:327-340, 1994), Peroxiredoxin (Per1) (Haslekas et al., Plant Mol. Biol. 36:833-845, 1998), Haslekas et al., Plant Mol. Biol. 53:313-326, 2003), HvGAMYB (Diaz et al., Plant J. 29:453-464, 2002) and SAD1 (Isabel-LaMoneda et al., Plant J. 33:329-340, 1999) from Barley, and Zea mays Hybrid proline rich protein promoters (Jose-Estanyol et al., Plant Cell 4:413-423, 1992; Jose-Estanyol et al., Gene 356:146-152, 2005).

In some embodiments, the promoter is a seed storage protein. Suitable seed storage protein promoters for dicotyledonous plants include, for example, bean β-phaseolin, lectin, and phytohemagglutinin promoters (Sengupta-Gopalan, et al., Proc. Natl. Acad. Sci. U.S.A. 82:3320-3324, 1985; Hoffman et al., Plant Mol. Biol. 11:717-729, 1988; Voelker et al., EMBO J. 6:3571-3577, 1987); rapeseed (Canola) napin promoter (Radke et al., Theor. Appl. Genet. 75:685-694, 1988); soybean glycinin and conglycinin promoters (Chen et al., EMBO J. 7:297-302, 1988; Nielson et al., Plant Cell 1:313-328, 1989, Harada et al., Plant Cell 1:415-425, 1989; Beachy et al., EMBO J. 4:3047-3053, 1985); soybean lectin promoter (Okamuro et al., Proc. Natl. Acad. Sci. USA 83:8240-8244, 1986); soybean Kunitz trypsin inhibitor promoter (Perez-Grau et al., Plant Cell 1:1095-1109, 1989; Jofuku et al., Plant Cell 1:1079-1093, 1989); potato patatin promoter (Rocha-Sosa et al., EMBO J. 8:23-29, 1989); pea convicilin, vicilin, and legumin promoters (Rerie et al., Mol. Gen. Genet. 259:148-157, 1991; Newbigin et al., Planta 180:461-470, 1990; Higgins et al., Plant Mol. Biol. 11:683-695, 1988; Shirsat et al., Mol. Gen. Genetics 215:326-331, 1989); and sweet potato sporamin promoter (Hattori et al., Plant Mol. Biol. 14:595-604, 1990). Non-limiting exemplary sequences of promoters associated with corn oleosin gene are described in WO/1999/064579; non-limiting exemplary sequences of promoters associated with corn legumin gene are described in US Patent Publication No. 20060130184; and non-limiting exemplary sequences of promoters associated with corn AGAMOUS (ZAG1) gene are described in Schmidt et al. (Plant Cell, 1993 July; 5(7):729-37), each of which is incorporated by reference in its entirety. For monocotyledonous plants, seed storage protein promoters useful in the practice of the invention include, e.g., maize zein promoters (Schernthaner et al., EMBO J. 7:1249-1255, 1988; Hoffman et al., EMBO J. 6:3213-3221, 1987 (maize 15 kD zein)); maize 18 kD oleosin promoter (Lee et al., Proc. Natl. Acad. Sci. USA 888:6181-6185, 1991); waxy promoter; shrunken-1 promoter; globulin 1 promoter; shrunken-2 promoter; rice glutelin promoter; barley hordein promoter (Marris et al., Plant Mol. Biol. 10:359-366, 1988); RP5 (Su et al., J. Plant Physiol. 158:247-254, 2001); EBEL and 2 maize promoters (Magnard et al., Plant Mol. Biol. 53:821-836, 2003) and wheat glutenin and gliadin promoters (U.S. Pat. No. 5,650,558; Colot et al., EMBO J. 6:3559-3564, 1987).

Optionally, the nucleic acid sequence encoding a dominant negative KRP is also operably linked to a plant 3′ non-translated region (3′ UTR). A plant 3′ non-translated sequence is not necessarily derived from a plant gene. For example, it can be a terminator sequence derived from viral or bacterium gene, or T-DNA. The 3′ non-translated regulatory DNA sequence can include from about 20 to 50, about 50 to 100, about 100 to 500, or about 500 to 1,000 nucleotide base pairs and may contain plant transcriptional and translational termination sequences in addition to a polyadenylation signal and any other regulatory signals capable of effecting mRNA processing or gene expression. Non-limiting examples of suitable 3′ non-translated sequences are the 3′ transcribed non-translated regions containing a polyadenylation signal from the nopaline synthase (NOS) gene of Agrobacterium tumefaciens (Bevan et al., 1983, Nucl. Acid Res., 11:369), or terminator for the T7 transcript from the octopine synthase gene of Agrobacterium tumefaciens. More suitable 3′ non-translated sequences include, 3′UTR of the potato cathepsin D inhibitor gene (GenBank Acc. No.: X74985), 3′UTR of the field bean storage protein gene VfLEIB3 (GenBank Acc. No.: Z26489), 3′UTR of pea E9 small subunit of the ribulose-1,5-bisphosphate carboxylase (ssRUBISCO) gene, 3′UTR of pea bcs, the tml terminator, the AHAS large and small subunit terminators, and OCS gene (octopene synthase) terminator. Each of the publications on plant 3′ non-translated region mentioned herein is incorporated by reference in its entirety. The plant 3′ non-translated regions and plant promoters mentioned herein can be used in vectors for both monocotyledon and dicotyledon transformations.

The expression vectors of the present invention further comprise nucleic acids encoding one or more selection markers. The selection marker can be a positive selectable marker, a negative selectable marker, or combination thereof. A “positive selectable marker gene” encodes a protein that allows growth on selective medium of cells that carry the marker gene, but not of cells that do not carry the marker gene. Selection is for cells that grow on the selective medium (showing acquisition of the marker) and is used to identify transformants. A common example is a drug-resistance marker such as NPT (neomycin phosphotransferase), whose gene product detoxifies kanamycin by phosphorylation and thus allows growth on media containing the drug. Other positive selectable marker genes for use in connection with the present invention include, but are not limited to, a Neo gene (Potrykus et al., 1985), which codes for kanamycin resistance and can be selected for using kanamycin, G418, etc.; a bar gene from Streptomyces hygroscopicus, which codes for a phosphinothricin acetyl transferase giving bialaphos (basta) resistance; a mutant aroA gene, which encodes an altered EPSP synthase protein (Hinchee et al., 1988), thus conferring glyphosate resistance; a nitrilase gene such as bxn from Klebsiella ozaenae, which confers resistance to bromoxynil (Stalker et al., 1988); a mutant acetolactate synthase gene (ALS), which confers resistance to imidazolinone, sulfonylurea or other ALS inhibiting chemicals (European Patent Application 154,204,1985); a methotrexate resistant DHFR gene (Thillet et al., 1988), or a dalapon dehalogenase gene that confers resistance to the herbicide dalapon; the pat gene from Streptomyces viridochromogenes, which encodes the enzyme phosphinothricin acetyl transferase (PAT) and inactivates the active ingredient in the herbicide bialaphos, phosphinothricin (PPT); or a mutated anthranilate synthase gene that confers resistance to 5-methyl tryptophan. Additional positive selectable marker genes include those genes that provide resistance to environmental factors such as excess moisture, chilling, freezing, high temperature, salt, and oxidative stress. Of course, when it is desired to introduce such a trait into a plant as a “gene of interest”, the selectable marker cannot be one that provides for resistance to an environmental factor.

Markers useful in the practice of the claimed invention include: an “antifreeze” protein such as that of the winter flounder (Cutler et al., 1989) or synthetic gene derivatives thereof genes which provide improved chilling tolerance, such as that conferred through increased expression of glycerol-3-phosphate acetyltransferase in chloroplasts (Murata et al., 1992; Wolter et al., 1992); resistance to oxidative stress conferred by expression of superoxide dismutase (Gupta et al., 1993), and may be improved by glutathione reductase (Bowler et al., 1992); genes providing “drought resistance” and “drought tolerance”, such as genes encoding for mannitol dehydrogenase (Lee and Saier, 1982) and trehalose-6-phosphate synthase (Kaasen et al., 1992).

A “negative selectable marker gene” encodes a protein that prevents the growth of a plant or plant cell on selective medium of plants that carry the marker gene, but not of plants that do not carry the marker gene. Selection of plants that grow on the selective medium provides for the identification of plants that have eliminated or evicted the selectable marker genes. An example is CodA (Escherichia coli cytosine deaminase), whose gene product deaminates 5-fluorocytosine (which is normally non-toxic as plants do not metabolize cytosine) to the toxic 5-fluorouracil. Other negative selectable markers include the haloalkane dehalogenase (dhlA) gene of Xanthobacter autotrophicus GJ10 which encodes a dehalogenase, which hydrolyzes dihaloalkanes, such as 1,2-dichloroethane (DCE), to a halogenated alcohol and an inorganic halide (Naested et al., 1999, Plant J. 18 (5): 571-6). Each of the publications on selectable markers mentioned herein is incorporated by reference in its entirety.

Optionally, additional nucleic acid sequence can be included into the expression vectors of the present invention to facilitate the transcription, translation, and post-translational modification, so that expression and accumulation of active dominant negative KRP in a plant cell are increased. Such additional nucleic acid sequence can enhance either the expression, or the stability of the protein. In one embodiment, such nucleic acid is an intron that has positive effect on gene expression, which has been also known as intron-mediated enhancement (IME, see Mascarenhas et al., (1990). Plant Mol. Biol. 15: 913-920). IME has been observed in a wide range of eukaryotes, including vertebrates, invertebrates, fungi, and plants (see references 17-26), suggesting that it reflects a fundamental feature of gene expression. In many cases, introns have a larger influence than do promoters in determining the level and pattern of expression. Non-limiting IME in plants have been described in Rose et al. (The Plant Cell 20:543-551 (2008)); Lee et al. (Plant Physiology 145:1294-1300 (2007)); Casas-Mollano et al. (Journal of Experimental Botany Volume 57, Number 12 Pp. 3301-3311); Jeong et al. (Plant Physiology 140:196-209 (2006)); Clancy et al. (Plant Physiol, October 2002, Vol. 130, pp. 918-929); Jeon et al. (Plant Physiol, July 2000, Vol. 123, pp. 1005-1014); Rose et al. (Plant Physiol, February 2000, Vol. 122, pp. 535-542); Kim et al. (Plant Physiol. (1999) 121: 225-236), and Callis et al. (Genes Dev. 1987 1: 1183-1200). Each of the publications on IMEs mentioned herein is incorporated by reference in its entirety. Thus, in one embodiment, any one of the IME described herein can be included in the expression vectors of the present invention. For example, the first intron (SEQ ID NO: 44) of ADH1 (Alcohol Dehydrogenase 1) gene can be included upstream of the initiator methionine to increase expression (see Callis et al., Genes Dev. 1987 1: 1183-1200).

The expression vectors of the present invention can be transformed into a plant to increase the seed weight, seed size, seed number and/or yield thereof, using the transformation methods described separately below. Thus, the present invention provides transgenic plants transformed with the expression vectors as described herein. The plant can be any plant in which an increased seed weight, seed size, seed number and/or yield is preferred by breeders for any reasons, e.g., for economical/agricultural interests. In one embodiment, said plants are dicotyledon plants. For example, the plant is a bean plant, a soybean plant, peanuts, nuts, members of the Brassicaceae family (Camelina, oilseed rape, Canola, etc.), amaranth, cotton, peas, tomatoes, sugarbeet, sunflower. In another embodiment, said plants are monocotyledon plants. For example, the plant is corn, rice, wheat, barley, sorghum, millets, oats, ryes, triticales, buckwheats, fonio, quinoa, oil palm.

Methods of Increasing Seed Weight, Seed Size, Seed Number and/or Seed Yield

The present invention provides methods of increasing seed weight, seed size, seed number and/or seed yield. In one embodiment, the methods comprise incorporating the dominant negative KRP of the present invention as described herein into a plant. One skilled in the art would be able to select suitable methods of incorporation.

The dominant negative KRP can be incorporated into a plant by transforming the plant with an expression vector of the present invention as described elsewhere herein. The dominant negative KRP can also be incorporated into a plant by breeding methods. For example, a transgenic plant comprising the dominant negative KRP of the present invention can be crossed to a second plant to produce a progeny wherein new transgenic plants comprising the dominant negative KRP can be isolated. Methods of breeding are discussed separately below.

Any transgenic plant with increased seed weight, seed size, seed number and/or yield generated from the present invention comprising a dominant negative KRP can be used as a donor to produce more transgenic plants through plant breeding methods well known to those skilled in the art. The goal in general is to develop new, unique and superior varieties and hybrids. In some embodiments, selection methods, e.g., molecular marker assisted selection, can be combined with breeding methods to accelerate the process.

In one embodiment, said method comprises (i) crossing any one of the plants of the present invention comprising a dominant negative KRP with increased seed weight, seed size, seed number and/or yield as a donor to a recipient plant line to create a F1 population; (ii) evaluating seed weight, seed size, seed number and/or yield in the offsprings derived from said F1 population; and (iii) selecting offsprings that have increased seed weight, seed size, seed number and/or yield.

In one embodiment, complete chromosomes of the donor plant are transferred. For example, the transgenic plant with increased seed weight, seed size, seed number and/or yield can serve as a male or female parent in a cross pollination to produce offspring plants, wherein by receiving the transgene from the donor plant, the offspring plants have increased seed weight, seed size, seed number and/or yield.

In a method for producing plants having increased seed weight, seed size, seed number and/or yield, protoplast fusion can also be used for the transfer of the transgene from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, that may even be obtained with plant species that cannot be interbred in nature, is tissue cultured into a hybrid plant exhibiting the desirable combination of traits. More specifically, a first protoplast can be obtained from a plant having increased seed weight, seed size, seed number and/or yield. A second protoplast can be obtained from a second plant line, optionally from another plant species or variety, preferably from the same plant species or variety, that comprises commercially desirable characteristics, such as, but not limited to disease resistance, insect resistance, valuable grain characteristics (e.g., increased seed weight, seed size, seed number and/or yield) etc. The protoplasts are then fused using traditional protoplast fusion procedures, which are known in the art to produce the cross.

Alternatively, embryo rescue may be employed in the transfer of dominant negative KRP from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (see Pierik, 1999, in vitro culture of higher plants, Springer, ISBN 079235267x, 9780792352679, which is incorporated herein by reference in its entirety).

In one embodiment, the recipient plant is an elite line having one or more certain agronomically important traits. As used herein, “agronomically important traits” include any phenotype in a plant or plant part that is useful or advantageous for human use. Examples of agronomically important traits include but are not limited to those that result in increased biomass production, production of specific biofuels, increased food production, improved food quality, etc. Additional examples of agronomically important traits includes pest resistance, vigor, development time (time to harvest), enhanced nutrient content, novel growth patterns, flavors or colors, salt, heat, drought and cold tolerance, and the like. Agronomically important traits do not include selectable marker genes (e.g., genes encoding herbicide or antibiotic resistance used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, β-glucuronidase, chloramphenicol acetyl transferase (CAT, etc.). For example, the recipient plant can be a plant with increased seed weight, seed size, seed number and/or yield which is due to a trait related to other dominant negative KRP, or a trait not related to dominant negative KRP, such as traits in the plants created by the REVOLUTA protein related techniques described in WO 2007/016319 and WO 2007/079353, which are incorporated herein by reference in their entireties. The recipient plant can also be a plant with preferred carbohydrate composition, e.g., composition preferred for nutritional or industrial applications, especially those plants in which the preferred composition is present in seeds.

Transgenic Plants with Increased Average Seed Weight, Seed Size, Seed Number and/or Yield

The present invention provides transgenic plants expressing a dominant negative KRP, biologically active variants, or fragments thereof, wherein the dominant negative KRP, biologically active variants, or fragments thereof can protect a corn Cyclin/CDK complex from inhibition by a corn wild-type KRP, and wherein transgenic plant has increased seed weight and/or seed size compared to a control plant not expressing the dominant negative KRP, biologically active variants, or fragments thereof.

In some embodiments, the mutant KRP comprises amino acid sequence having at least one modification relative to a wild-type KRP, biologically active variant, or fragment thereof, said wild-type KRP polypeptide comprises (a) a cyclin binding region conferring binding affinity for a cyclin and (b) a cyclin-dependent kinase (CDK) binding region conferring binding affinity for a CDK, wherein the wild-type KRP has at least 47% identity to Zea mays KRP1 (ZmKRP1) or KRP2 (ZmKRP2); wherein the mutant KRP polypeptide does not inhibit kinase activity of the Cyclin/CDK complex; and wherein the mutant KRP polypeptide can compete with one or more wild-type Zea mays KRPs for binding to the CDK binding region. In some embodiments, the wild-type KRP is ZmKRP2. In some further embodiments, the mutant KRP comprises at least two modifications relative to ZmKRP2 (SEQ ID NO: 11) at amino acid position 234 and position 236, for example, the mutant KRP comprises SEQ ID NO: 12. In some embodiments, the wild-type KRP is ZmKRP1. In some further embodiments, the mutant KRP comprises at least two modifications relative to ZmKRP1 (SEQ ID NO: 4) at amino acid position 172 and position 174, for example, the mutant KRP comprises SEQ ID NO: 8.

In one embodiment, said plant is a dicotyledonous plant, or dicotyledon or dicot. In another embodiment, said plant is a monocotyledonous plant, or monocotyledon or monocot. The plant can be any plant wherein an increased seed weight, seed size, seed number and/or yield are of interest. For example, the plant is a dicotyledon plant, e.g., bean, soybean, peanut, nuts, members of the Brassicaceae family (Camelina, oilseed rape, Canola, etc.), amaranth, cotton, peas, sunflower, or a monocotyledon plant, such as a cereal crop, e.g., corn, rice, wheat, barley, sorghum, millets, oats, ryes, triticales, buckwheats, fonio, quinoa, or oil palm.

In one embodiment, the seed weight, seed size and/or yield of the plant increases at least 0.1%, 0.5%, 1.0%, 1.5%, 2.0%, 2.5%, 3.0%, 3.5%, 4.0%, 4.5, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, or more compared to a control plant. The control plant is a plant which does not express the dominant negative KRP, biologically active variants, or fragments thereof.

In other embodiments, new plants can be derived from a cross wherein at least one parent is a transgenic plant of the present invention with increased seed weight, seed size, seed number and/or yield as described herein using breeding methods described elsewhere herein. Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety.

The present invention also provides a seed, a fruit, a plant population, a plant part, a plant cell and/or a plant tissue culture derived from the transgenic plants as described herein.

Modern plant tissue culture is performed under aseptic conditions under filtered air. Living plant materials from the environment are naturally contaminated on their surfaces (and sometimes interiors) with microorganisms, so surface sterilization of starting materials (explants) in chemical solutions (usually alcohol or bleach) is required. Explants are then usually placed on the surface of a solid culture medium, but are sometimes placed directly into a liquid medium, particularly when cell suspension cultures are desired. Solid and liquid media are generally composed of inorganic salts plus a few organic nutrients, vitamins and plant hormones. Solid media are prepared from liquid media with the addition of a gelling agent, usually purified agar.

The composition of the medium, particularly the plant hormones and the nitrogen source (nitrate versus ammonium salts or amino acids) have profound effects on the morphology of the tissues that grow from the initial explant. For example, an excess of auxin will often result in a proliferation of roots, while an excess of cytokinin may yield shoots. A balance of both auxin and cytokinin will often produce an unorganized growth of cells, or callus, but the morphology of the outgrowth will depend on the plant species as well as the medium composition. As cultures grow, pieces are typically sliced off and transferred to new media (subcultured) to allow for growth or to alter the morphology of the culture. The skill and experience of the tissue culturist are important in judging which pieces to culture and which to discard. As shoots emerge from a culture, they may be sliced off and rooted with auxin to produce plantlets which, when mature, can be transferred to potting soil for further growth in the greenhouse as normal plants.

The transgenic plants of the present invention can be used for many purposes. In one embodiment, the transgenic plant is used as a donor plant of genetic material which can be transferred to a recipient plant to produce a plant which has the transferred genetic material and has also increased seed weight, seed size, seed number and/or yield. Any suitable method known in the art can be applied to transfer genetic material from a donor plant to a recipient plant. In most cases, such genetic material is genomic material.

In one embodiment, the whole genome of the transgenic plants of the present invention is transferred into a recipient plant. This can be done by crossing the transgenic plants to a recipient plant to create a F1 plant. The F1 plant can be further selfed and selected for one, two, three, four, or more generations to give plants with increased seed weight, seed size, seed number and/or yield.

In another embodiment, at least the parts containing the transgene of the donor plant's genome are transferred. This can be done by crossing the transgenic plants to a recipient plant to create a F1 plant, followed with one or more backcrosses to one of the parent plants to plants with the desired genetic background. The progeny resulting from the backcrosses can be further selfed and selected to give plants with increased seed weight and/or seed size. In one embodiment, the recipient plant is an elite line having one or more certain agronomically important traits.

### Plant Transformation

The polynucleotides of the present invention can be transformed into a plant. The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector.

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. No. 5,204,253, U.S. Pat. No. 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed.

For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. No. 5,767,378; U.S. Pat. No. 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. No. 5,034,322, U.S. Pat. No. 6,174,724 and U.S. Pat. No. 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631 (1990), U.S. Pat. No. 4,795,855, U.S. Pat. No. 5,378,824 and U.S. Pat. No. 6,107,549). In addition, the dhfr gene, which confers resistance to the anticancer agent methotrexate, has been used for selection (Bourouis et al., EMBO J. 2(7): 1099-1104 (1983).

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit specific promoters, Ap3 promoter, heat shock promoters, seed specific promoters, etc. can also be used.

Either a gamete specific promoter, a constitutive promoter (such as the CaMV or Nos promoter), an organ specific promoter (e.g., stem specific promoter), or an inducible promoter is typically ligated to the protein or antisense encoding region using standard techniques known in the art. The expression unit may be further optimized by employing supplemental elements such as transcription terminators and/or enhancer elements. The expression cassette can comprise, for example, a seed specific promoter (e.g. the phaseolin promoter (U.S. Pat. No. 5,504,200). The term “seed specific promoter”, means that a gene expressed under the control of the promoter is predominantly expressed in plant seeds with no or no substantial expression, typically less than 10% of the overall expression level, in other plant tissues. Seed specific promoters have been well known in the art, for example, U.S. Pat. Nos. 5,623,067, 5,717,129, 6,403,371, 6,566,584, 6,642,437, 6,777,591, 7,081,565, 7,157,629, 7,192,774, 7,405,345, 7,554,006, 7,589,252, 7,595,384, 7,619,135, 7,642,346, and US Application Publication Nos. 20030005485, 20030172403, 20040088754, 20040255350, 20050125861, 20050229273, 20060191044, 20070022502, 20070118933, 20070199098, 20080313771, and 20090100551.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a preexisting vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet. 1:561-573 (1982)). The resulting expression unit is ligated into or otherwise constructed to be included in a vector that is appropriate for higher plant transformation. One or more expression units may be included in the same vector. The vector will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, are generally also included to allow the vector to be cloned in a bacterial or phage host, preferably a broad host range prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

To introduce a desired gene or set of genes by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Agrobacterium tumefaciens is a naturally occurring bacterium that is capable of inserting its DNA (genetic information) into plants, resulting in a type of injury to the plant known as crown gall. Most species of plants can now be transformed using this method, including cucurbitaceous species.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by John Sanford, Edward Wolf, and Nelson Allen. It and its registered trademark are now owned by E. I. du Pont de Nemours and Company. Most species of plants have been transformed using this method.

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

General transformation methods, and specific methods for transforming certain plant species (e.g., maize, rice, wheat, barley, soybean) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated by reference in its entirety.

### Breeding Methods

Classic breeding methods can be included in the present invention to introduce one or more recombinant KRPs of the present invention into other plant varieties, or other close-related species that are compatible to be crossed with the transgenic plant of the present invention.

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable, traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic varies widely. In practice, numbers of parental lines range from 10 to several hundred, with 100-200 being the average. Broad based synthetics formed from 100 or more clones would be expected to be more stable during seed multiplication than narrow based synthetics.

Pedigreed varieties. A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Materials and Methods

### I. Insect Cells and Media

The baculovirus expression system is a versatile eukaryotic system for heterologous gene expression. This system provides correct protein folding, disulfide bond formation and other important post-translational modifications. All methods were taken from the Baculovirus expression vector system: Procedures and methods manual. (BD Biosciences, Pharmingen, San Diego, Calif. 6th Ed.). Sf9 insect cells were grown at 27° C. in TNM-FH insect cell media (BD Biosciences) for the reported studies. It should be noted that alternative media are well known to the skilled artisan and are also useful. Similarly, alternative insect cell lines such as Sf21 and High Five™ cells will also work for virus production and protein production.

### II. Western Blots and IPs

The recombinant protein expressed in insect cells was monitored by Western blot. Protein extracts (35 μg) were boiled in the presence of Laemmli buffer, run on 10% or 12% SDS-PAGE gels and transferred to a PVDF membrane using a submerged transfer apparatus (BioRad). Following the transfer, the membrane was blocked in TBS-T (25 mM Tris pH 7.5; 75 mM NaCl; 0.05% Tween) containing 5% non-fat dry milk powder. Primary antibody was used at 1:1000 dilution overnight in TBS-T blocking buffer. Blots were washed three times 15 minutes at room temperature. An appropriate secondary antibody conjugated to horse radish peroxidase (HRP) was used at 1:10,000 dilution in TBS-T blocking buffer. Blots were incubated in secondary antibody for 1 hour and then washed three times in TBS-T, 15 min each. Blots were then processed as described in the ECL system protocol (Amersham Biosciences). Antibodies commonly used were: anti-flag M2 monoclonal antibody (Sigma), anti-HA monoclonal or polyclonal antibody (Babco), anti-PSTAIR antibody (Sigma-Aldrich), anti-myc 9E10 monoclonal or polyclonal (A-14) (Santa Cruz Biotechnology). Secondary antibodies used were anti-mouse IgG-HRP, and anti-rabbit IgG-HRP (GE Healthcare).

### III. Baculovirus Vector Construction

The Baculovirus system was Bac-to-bac (Invitrogen). Alternative Baculovirus genomes can also be used. All bacmids containing our genes of interest were independently transfected into 293 cells using lipid based transfection reagents such as Fugene or Lipofectamine. S. frugiperda Sf9 cells were seeded at 9×10⁶ cells on 60 mm dish and transiently transfected with 1 μg bacmid using 3 μl Fugene 6 transfection reagent according to the manufacturer's protocol (Roche Diagnostics). After 4 hours of transfection the Fugene/DNA solution was removed and replaced with 3 ml of TNM-FH media. Four (4) days later, the supernatant was collected and subsequently used to infect more cells for amplification of the virus. This amplification was repeated until the virus titer was at least 10⁹ virus particles/ml. The virus was amplified by infecting Sf9 cells at a multiplicity of infection (moi) of <1. The virus titer was monitored using light and fluorescence microscopy.

The Z. mays cyclins and CDKs were epitope-tagged to enable identification by Western blot and for immunoprecipitation experiments. The Z. mays cyclins and/or CDKs can also be used lacking the tags. Other compatible transfer vector systems can also be used.

Z. mays Cyclin D4 (ZmCyclinD4)

ZmCyclinD4 cDNA sequence (pTG1702) was codon optimized for expression in insect cells. At the 5′ end, an optimized Kozak sequence was added to boost protein expression. Immediately following the initiator methionine, the coding sequence for the FLAG epitope, DYKDDDDKG (SEQ ID NO: 45), was added. The 5′ end was flanked by a SpeI site and immediately following the stop codon on the 3′ end a XhoI site was introduced. The SpeI/XhoI fragment of pTG1702 was subcloned into the SpeI/XhoI site of the pFASTBAC expression cassette (Invitrogen). The expression cassette pTG1743 contains the FLAG-tagged ZmCyclinD4 under control of the Autographa californica multiple nuclear polyhedrosis virus (AcMNPV) polyhedrin (PH) promoter for high-level expression in insect cells. pTG1743 was transformed into DH10bac cells according to the manufacturer's protocol. Successful site-specific transposition into baculovirus shuttle vector (bacmid) is indicated by white colonies generating recombinant bacmid. To confirm correct recombinants, PCR was used to confirm that the ZmCyclinD4 transgene was present. Specifically, the m13R and m13F-40 primers were used in a standard PCR reaction using the Mango kit (Bioline). PCR conditions were the following: 1) 94° C. denature 4 minutes, 2) 25 cycles of 94° C. 30 seconds, 55° C. 30 seconds, 72° C. 4 minutes, 3) 10 minutes 72° C. final extension. Other transfer vector systems known to the artisan can also be used.

Z. mays Cyclin D2 (ZmCyclinD2)

ZmCyclinD2 was tagged with the FLAG epitope, DYKDDDDKG (Sigma-Aldrich) (SEQ ID NO: 45), on the N-terminus following the initiator methionine by PCR and then cloned into the pFASTBAC dual expression cassette. The expression cassette pTG932 containing the FLAG-tagged ZmCyclinD2 under control of the Autographa californica multiple nuclear polyhedrosis virus (AcMNPV) polyhedrin (PH) promoter for high-level expression in insect cells was transformed into DH10bac cells according to the manufacturer's protocol. Successful site-specific transposition into baculovirus shuttle vector (bacmid) is indicated by white colonies. To confirm correct recombinants, PCR was used to confirm that the ZmCyclinD2 transgene was present. Specifically, the m13R and m13F-40 primers were used in a standard PCR reaction using the Mango kit (Bioline). PCR conditions were the following: 1) 94° C. denature 4 minutes, 2) 25 cycles of 94° C. 30 seconds, 55° C. 30 seconds, 72° C. 4 minutes, 3) 10 minutes 72° C. final extension. Other Baculovirus transfer vector systems such as baculovirus transfer vector (BD Biosciences) can also be used for this purpose.

Z. mays Cyclin Dependent Kinase A;2 (ZmCDKA;2)

A thrombin protease cleavable tag consisting of the thrombin cleavage linker site Leu Gln Leu Val Pro Arg Gly Ser Ser Ala Gly Gly Gly (LQLVPRGSSAGGG; SEQ ID NO: 46), the hemagglutinin (HA) epitope amino acid sequence Tyr Pro Tyr Asp Val Pro Asp Tyr Ala (YPYDVPDYA; SEQ ID NO: 47) followed by a poly histidine tag Ser Ala His His His His His His His His His(SAHHHHHHHHH; SEQ ID NO: 48) was placed in the XbaI/HindIII site of pFASTBAC dual resulting in plasmid pTG860. The ZmCDKA;2 cDNA lacking a stop codon was subcloned using SpeI at the 5′ end and XbaI at the 3′ end. This places the ZmCDKA;2 cDNA coding in frame with the 3′ Thrombin/HA/His tag cassette. The expression cassette containing the tagged ZmCDKA;2 (pTG931) under control of the AcMNPV polyhedrin (PH) promoter was transformed into DH10bac cells according to the manufacturer's protocol. Successful site-specific transposition into baculovirus shuttle vector (bacmid) is indicated by white colonies. To confirm correct recombinants, PCR was used to confirm that the ZmCDKA;2 transgene was present. Specifically, the m13R and m13F-40 primers were used in a standard PCR reaction using the Mango kit (Bioline). PCR conditions were the following: 1) 94° C. denature 4 minutes, 2) 25 cycles of 94° C. 30 seconds, 55° C. 30 seconds, 72° C. 4 minutes, 3) 10 minutes 72° C. final extension. Other Baculovirus transfer vector systems such as baculovirus transfer vector (BD Biosciences) can also be used for this purpose.

Z. mays Cyclin Dependent Kinase A;1 (ZmCDKA;1)

ZmCDKA;1 cDNA sequence (pTG1701) was codon optimized for expression in insect cells. At the 5′ end, a SpeI site followed by an optimized Kozak sequence was added to boost protein expression. At the 3′ end the stop codon was omitted and a 3′ Xba I site was added. The ZmCDKA;1 cDNA (pTG1701) was subcloned into the SpeI/XbaI site of pTG860-2 giving the ZmCDKA;1 with an in-frame Thrombin-HA-HIS tag. The expression cassette containing the tagged ZmCDKA;1 (pTG1747) under control of the AcMNPV polyhedrin (PH) promoter was transformed into DH10bac cells according to the manufacturer's protocol. Successful site-specific transposition into baculovirus shuttle vector (bacmid) is indicated by white colonies. To confirm correct recombinants, PCR was used to confirm that the ZmCDKA;1 transgene was present. Specifically, the m13R and m13F-40 primers were used in a standard PCR reaction using the Mango kit (Bioline). PCR conditions were the following: 1) 94° C. denature 4 minutes, 2) 25 cycles of 94° C. 30 seconds, 55° C. 30 seconds, 72° C. 4 minutes, 3) 10 minutes 72° C. final extension. Other Baculovirus transfer vector systems such as baculovirus transfer vector (BD Biosciences) can also be used for this purpose.

### IV. Recombinant Protein Production in Insect Cells

### Production of Flag-Tagged ZmcyclinD2 or ZmCyclinD4 Protein

Flag-tagged ZmcyclinD2 or ZmCyclinD4 was achieved by infecting S. frugiperda Sf9 cells with ZmcyclinD2 or ZmCyclinD4 baculovirus. To this end, Sf9 cells grown in suspension at 2×10⁶/ml were infected with recombinant baculovirus at an MOI>5 (but other higher or slightly lower MOIs will also work) for about 2-3 days and then harvested. Cells were collected and centrifuged at 3000 rpm at 4° C. The cell pellet was washed with fresh media and then centrifuged at 3000 rpm at 4° C. The pellet was frozen at −80° C. or immediately lysed. Lysis buffer consisted of 20 mM Hepes pH 7.5, 20 mM NaCl, 1 mM EDTA, 20% glycerol, 20 mM MgCl₂ plus protease inhibitors (Complete Mini, EDTA free, Boehringer Mannheim), 1 tablet per 10 ml lysis buffer. The cell lysate was sonicated on ice 2 times for 15 seconds. Protein lysate was then centrifuged at 40,000 rpm in a Beckman TLA 100.2 rotor for 2 hours. The supernatant containing the Flag-tagged ZmcyclinD2 or ZmCyclinD4 were aliquoted and frozen at −20° C. Expression was monitored by Western blot using anti-Flag M2 monoclonal antibody (Sigma-Aldrich).

### Production of Tagged ZmCDKA;1 or ZmCDKA;2

This was achieved by infection of S. frugiperda Sf9 cells with ZmCDKA;1 or ZmCDKA;2 baculovirus and processed in the same manner as described above. Expression was monitored by Western blot using anti-HA monoclonal or polyclonal antibody (Babco). Expression can also be monitored by Western blot using anti-PSTAIR antibody (Sigma-Aldrich).

### Production of Kinase Complex of ZmcyclinD2/ZmCDKA;1 or ZmcyclinD2/ZmCDKA;2

An active kinase complex of ZmcyclinD2/ZmCDKA;1 or ZmcyclinD2/ZmCDKA;2 was prepared by co-infecting S. frugiperda Sf9 cells with ZmcyclinD2 and ZmCDKA;1 viruses or with ZmcyclinD2 and ZmCDKA;2 viruses (MOI>5 for each). The active complex was purified as described above. Protein expression was monitored by Western blot of insect cell extracts using anti-Flag M2 antibody or anti-HA antibody. The interaction of ZmcyclinD2/ZmCDKA;1 or ZmcyclinD2/ZmCDKA;2 was monitored by co-immunoprecipitation as described infra.

### Production of Kinase Complex of ZmcyclinD4/ZmCDKA;1 or ZmcyclinD4/ZmCDKA;2

An active kinase complex of ZmcyclinD4/ZmCDKA;1 or ZmcyclinD4/ZmCDKA;2 was prepared by co-infecting S. frugiperda Sf9 cells with ZmcyclinD4 and ZmCDKA;1 viruses or with ZmcyclinD4 and ZmCDKA;2 viruses (MOI>5 for each). The active complex was purified as described above. Protein expression was monitored by Western blot of insect cell extracts using anti-Flag M2 antibody or anti-HA antibody. The interaction of ZmcyclinD4/ZmCDKA;1 or ZmcyclinD4/ZmCDKA;2 was monitored by co-immunoprecipitation as described infra.

### V. Kinase Assay

An in vitro assay was developed to test the ability of various ZmKRP and BnKRP molecules to inhibit Zmcyclin/ZmCDK complexes.

Kinase activity in protein extracts from insect cells infected with individual baculovirus or a co-infection with the two baculovirus as described above was monitored with a standard kinase assay. Histone HI (HHI) was the principle substrate used but recombinant tobacco retinoblastoma protein (Nt Rb) could also be used as the substrate (see Koroleva et al., Plant Cell 16, 2346-79, 2004). Kinase assays were performed as follows: 7 μg of insect cell protein extract was added to a kinase buffer cocktail (KAB: 50 mM Tris pH 8.0, 10 mM MgCl₂, 100 μM ATP plus 0.5 μCi/ml ³²PγATP and 2 μg of HHI) to a final volume of 30 μl. The reactions were incubated at 27° C. for 30 minutes. The kinase reaction was stopped with an equal volume (30 μl) of 2× Laemmli buffer. [³²P] phosphate incorporation was monitored by autoradiography and/or Molecular Dynamics PhosphorImager following SDS-PAGE on 12% gels. Alternative buffer conditions for performing CDK kinase assays can also be used. (See, e.g., Wang and Fowke, Nature 386:451-452, 1997; Azzi et al., Eur. J. Biochem. 203:353-360, 1992; Firpo et al., Mol. Cell. Biol. 14:4889-4901, 1994.)

Protein extract from insect cells infected with ZmCyclinD2 or ZmCyclinD4 alone or ZmCDKA;1 or ZmCDKA;2 alone showed no kinase activity using HHI as the substrate. Insect cells co-infected with ZmcyclinD4 virus and either ZmCDKA;1 or ZmCDKA;2 virus contained a robust kinase activity. Interestingly, insect cells co-infected with ZmcyclinD2 virus and either ZmCDKA;1 or ZmCDKA;2 virus contained less kinase activity than ZmcyclinD4 virus and either ZmCDKA;1 or ZmCDKA;2. Active CDK-like (cdc2-like) kinases can also be purified from plant protein tissue extracts or from plant tissue culture cell extracts by using p13suc1 agarose beads (See Wang and Fowke, Nature 386:451-452, 1997; Azzi et al., Eur. J. Biochem. 203:353-360, 1992) and used in a similar assay described above and in competition experiments described in Examples 2 through 8 of WO2007016319.

### VI. Retrieval and Cloning of Zm KRPs 1, 2 and 5

ZmKRPs 1 and 2 sequences (Coehlo et al 2004, Cyclin-Dependent Kinase Inhibitors in Maize Endosperm and Their Potential Role in Endoreduplication, Plant Physiology, August 2005, Vol. 138, pp. 2323-2336, incorporated herein by reference in its entirety) and ZmKrp5 were synthesized by DNA2.0 with appropriate restriction endonuclease sites on the 5′ and 3′ end to facilitate cloning into the appropriate vectors.

### VII. Recombinant Zm KRP Protein Expression in Bacteria and Purification

All bacterial expression plasmids pET16b and pET16b-5MYC carrying inserts were transformed into BL21 RosettaBlue (DE3) (Novagen). Bacterial colonies from this fresh transformation was used to inoculate 400 ml of LB containing 100 μg/ml of ampicillin and grown at 37° C. When the culture reached an OD₆₀₀ between 0.6 and 0.8 recombinant protein expression was induced with 1 mM isopropyl-D-thiogalactopyranoside (IPTG). Cells were then grown at 30° C. for three hours. Cells were collected by centrifugation in a JLA 10.500 Beckman rotor. Bacterial cell pellet was either stored at −80° C. or lysed immediately. Bacteria were lysed in 10 ml Phosphate lysis buffer (100 mM Phosphate buffer pH 7.0, 150 mM NaCl, 1% Triton X100) containing protease inhibitors and lacking EDTA. The resuspended bacterial culture was lysed via a French press or repeated sonication. Lysed cells were centrifuged at 14,000 rpm in a Beckman JA20.1 rotor for 15 minutes at 4° C. Tagged KRP molecules were mainly insoluble. Insoluble tagged KRPs were solubilized in Urea buffer (8M Urea, 100 mM Phosphate buffer pH 7.0) manually with a pipette aid. Urea-insoluble proteins were eliminated by centrifugation at 14,000 rpm in a Beckman JA20.1 rotor for 15 minutes at 4° C. Tagged KRPs were purified in batch using BD Talon Co²⁺ metal affinity resin equilibrated in Urea buffer. Batch purification was incubated at 4° C. 3 hrs to overnight under slow rotation. Slurry was loaded on a column and resin was washed with 36 bed volumes of Urea buffer followed by 12 bed volumes of Urea buffer containing 5 mM Imidazole pH 7.0. Bound tagged KRP protein was eluted using Urea buffer containing 300 mM Imidazole pH 7.0. Fractions were monitored for tagged KRP by SDS-PAGE and/or by Bradford protein assay (BioRad). Refolding of the denatured tagged KRP1 was carried out using step-wise dilution dialysis. Fractions containing the majority of tagged KRP protein were combined and dialyzed in a 1M Urea, 100 mM Phosphate buffer pH 7.0, and 1 mM Dithiothreitol for 20 hrs at 4° C. Dialysis buffer was then changed to 0.5 M Urea, 100 mM Phosphate buffer pH 7.0, and 1 mM Dithiothreitol and continued for an additional 12 hrs. Recombinant protein was collected, quantified by Bradford assay and stored at 4° C.

### VIII. Mutagenesis of Zm KRPs

Site directed mutagenesis was performed according to the protocol for Stratagene's QuikChange site-directed mutagenesis kit.

To construct ZmKrp1#1723 DN#2 with multiple amino acid substitutions (F172A;P174A) (SEQ ID NO: 8), the sense ZmKrp1DN#2 (cattgacaagtacaacgccgatgccgcaaacgactgccactccc; SEQ ID NO: 17) and anti-sense ZmKrp1DN#2 (gggagagggcagtcgtttgcggcatcggcgttgtacttgtcaatg SEQ ID NO: 18) were used for QuikChange site-directed mutagenesis with ZmKrp1#1698 as the template. The mutagenesis product was sequenced to verify presence of desired mutations. The mutant product was then subcloned into the BamHI/XhoI site of pET16b-5MYC to ultimately yield ZmKrp1DN#2 (pTG1759).

To construct ZmKrp2#1724 DN#2 with multiple amino acid substitutions (F234A;F236A) (SEQ ID NO: 12), the sense ZmKrp2DN#2 (gcttccaagtacaacgccgacgccgtccg cggcgtgccc; SEQ ID NO: 20) and anti-sense ZmKrp2DN#2 (gggcacgccgcggacggcgtcggcgttgtacttggaagc; SEQ ID NO: 21) were used for QuikChange site-directed mutagenesis with ZmKrp2#1699 as the template. The mutagenesis product was sequenced to verify presence of desired mutations. The mutant product was then subcloned into the BamHI/XhoI site of pET16b-5MYC to ultimately yield ZmKrp2DN#2 (pTG1760).

To construct ZmKrp5#1725 DN#2 with multiple amino acid substitutions (F194A;P196A) (SEQ ID NO: 16), the sense ZmKrp5DN#2 (cagggagaagtacaacgcctctgccgtg aacgactgtcctctc; SEQ ID NO: 22) and anti-sense ZmKrp5DN#2 (gagaggacagtcgttcacggcagaggcgttgtacttctccctg; SEQ ID NO: 23) were used for QuikChange site-directed mutagenesis with ZmKrp5#1700 as the template. The mutagenesis product was sequenced to verify presence of desired mutations. The mutant product was then subcloned into the BamHI/XhoI site of pET16b-5MYC to ultimately yield ZmKrp5DN#2 (pTG1761).

To construct ZmKrp1#1772 DN#3 with multiple amino acid substitutions (Y170A;F172A;P174A) (SEQ ID NO: 9), the sense ZmKrp1DN#3 (ggatttcattgacaaggccaacgc cgatgccgcaaacgactgccc; SEQ ID NO: 24) and anti-sense ZmKrp1 DN#3 (gggcagtcgtttgcggcat cggcgttggccttgtcaatgaaatcc; SEQ ID NO: 25) were used for QuikChange site-directed mutagenesis with ZmKrp1#1723 as the template. The mutagenesis product was sequenced to verify presence of desired mutations. The mutant product was then subcloned into the BamHI/XhoI site of pET16b-5MYC to ultimately yield ZmKrp1DN#3 (pTG1854).

To construct ZmKrp2#1773 DN#3 with multiple amino acid substitutions (Y232A;F234A;F236A) (SEQ ID NO: 13), the sense ZmKrp2DN#3 (gcgctttgcttccaaggccaac gccgacgccgtccgcggcgtgcc; SEQ ID NO: 26) and anti-sense ZmKrp2DN#3 (ggcacgccgcggacggcgtcggcgttggccttggaagcaaagcgc; SEQ ID NO: 27) were used for QuikChange site-directed mutagenesis with ZmKrp2#1724 as the template. The mutagenesis product was sequenced to verify presence of desired mutations. The mutant product was then subcloned into the BamHI/XhoI site of pET16b-5MYC to ultimately yield ZmKrp2DN#3 (pTG1855).

To construct ZmKrp5#1774 DN#3 with multiple amino acid substitutions (Y192A; F194A; P196A) (SEQ ID NO: 18), the sense ZmKrp5DN#3 (gcttcagggagaaggccaacgcctctgcc gtgaacgactgtcctc; SEQ ID NO: 28) and anti-sense ZmKrp5DN#3 (gaggacagtcgttcacggcagaggcgttggccttctccctgaagc; SEQ ID NO: 29) were used for QuikChange site-directed mutagenesis with ZmKrp5#1725 as the template. The mutagenesis product was sequenced to verify presence of desired mutations. The mutant product was then subcloned into the BamHI/XhoI site of pET16b-5MYC to ultimately yield ZmKrp5DN#3 (pTG1856).

### IX. Corn Transformation

The constructs in superbinary Agrobacterium were maintained on minimal medium containing the antibiotics spectinomycin, rifampicin and tetracycline. Agrobacterium was streaked on LB medium with antibiotics and grown for 1-2 days.

Greenhouse-grown plants of Hi-II genotype were used as the donor material and ears were harvested 9-12 days after pollination. These were surface-sterilized with bleach solution and rinsed with sterile Milli-Q water. Immature zygotic embryos were aseptically excised from the F2 kernels of Hi-II genotype. The Agrobacterium from LB bacterial medium was collected and suspended in liquid infection medium and acetosyringone added to a final concentration of 100 μM. Zygotic embryos were immersed in the Agrobacterium suspension to start the bacterial infection process. Subsequently, the embryos were cultured with the scutellum side up onto the surface of co-cultivation medium and incubated in the dark for 4 days. Embryos were transferred to resting medium for 3 days followed by culturing these on selection medium containing Bialaphos. Explants were sub-cultured to fresh medium every 2 weeks and maintained in the dark at 28° C. Herbicide resistant callus was selected and cultured on regeneration media to initiate shoot regeneration. In most cases, multiple shoots from subcultured callus of a single source-embryo were carried through the regeneration process to produce replicate plants, or “clones”, of a single “event”. Although it is recognized that multiple clones derived from a single Agrobacterium-infected embryo do not always represent identical transgenic events of equal patterns for T-DNA integration into the maize genome, commonly this is the case.

The regenerated plants were transferred to 25×150 mm test tubes containing growth and rooting medium. Callus and leaves of regenerated plants were confirmed to be transformed by testing with QuickStix strips for LibertyLink. Plantlets with healthy roots were transferred into 4 inch pots containing Metro-mix 360 and maintained in the greenhouse. At 4-5 leaf stage, plants were transferred to 3 gallon pots and grown to maturity. The plants were self-pollinated and T1 seed collected ˜35 days post-pollination.

### Example 1

### Effects of BnKRP Mutants and Maize KRPs Mutants on Maize Wild-Type KRPs

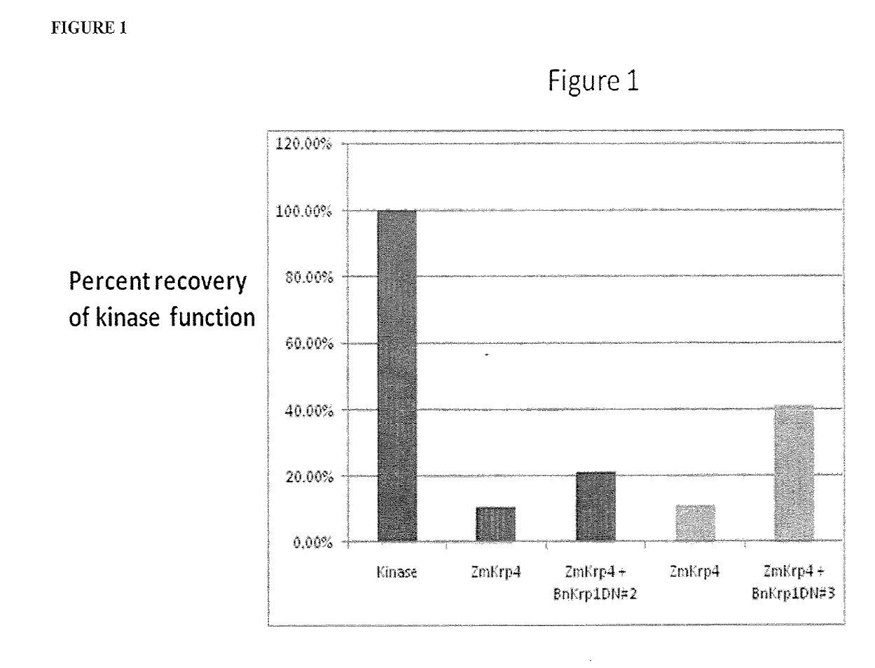

In an in vitro assays, BnKRP1 DN#2 [F151A;F153A] and BnKRP1 DN#3 [Y149A;F151A;F153A] act as dominant negative proteins against wild-type ZmKRP4 if the AtCyclin D2;1/AtCDKA complex is used (FIG. 1).

Due to the results in FIG. 1, it was hypothesized that BnKRP1 DN#2 or BnKRP1 DN#3 could function as dominant negative proteins when codon optimized for corn in corn plants. Six constructs were built to transform into corn (constructs not presented).

Field trials were conducted to see if codon optimized BnKRP1 DN#2 [F151A;F153A] or BnKRP1 DN#3 [Y149A;F151A;F153A] driven by embryo, endosperm or constitutive promoters could lead to increased seed yield. None of the constructs showed a positive effect for yield (data not provided). Without wishing to be bound by the theory, one hypothesis for the ineffectiveness of the codon optimized BnKRP1 DN#2 [F151A;F153A] or BnKRP1 DN#3 [Y149A;F151A;F153A] was that Brassica napus proteins do not act as dominant negatives against corn cyclin/CDK complexes in corn plants.

This hypothesis was tested by generating corn cyclin/CDK complexes and corn KRP DNs to test in the in vitro assay. Details of cloning these genes and production of the corn proteins are provided in the Materials and Methods, above, and elsewhere herein.

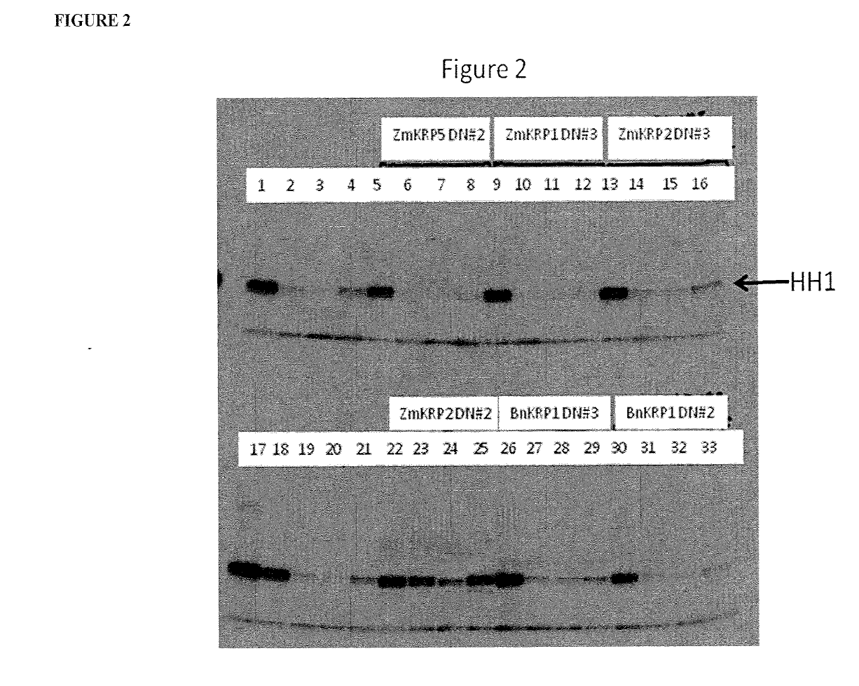

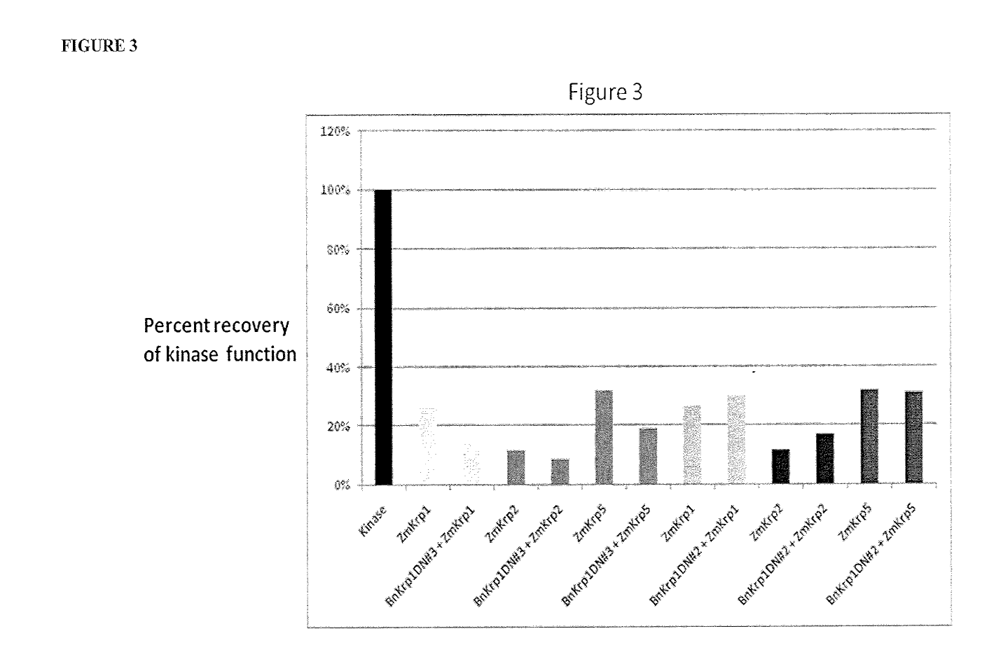

When ZmCyclinD4/ZmCDKA;1 kinase complex is used in the in vitro assay, neither codon optimized BnKRP1 DN#2 [F151A;F153A] nor BnKRP1 DN#3 [Y149A;F151A;F153A] protects the corn-specific ZmCyclinD4/ZmCDKA;1 kinase complex, while ZmKRP2 DN#2 does protect the complex (FIGS. 2-3).

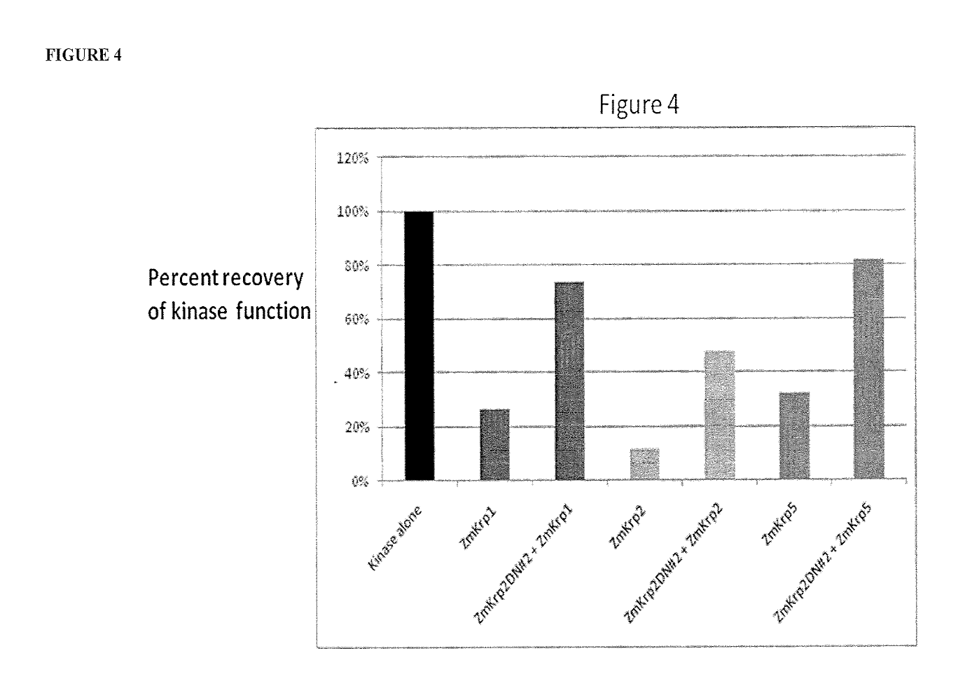

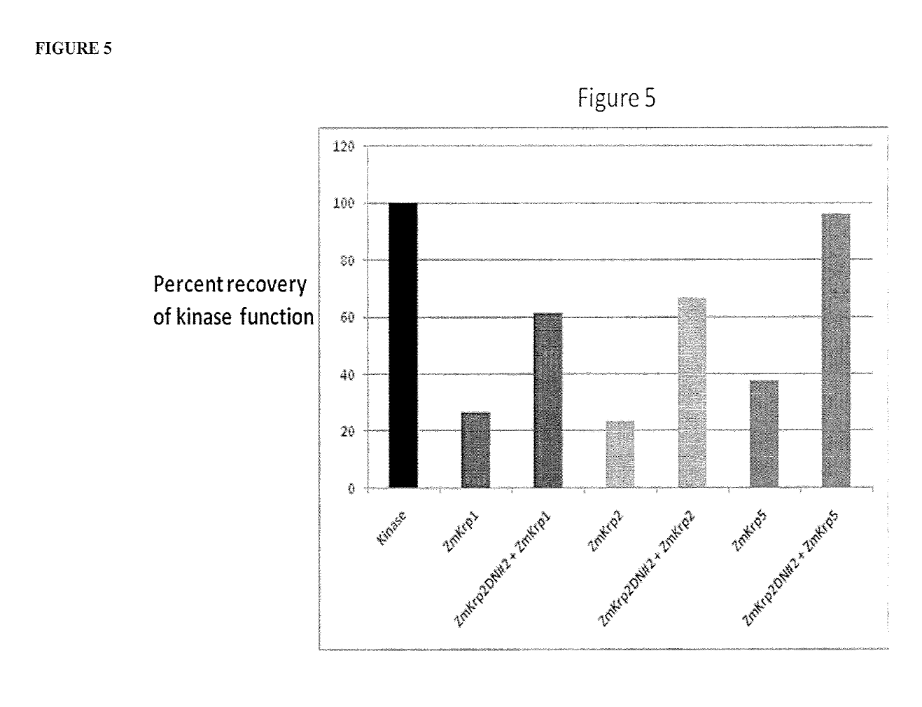

In addition, similar to codon optimized BnKRP1 DN#2 or BnKRP1 DN#3, ZmKRP1 DN#3, ZmKRP2 DN#3 and ZmKRP5 DN#2 also failed to behave as dominant negative proteins against wild-type ZmKRP1, 2 or 5 (Table 5 below and FIGS. 4-5).

TABLE 5
Biological activity of Zm KRP1, 2 and
5 DNs on maize Cyclin/CDK complexes

| Construct | | Inhibition of | Dominant |
|---|---|---|---|
| Number | Krp Mutation | Kinase Activity | Negative?¹ |
| 1732 | ZmKRP1 wild-type | +++ | N/A² |
| 1759 | ZmKrp1 DN#2 | − | − |
| 1854 | ZmKrp1 DN#3 | − | − |
| 1733 | ZmKrp2 wild-type | +++ | N/A |
| 1760 | ZmKrp2 DN#2 | − | +++ |
| 1855 | ZmKrp2 DN#3 | − | − |
| 1734 | ZmKRP5 wild-type | ++ | N/A |
| 1761 | ZmKrp5 DN#2 | − | − |
| 1856 | ZmKrp5 DN#3 | − | − |

¹Protection of ZmCyclinD4/ZmCDKA; 1 or ZmCyclinD4/ZmCDKA; 2 from inhibition by wild-type Zm KRP.
²N/A means not applicable.

### Example 2

### Expression of ZmKRP2 DN#2 in Maize

With this new in vitro data showing that ZmKRP2 DN#2 was an effective dominant negative against wild-type ZmKRPs 1, 2, and 5 in the presence of corn cyclin/CDK complexes, new constructs were built to transform into corn to evaluate for seed yield increase.

### Recombinant Expression Vectors

Table 6 shows corn constructs that were built to test the efficacy of ZmKRP2 DN#2 for seed yield increase. As controls, constructs containing ZmKRP1 DN#2 or Zm KRP2 DN#3 were also built. The promoters chosen were embryonic axis, embryo/aleurone, aleurone, endosperm, or constitutive. For ZmKRP2 DN#2, stack constructs were also built to combine expression from these various promoters. The timing and tissue specificity of each promoter is listed in Table 7.

TABLE 6
Corn constructs to test for seed yield increase
in corn inbred and hybrid field trials

| Construct | Promoter | Gene |
|---|---|---|
| TGZM101 | Zm Oleosin | ZmKRP1 DN#2 |
| TGZM103 | HvPER1 | ZmKRP1 DN#2 |
| TGZM105 | Zm Oleosin | ZmKRP2 DN#2 |
| TGZM106 | Zm Oleosin | ZmKRP2 DN#3 |
| TGZM107 | HvPER1 | ZmKRP2 DN#2 |
| TGZM108 | HvPER1 | ZmKRP2 DN#3 |
| #270 | END2 | ZmKRP2 DN#2 |
| #271 (stack) | END2 | ZmKRP2 DN#2 |
| | Zm Oleosin | ZmKRP2 DN#2 |
| #898 (stack) | ZmLEC1 | ZmKRP2 DN#2 |
| | Zm Oleosin | ZmKRP2 DN#2 |
| #272 | CZ19B1 | ZmKRP2 DN#2 |
| #951 (stack) | EEP1 | ZmKRP2 DN#2 |
| | PP1A | ZmKRP2 DN#2 |
| #952 (stack) | END2 | ZmKRP2 DN#2 |
| | Zm Oleosin | ZmKRP2 DN#2 |
| | EEP1 | ZmKRP2 DN#2 |
| | PP1A | ZmKRP2 DN#2 |

Zm = Zea mays
Hv = Hordeum vulgare

TABLE 7
Temporal and tissue expression of promoters

| Promoter | Temporal expression | | Tissue expression |
|---|---|---|---|
| Zm Oleosin | 10 | DAP-maturity | embryo/aleurone |
| HvPER1 | 20 | DPA-maturity | embryo/aleurone |
| END2 | 6-40 | DAP | aleurone |
| ZmLEC1 | 8-15 | DAP | embryonic axis |
| CZ19B1 | 13-40 | DAP | endosperm |
| PP1A | 12-40 | DAP | outer endosperm |
| EEP1 | 3-15 | DAP | endosperm |

DAP = days after pollination
DPA = days post anthesis

### Constructs

### TGZM101

Zm KRP2 DN#2-nos 3′ UTR from pTG1807 (Zm OLE pr-Zm KRP2 DN#2-nos 3′ UTR in pENTR2b, see below) was replaced with Zm KRP1 DN#2-nos 3′ UTR from pTG1763 (see below). The resulting plasmid, pTG1815 (Zm OLE pr-Zm KRP1 DN#2-nos 3′ UTR in pENTR2b) was recombined with modified pSB1 to create TGZM101 (pTG1820).

### TGZM103

Synthesized Zm KRP1 DN#2 (pTG1723) was ligated with the nos 3′ UTR from pTG1084 to create pTG1763 (Zm KRP1 DN#2-nos 3′ UTR in pCR Blunt). Zm KRP1 DN#2-nos 3′ UTR was moved from pTG1763 to pENTR2b to create pTG1777. The HvPER1 promoter from pTG1766 was ligated to Zm KRP1 DN#2-nos 3′ UTR from pTG1777 to create pTG1780 (HvPER1 pr-Zm KRP1 DN#2-nos 3′ UTR in pENTR2b). HvPER1 pr-Zm KRP1 DN#2-nos 3′ UTR from pTG1780 was recombined with modified pSB1 to create TGZM103 (pTG1789).

### TGZM105

Synthesized Zm KRP2 DN#2 (pTG1724) was ligated with the nos 3′ UTR from pTG1084 to create pTG1764 (Zm KRP2 DN#2-nos 3′ UTR in pCR Blunt). The Zm oleosin promoter (Zm OLE) was ligated with Zm KRP2 DN#2-nos 3′ UTR from pTG1764 to create pTG1802 (Zm OLE pr-Zm KRP2 DN#2-nos 3′ UTR in pCR Blunt). Zm OLE pr-Zm KRP2 DN#2-nos 3′ UTR from pTG1802 was moved to pENTR2b to create pTG1807. Zm OLE pr-Zm KRP2 DN#2-nos 3′ UTR from pTG1807 was recombined with modified pSB1 to create TGZM105 (pTG1808).

### TGZM106

Zm KRP2 DN#2-nos 3′ UTR from pTG1807 (Zm OLE pr-Zm KRP2 DN#2-nos 3′ UTR in pENTR2b, see above) was replaced with Zm KRP2 DN#3-nos 3′ UTR from pTG1796 (see below). The resulting plasmid, pTG1816 (Zm OLE pr-Zm KRP2 DN#3-nos 3′ UTR in pENTR2b) was recombined with modified pSB1 to create TGZM106 (pTG1821).

### TGZM107

Zm KRP2 DN#2-nos 3′ UTR was moved from pTG1764 to pTG1780 to create pTG1794 (HvPER1 pr-Zm KRP2 DN#2-nos 3′ UTR in pENTR2b). HvPER1 pr-Zm KRP2 DN#2-nos 3′ UTR from pTG1794 was recombined with modified pSB1 to create TGZM107 (pTG1804).

### TGZM108

Synthesized Zm KRP2 DN#3 (pTG1773) was ligated with the nos 3′ UTR from pTG1084 to create pTG1796 (Zm KRP2 DN#3-nos 3′ UTR in pCR Blunt). Zm KRP2 DN#3-nos 3′ UTR was moved from pTG1796 to pTG1780 to create pTG1803 (HvPER1 pr-Zm KRP2 DN#3-nos 3′ UTR in pENTR2b). HvPER1 pr-Zm KRP2 DN#3-nos 3′ UTR from pTG1803 was recombined with modified pSB1 to create TGZM108 (pTG1806).

### Example 3

### Evaluation of Effects of Expressing ZmKRP1 and ZmKRP2 Dominant Negative Proteins in Zea mays

### Event Characterization

T0 plantlets in the HiII background were crossed to I710 and also selfed. The F1 seed were then grown in an isolated crossing block (ICB). In the ICB, the recurrent parent (I710) and F1 seed were planted. F1 plants hemizygous or null for the transgene were typed by leaf painting. The BC1F1 cross was performed in the following manner: ears on F1s were shoot bagged to prevent uncontrolled pollination. When silks emerged, they were hand pollinated using I710 pollen. Shoot bags were maintained thereafter to prevent rogue pollination. Germ weight was taken for BC1F1 transgenic and null seeds. RNA and protein levels of ZmKRP DN were determined by Taqman real-time PCR and Western blots, respectively.

### Isolated Crossing Block (ICB) Trials

The overall objective of these trials was to assess the influence of the genes being tested on productivity of grain per plant and on the two underlying yield components, kernel number per ear and average kernel dry weight.

In most cases, seed being planted were F1 hybrid seed. They were produced by crossing one type of plant (A), which carried the transgene of interest and typically contained 50% elite corn breeding germplasm background (i.e., recurring parent), with a second type of plant (B) that was a commercial elite inbred of a counter heterotic group. In other cases, seed planted were BC1 or BC2 hybrid seed, meaning that they contained 75 or 87% elite corn breeding germplasm background (i.e., recurring parent), respectively, and had been crossed to a commercial elite inbred of a counter heterotic group. In at least one case, incorporation of the last dose of the recurring parent was accomplished through a cross to a male-sterile version of the recurring parent, so progeny plants were male-sterile. F1, BC1 or BC2 Null and Transgenic hybrid seed were identified by use of a selectable marker.

In the ICB design, rows were planted with a mix of Null and Transgenic seed, so plants of these two types occurred at random positions within each row. These were considered female rows. Female plots consisted of four female rows, each 17.5 ft. long with rows 30 inches apart. Planted adjacent to each female plot were two male rows, which were planted with the recurring parent referred to above.

Two types of ICB trials were conducted. In the first type, female rows contained hybrid plants that were male-fertile. In this case, all plants in the female rows were detasselled just before tassel emergence and pollen shed. In the second type of ICB trial, female rows contained BC1 or BC2 plants that were male-sterile. These plants were not detasselled. At all ICB trial locations, plants in female rows were pollinated by pollen released from the recurring parent planted in the male rows. Therefore, each ICB field was planted in a pattern of two male rows for every four female rows. Following plant maturity and dry down, ears were harvested from plants within each female row and segregated as originating from a Null or Transgenic plant.

Individual ears were shelled and the grain collected. In some cases, the grain of individual ears were heated in a forced-air oven for 24 hours at 103° C. and subsequently weighed to record ear grain dry weight. For other ears, the ear grain was weighed and a subsample collected for moisture determination. The subsample was weighed, heated at 103° C. for 24 hours, and reweighed to determine weight loss and moisture concentration. Percent moisture of the subsample was then used to calculate the dry weight of all grain shelled from the ear.

### General Corn Yield Trial Design

Both inbred and hybrid trials can be done. Inbred trials can be done at multiple locations in a randomized design with multiple replications per event. Individual ears are harvested to determine seed yield. Hybrid trials are at multiple locations in a split plot design with 3 replications per event per location. Plots are harvested to determine seed yield.

### Results

Ear grain dry weights were determined for Null and Transgenic events of TGZm101, 103, 105, 106, 107 and 108 from ICB trials. Events TGZm101-S028 and -S033 (ZmOleosin promoter-ZmKRP1 DN#2) showed significant seed yield increases at 2 out of 4 locations. Events TGZm103-F010 and -011 (HvPer promoter-ZmKRP1 DN#2) showed significant seed yield increases at 1 out of 3 locations. Event TGZm105-A017 (ZmOleosin promoter-ZmKRP2 DN#2), showed significant seed yield increases at 1 out of 2 locations, while event TGZm105-F009 showed positive seed yield increases at 4 out of 5 locations with two locations approaching significance. Finally, event TGZm108-S004 (HvPer promoter-ZmKRP2 DN#3) showed significant seed yield increases at 1 out of 3 locations. Events of interest will be tested further in advanced ICB trials where more plots will be used to assess yield effects. See table 8 below.

As described in Example 1, mutant ZmKrp2 DN#2 was capable of protecting two specific cyclin-CDK complexes while other DN#2 mutants such as ZmKRP1 and ZmKRP5 were incapable of significantly protecting the same two kinase complexes. The seed yield increases seen with ZmKRP1 DN#2 in corn ICB trials were therefore unexpected. However, it remains possible that ZmKRP1 and ZmKRP5 DN mutants could protect other CDK complexes activated by ZmCyclins from the D1 family, D2 family (D2;2, D2;3), D3 family (D3;1, D3;2 and D3;3) or even other members of the ZmCyclin D4 family.

TABLE 8
Seed yield results from ICB trials for TGZM101-108

| | | Loc1 | Loc2 | Loc3 | Loc4 | Loc5 | | |
|---|---|---|---|---|---|---|---|---|
| | | Ear | Ear | Ear | Ear | Ear | | |
| | | Grain | Grain | Grain | Grain | Grain | | |
| | | D. Wt. | D. Wt. | D. Wt. | D. Wt. | D. Wt. | Ave. | |
| | | % of | % of | % of | % of | % of | % of | # |
| Construct | Promoter-Gene | Null | Null | Null | Null | Null | Null | Locs |
| 101-F013 | ZmOle-ZmKRP1 DN2 | 96 | — | 107 | 103 | 102 | 102 | 4 |
| 101-F029 | ZmOle-ZmKRP1 DN2 | 99 | 105 | 90^{¥} | | 107 | 100 | 4 |
| 101-S012 | ZmOle-ZmKRP1 DN2 | | — | | — | 93 | 93 | 1 |
| 101-S020 | ZmOle-ZmKRP1 DN2 | 93 | — | | — | 95 | 94 | 2 |
| 101-S022 | ZmOle-ZmKRP1 DN2 | 101 | 101 | 99 | | 101 | 101 | 4 |
| 101-S027 | ZmOle-ZmKRP1 DN2 | 101 | 106 | 112 | | 102 | 105 | 4 |
| 101-S028 | ZmOle-ZmKRP1 DN2 | 109* | 100 | 117* | | 100 | 107 | 4 |
| 101-S033 | ZmOle-ZmKRP1 DN2 | 101 | 108* | 109 | | 107* | 106 | 4 |
| 103-F010 | HvPer-ZmKRP1 DN2 | 106 | 101 | 121* | | | 109 | 3 |
| 103-F011 | HvPer-ZmKRP1 DN2 | 105* | 104 | 102 | | | 104 | 3 |
| 103-S015-06 | HvPer-ZmKRP1 DN2 | 94 | 105 | | | 100 | 100 | 3 |
| 103-S015-02 | HvPer-ZmKRP1 DN2 | 99 | 97 | | | 103 | 100 | 3 |
| 103-S017 | HvPer-ZmKRP1 DN2 | 98 | 94 | | | 95 | 96 | 3 |
| 103-S020 | HvPer-ZmKRP1 DN2 | 93 | 94 | 126* | | 99 | 103 | 4 |
| 105-A005 | ZmOle-ZmKRP2 DN2 | 103 | 102 | | | 106 | 104 | 3 |
| 105-A007 | ZmOle-ZmKRP2 DN2 | 92^{¥} | 104 | 110* | | 99 | 101 | 4 |
| 105-A017 | ZmOle-ZmKRP2 DN2 | 112* | — | 103 | — | — | 108 | 2 |
| 105-F007 | ZmOle-ZmKRP2 DN2 | 92 | 100 | 104 | | 97 | 98 | 4 |
| 105-F009 | ZmOle-ZmKRP2 DN2 | 107 | 99 | 109 | 109 | 102 | 105 | 5 |
| 105-F050 | ZmOle-ZmKRP2 DN2 | | 112* | 103 | 104 | 93^{¥} | 103 | 4 |
| 106-S002 | ZmOle-ZmKRP2 DN3 | 101 | 96 | 100 | 109* | 102 | 102 | 5 |
| 107-S029-05 | HvPer-ZmKRP2 DN2 | 96 | — | 105 | 100 | — | 100 | 3 |
| 107-S029-01 | HvPer-ZmKRP2 DN2 | 105 | 99 | 91 | — | 105 | 100 | 4 |
| 108-A005 | HvPer-ZmKRP2 DN3 | 102 | 97 | 101 | 84^{¥} | 108* | 99 | 5 |
| 108-S004 | HvPer-ZmKRP2 DN3 | 103 | 98 | 115* | | 103 | 105 | 4 |

Blank cells = no data available due to loss of events from weather or deer predation, poor null/transgenic segregation or insufficient ears for analysis.
Dashed cells = event not planted at that location.
*Significant increase in ear grain dry weight, p ≦ 0.10.
^{¥}Significant decrease in ear grain dry weight, p ≦ 0.10.

### Example 4

Based on data of Example 3, selected ear grain samples are examined for average kernel dry weight and an estimation of kernel number per ear. The former are measured by determining the dry weight of a counted number of kernels taken from each ear. Kernel number per ear is estimated by dividing the ear grain dry weight by the average kernel dry weight. Thus, grain productivity per ear and the core kernel weight and kernel number yield components are determined.

### Example 5

For each of the constructs #270, #271, #272, #898, #951, and #952 in Table 6, multiple expressing, single copy events were generated previously. Performance of hybrid events is compared to appropriate checks with the experimental design being a randomized complete block. Events are yield tested in multi-location, multi-replication trials in North America. Data to be collected include stand count, flowering dates (selected locations), barren count (selected locations), grain yield, and grain moisture at harvest. All data are analyzed using a mixed model analysis. One or more events have increased yield compared to a control line.

### Example 6

The ability of all DN KRPs to protect the various combinations of the Cyclins plus either ZmCDKA;1 or ZmCDKA;2 are tested as described in Example 1.

DN KRPs that can protect a combination of the Cyclins and CDK are identified, and used to construct expression vectors using the methods as described in Example 2, using one or more proper promoters, for example, the promoters described herein.

The expression vectors are then transformed into corn plants, and the transgenic plants are subjected to field trial to determine if any transgenic plants have increased yield, according to the methods described in Example 3.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, patent publications, and nucleic acid and amino acid sequences cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Magyar Z, Me'sza'ros T, Miskolczi P, Dea'k M, Fehe'r A, Brown S, Kondorosi E, Athanasiadis A, Pongor S, Bilgin M, et al (1997) Cell cycle phase specificity of putative cyclin-dependent kinase variants in synchronized alfalfa cells. Plant Cell 9: 223-235
Porceddu A, Stals H, Reichheld J-P, Segers G, De Veylder L, De Pinho Barrôco R, Casteels P, Van Montagu M, Inze' D, Mironov V (2001) A plant-specific cyclin-dependent kinase is involved in the control of G2/M progression in plants. J Biol Chem 276: 36354-36360
Sorrell D A, Menges M, Healy J M S, Deveaux Y, Amano X, Su Y, Nakagami H, Shinmyo A, Doonan J H, Sekine M, et al (2001) Cell cycle regulation of cyclin-dependent kinases in tobacco cultivar Bright Yellow-2 cells. Plant Physiol 126: 1214-1223
Hemerly A, de Almeida Engler J, Bergounioux C, VanMontaguM, Engler G, Inze' D, Ferreira P (1995) Dominant negative mutants of the Cdc2 kinase uncouple cell division from iterative plant development. EMBO J 14: 3925-3936
Nowack M K, Grini P E, Jakoby M J, Lafos M, Koncz C, Schnittger A (2005) A novel positive signal from the fertilization of the egg cell sets off endosperm proliferation in angiosperm embryogenesis. Nat Genet (in press)
Boudolf V, Barrôco R, de Almeida Engler J, Verkest A, Beeckman T, Naudts M, Inze' D, De Veylder L (2004a) B1-type cyclin-dependent kinases are essential for the formation of stomatal complexes in Arabidopsis thaliana. Plant Cell 16: 945-955
Kwon T. K. et al. Identification of cdk2 binding sites on the p27Kip1 cyclin-dependent kinase inhibitor. Oncogene. 1998 Feb. 12; 16(6):755-62.
Vlach J. et al. Phosphorylation-dependent degradation of the cyclin-dependent kinase inhibitor p27. EMBO J. 1997 Sep. 1; 16(17):5334-44.
Russo et al., “Crystal Structure of the p27KiP1 Cyclin-Dependent-Kinase Inhibitor Bound to the Cyclin A-Cdk2 Complex,” Nature 382:325-331 (1996).
Jasinski S. et al., Coparative molecular and functional analyses of the tobacco cyclin-dependent kinase inhibitor NtKIS1a and its spliced variant NtKIS1b. Plant Physiolo. 2002 December; 130(4): 1871-Mendenhall M D (1998) Cyclin-dependent kinase inhibitors of Saccharomyces cerevisiae and Schizosaccharomyces pombe. Curr Top Microbiol Immunol 227: 1-24
Wang H, Fowke L C, Crosby W L (1997) A plant cyclin-dependent kinase inhibitor gene. Nature 386: 451-452
Wang H, Qi Q, Schorr P, Cutler A J, Crosby W L, Fowke L C (1998) ICK1, a cyclin-dependent protein kinase inhibitor from Arabidopsis thaliana interacts with both Cdc2a and CycD3, and its expression is induced by abscisic acid. Plant J 15: 501-510
Wang H, Zhou Y, Gilmer S, Whitwill S, Fowke L C (2000) Expression of the plant cyclin-dependent kinase inhibitor ICK1 affects cell division, plant growth and morphology. Plant J 24: 613-623
Lui H, Wang H, DeLong C, Fowke L C, Crosby W L, Fobert P R (2000) The Arabidopsis Cdc2a-interacting protein ICK2 is structurally related to ICK1 and is a potent inhibitor of cyclin-dependent kinase activity in vitro. Plant J 21: 379-385
De Veylder L, Beeckman T, Beemster G T S, Krols L, Terras F, Landrieu I, Van Der Schueren E, Maes S, Naudts M, Inze' D (2001) Functional analysis of cyclin-dependent kinase inhibitors of Arabidopsis. Plant Cell 13: 1653-1667
Jasinski S, Perennes C, Bergounioux C, Glab N (2002b) Comparative molecular and functional analyses of the tobacco cyclin-dependent kinase inhibitor NtKIS1a and its spliced variant NtKIS1b. Plant Physiol 130: 1871-1882
Jasinski S, Riou-Khamlichi C, Roche O, Perennes C, Bergounioux C, Glab N (2002a) The CDK inhibitor NtKIS1a is involved in plant development, endoreduplication and restores normal development of cyclin D3;1-overexpressing plants. J Cell Sci 115: 973-982
Coelho C M, Dande R A, Sabelli P A, Sun Y, Dilkes B P, Gordon-Kamm W J, Larkins B A (2005) Cyclin-dependent kinase inhibitors in maize endosperm and their potential role in endoreduplication. Plant Physiol 138: 2323-2336
Weinl C, Marquardt S, Kuijt S J H, Nowack M K, Jakoby M J, Hu\ddot{}lskamp M, Schnittger A (2005) Novel functions of plant cyclin-dependent kinase inhibitors, ICK1/KRP1, can act non-cell-autonomously and inhibit entry into mitosis. Plant Cell 17: 1704-1722
Ormenese S, de Almeida Engler J, De Groodt R, De Veylder L, Inze' D, Jacqmard A (2004) Analysis of the spatial expression pattern of seven Kip related proteins (KRPs) in the shoot apex of Arabidopsis thaliana. Ann Bot (Lond) 93: 575-580
Coqueret O (2003) New roles for p21 and p27 cell-cycle inhibitors: a function for each cell compartment? Trends Cell Biol 13: 65-70
Richard C, Granier C, Inze' D, De Veylder L (2001) Analysis of cell division parameters and cell cycle gene expression during the cultivation of Arabidopsis thaliana cell suspensions. J Exp Bot 52: 1625-1633
Himanen K, Boucheron E, Vanneste S, de Almeida Engler J, Inze' D, Beeckman T (2002) Auxin-mediated cell cycle activation during early lateral root initiation. Plant Cell 14: 2339-2351
Grafi G, Larkins B A (1995) Endoreduplication in maize endosperm: involvement of M phase-promoting factor inhibition and induction of S phase-related kinases. Science 269: 1262-1264

Joube's J, Phan T-H, Just D, Rothan C, Bergounioux C, Raymond P, Chevalier C (1999) Molecular and biochemical characterization of the involvement of cyclin-dependent kinase A during the early development of tomato fruit. Plant Physiol 121: 857-869
Boudolf V, Vlieghe K, Beemster G T S, Magyar Z, Torres Acosta J A, Maes S, Van Der Schueren E, Inze' D, De Veylder L (2004b) The plant-specific cyclin-dependent kinase CDKB1;1 and transcription factor E2Fa-DPa control the balance of mitotically dividing and endoreduplicating cells in Arabidopsis. Plant Cell 16: 2683-2692
X. Hu, X. Cheng, H. Jiang, S. Zhu, B. Cheng and Y. Xiang, (2010), Genome-wide analysis of cyclins in maize (Zea mays), Genet. Mol. Res. 9 (3): 1490-1503

1. An expression vector comprising a polynucleotide having a nucleic acid sequence encoding a mutant Kinase Inhibitor Protein (KIP) Related Protein (KRP) comprising an amino acid sequence having at least one modification relative to a wild-type KRP, biologically active variant, or fragment thereof, said wild-type KRP polypeptide comprising (a) a cyclin binding region conferring binding affinity for a cyclin, and
(b) a cyclin-dependent kinase (CDK) binding region conferring binding affinity for a CDK,
wherein the cyclin and the CDK can form a complex;
wherein the wild-type KRP has at least 47% identity to Zea mays KRP1 (ZmKRP1) or KRP2 (ZmKRP2);
wherein the mutant KRP polypeptide does not substantially inhibit kinase activity of the Cyclin/CDK complex;
wherein the mutant KRP polypeptide can compete with one or more wild-type Zea mays KRPs for binding to the CDK; and optionally, the polynucleotide is operably-linked to a plant promoter.

2. The expression vector of claim 1, wherein the wild-type KRP is ZmKRP2, and wherein the mutant KRP has at least two modifications relative to ZmKRP2 (SEQ ID NO: 11) at amino acid position 234 and position 236.

3. The expression vector of claim 2, wherein the two modifications are F234A and F236A relative to wild-type ZmKRP2.

4. The expression vector of claim 1, wherein the one or more wild-type Zea mays KRPs are selected from the group consisting of ZmKRP1, ZmKRP2, ZmKRP5, and combinations thereof, and wherein the CDK is selected from the group consisting of Zea mays CDK A;1 (ZmCDKA;1, SEQ ID NO: 53), Zea mays CDK A;2 (ZmCDKA;2, SEQ ID NO: 55), or combinations thereof.

5. The expression vector of claim 1, wherein the mutant KRP polypeptide is derived from ZmKRP1 (SEQ ID NO: 7), and wherein the mutant KRP has at least two modifications at the positions corresponding to amino acid position 172 and 174 of ZmKRP1 (SEQ ID NO: 7).

6. The expression vector of claim 1, wherein the wild-type KRP is encoded by a polynucleotide sequence selected from the group consisting of:
(i) a sequence encoding ZmKRP2 (SEQ ID NO: 11), biologically active variants, and fragments thereof;
(ii) a sequence encoding a polypeptide sharing at least 47% identity to the wild-type ZmKRP1 (SEQ ID NO: 7) or ZmKRP2 (SEQ ID NO: 11), biologically active variants, and fragments thereof; and
optionally, wherein the polynucleotide sequence is codon-optimized for plant expression.

7. The expression vector of claim 1, wherein the plant promoter is a constitutive promoter, an inducible promoter, or a tissue or organ specific promoter.

8. The expression vector of claim 1, wherein the plant promoter is selected from the group consisting of promoters associated with ZmOleosin gene, Hordeum vulgare PER1 (HvPER1) gene, END2 gene, ZmLEC1 gene, CZ19B1 gene, EEP1 gene, PP1A gene, ABI3 gene and Ubiquitin gene.

9. The expression vector of claim 1, wherein the vector further comprises an enhancer sequence.

10. The expression vector of claim 9, wherein the enhancer sequence is an intron-mediated enhancement (IME) element, and wherein the IME element is between the plant promoter and the polynucleotide encoding the mutant KRP.

11. The expression vector of claim 10, wherein the IME element is the first intron of maize ADH1 gene (SEQ ID NO: 44), or functional variants or fragments thereof.

12. A method for increasing average seed size, seed number, and/or yield in a plant comprising incorporating into the plant a polynucleotide sequence encoding a mutant KRP comprising an amino acid sequence having at least one modification relative to a wild-type KRP, biologically active variant, or fragment thereof, said wild-type KRP polypeptide comprising
(a) a cyclin binding region conferring binding affinity for a cyclin and
(b) a cyclin-dependent kinase (CDK) binding region conferring binding affinity for a CDK,
wherein the cyclin and the CDK can form a complex;
wherein the wild-type KRP has at least 47% identity to Zea mays KRP1 (ZmKRP1) or KRP2 (ZmKRP2);
wherein the mutant KRP polypeptide does not inhibit kinase activity of the Cyclin/CDK complex;
wherein the mutant KRP polypeptide can compete with one or more wild-type Zea mays KRPs for binding to the CDK; and optionally, the polynucleotide is operably-linked to a plant promoter.

13. The method of claim 12, wherein the plant is a monocotyledonous plant.

14. The method of claim 13, wherein the monocotyledonous plant is selected from the group consisting of corn, rice, wheat, barley, sorghum, millets, oats, ryes, triticales, buckwheats, fonio, quinoa and oil palm.

15.-17. (canceled)

18. The method of claim 12, wherein the wild-type KRP is ZmKRP2, and wherein the mutant KRP has at least two modifications relative to ZmKRP2 (SEQ ID NO: 11) at amino acid position 234 and position 236.

19.-32. (canceled)

33. A transgenic plant comprising a polynucleotide sequence encoding a mutant KRP comprising an amino acid sequence having at least one modification relative to a wild-type KRP, biologically active variant, or fragment thereof, said wild-type KRP polypeptide comprising
(a) a cyclin binding region conferring binding affinity for a cyclin and
(b) a cyclin-dependent kinase (CDK) binding region conferring binding affinity for a CDK,
wherein the cyclin and the CDK can form a complex;
wherein the wild-type KRP has at least 47% identity to Zea mays KRP1 (ZmKRP1) or KRP2 (ZmKRP2);
wherein the mutant KRP polypeptide does not inhibit kinase activity of the Cyclin/CDK complex;
wherein the mutant KRP polypeptide can compete with one or more wild-type Zea mays KRPs for binding to the CDK; and optionally, the polynucleotide is operably-linked to a plant promoter.

34. The transgenic plant of claim 33, wherein the wild-type KRP is ZmKRP2, and wherein the mutant KRP has at least two modifications relative to ZmKRP2 (SEQ ID NO: 11) at amino acid position 234 and position 236.

35. The transgenic plant of claim 34, wherein the two modifications are F234A and F236A relative to wild-type ZmKRP2.

36. The transgenic plant of claim 33, wherein the one or more wild-type Zea mays KRPs are selected from the group consisting of ZmKRP1, ZmKRP2, ZmKRP5, and combination thereof, and wherein the CDK is selected from the group consisting of Zea mays CDK A;1 (ZmCDKA;1, SEQ ID NO. 53), Zea mays CDK A;2 (ZmCDKA;2, SEQ ID NO. 55), or combination thereof.

37. The transgenic plant of claim 33, wherein the plant promoter is a constitutive promoter, a non-constitutive promoter, an inducible promoter, or a tissue or organ specific promoter.

38. The transgenic plant of claim 33, wherein the plant promoter is selected from the group consisting of promoters associated with ZmOleosin gene, Hordeum vulgare PER1 (HvPER1) gene, END2 gene, ZmLEC1 gene, CZ19B1 gene, EEP1 gene, PP1A gene, ABI3 gene and Ubiquitin gene.

39. The transgenic plant of claim 33, wherein the plant is the monocotyledonous plant selected from the group consisting of corn, rice, wheat, barley, sorghum, millets, oats, ryes, triticales, buckwheats, fonio, quinoa and oil palm.

40.-42. (canceled)

43. A seed, a fruit, a cell or a part of the transgenic plant of claim 33.

44.-50. (canceled)
